(19) United States
(12) Patent Application Publication
(10) Pub. No.: US 2025/0313637 A1
(43) Pub. Date: Oct. 9, 2025

(54) MULTISPECIFIC ANTIBODIES AND METHODS OF USE THEREOF

(71) Applicant: Dren Bio Management, Inc., Foster City, CA (US)

(72) Inventors: Xiaodi DENG, Foster City, CA (US); Meaghan HAPPER, Redwood City, CA (US); Nenad TOMASEVIC, Hillsborough, CA (US)

(21) Appl. No.: 19/086,632

(22) Filed: Mar. 21, 2025

Related U.S. Application Data
(63) Continuation of application No. PCT/US2023/075224, filed on Sep. 27, 2023.
(60) Provisional application No. 63/410,919, filed on Sep. 28, 2022.

(51) Int. Cl. C07K 16/28

(52) U.S. Cl. CPC C07K 16/2851; C07K 2317/31; C07K 2317/52; C07K 2317/522; C07K 2317/528; C07K 2317/53

(57) ABSTRACT

The present disclosure relates to multispecific antibodies, as well as polynucleotides, vectors, host cells, pharmaceutical compositions, methods of use, and methods production related thereto. In some embodiments, the multispecific antibodies use antibody CH4 domains comprising at least one amino acid substitution that promotes association between the antibody CH4 domains to favor correct heavy chain:light chain pairing.

### CROSS-REFERENCE TO RELATED APPLICATIONS

This application is a continuation application of International Application No. PCT/US2023/075224, filed on Sep. 27, 2023, which claims priority to U.S. Provisional Application No. 63/410,919, filed Sep. 28, 2022, the disclosures of each of which are incorporated herein by reference in their entirety.

### REFERENCE TO AN ELECTRONIC SEQUENCE LISTING

The contents of the electronic sequence listing (186542000901SEQLIST.xml; Size: 104,498 bytes; and Date of Creation: Mar. 18, 2025) are incorporated herein by reference in their entirety.

### FIELD

The present disclosure relates to multispecific antibodies, as well as polynucleotides, vectors, host cells, pharmaceutical compositions, methods of use, and methods production related thereto.

### BACKGROUND

Multispecific antibodies allow for the targeting of multiple factors with a single molecule, providing a multitude of opportunities for therapeutic molecules and research tools. One challenge in the design of multispecific antibodies is promoting the correct pairing between specific heavy chains and light chains that make up each individual antigen binding site that recognizes a target. A variety of approaches have been tested for promoting correct heavy chain:light chain pairing, including domain cross-over (swapping C_{H1} and C_{L} domains between heavy and light chains), replacement of the CH1 and C_{L} domains with antibody CH2 domains, replacement of the CH1 and CL domains with antibody CH3 domains, replacement of the CH1 and C_{L} domains with TCR constant domains, and engineered disulfide bonds. See, e.g., US PG Pub. Nos. 2017/0129962, 2018/0057567, 2020/0123260, and 2020/0283524; U.S. Pat. Nos. 9,527,927 and 10,982,008; and Mazor, Y. et al. (2015) MAbs 7:377-389. Other approaches have been described for promoting correct pairing between heavy chains, such as knobs-into-holes technology (introducing knob-forming and hole-forming mutations into native constant domains; see, e.g., Ridgway, J. B. B. et al. (1996) Protein Engineering 9 (7): 617-621).

Several criteria are ideally satisfied in the selection of a suitable domain to be used for exchange/cross-over of the CH1 and C_{L} regions in an engineered multispecific antibody. An optimal heterodimer domain for this purpose would ideally be stable, non-immunogenic, Ig domain-based, stoichiometric, have a high affinity for its target, and would not pair with other domains within IgG1-4.

All references cited herein, including patent applications, patent publications, and scientific literature, are herein incorporated by reference in their entirety, as if each individual reference were specifically and individually indicated to be incorporated by reference.

### BRIEF SUMMARY

The present disclosure relates to multispecific antibodies, as well as polynucleotides, vectors, host cells, pharmaceutical compositions, methods of use, and methods production related thereto. In some embodiments, the multispecific antibodies use antibody CH4 domains comprising at least one amino acid substitution that promotes association between the antibody CH4 domains to favor correct heavy chain:light chain pairing. The present disclosure describes the identification of optimal heterodimer domains that are stable, non-immunogenic, Ig domain-based, stoichiometric, have a high affinity for their target, and are not thought to not pair with other domains within IgG1-4. A search for suitable heterodimer domains identified antibody CH4 domains (e.g., IgM or IgA), which were found to have a binding interface with high structural similarity to the CH1:CK interface of a Fab fragment. Advantageously, using a CH4 domain would reduce the likelihood that the domain for promoting heterodimerization would instead pair with a domain of the antibody constant or Fc region, such as a CH1, CH2, or CH3 domain. Certain mutations can also be used to promote correct heavy chain:light chain pairing, as well as correct heavy chain:heavy chain association, leading to stable multispecific antibodies amenable to manufacturing.

In one aspect, provided herein is a multispecific antibody comprising a first arm and a second arm, wherein the first arm comprises a first antigen binding site that specifically binds a first antigen, the second arm comprises a second antigen binding site that specifically binds a second antigen, and one or both arm(s) comprise(s) a light chain:heavy chain pair in which the light chain and heavy chain both comprise an antibody constant heavy chain 4 (CH4) domain. In some embodiments, at least one of the CH4 domains comprises one or more amino acid substitution(s) that promotes association of the heavy and light chain of the arm. In some embodiments, neither of the CH4 domains is in an Fc region of the antibody. In some embodiments, the antibody CH4 domains are human antibody CH4 domains.

In one aspect, provided herein is a multispecific antibody comprising a first arm and a second arm, wherein the first arm comprises a first antigen binding site that specifically binds a first antigen, the second arm comprises a second antigen binding site that specifically binds a second antigen, and wherein at least one arm comprises: i) a first polypeptide that comprises, in an N-terminal to C-terminal direction, a structure represented by the formula:

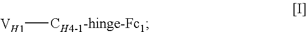

and
ii) a second polypeptide that comprises, in the N-terminal to C-terminal direction, a structure represented by the formula:

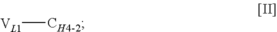

wherein V_{H1} is a first heavy chain variable (VH) domain; wherein C_{H4-1} is a first antibody constant heavy chain 4 (CH4) domain; wherein hinge is an antibody hinge region; wherein Fc₁ is a first antibody Fc region; and wherein the first VH domain and the first VL domain form the first antigen binding site. In some embodiments, at least one of the CH4 domains comprises one or more amino acid substitution(s) that promotes association of the heavy and light chain of the first arm. In some embodiments, the first and second antibody CH4 domains are human antibody CH4 domains.

In one aspect, provided herein is a multispecific antibody comprising: a) a first arm comprising: i) a first polypeptide that comprises, in an N-terminal to C-terminal direction, a structure represented by the formula:

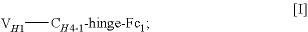

and
ii) a second polypeptide that comprises, in the N-terminal to C-terminal direction, a structure represented by the formula:

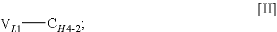

b) a second arm comprising a second antigen binding site that specifically binds a second antigen; wherein V_{H1} is a first heavy chain variable (VH) domain; wherein C_{H4-1} is a first antibody constant heavy chain 4 (CH4) domain; wherein hinge is an antibody hinge region; wherein Fc₁ is a first antibody Fc region; wherein V_{L1} is a first light chain variable (VL) domain; wherein C_{H4-2} is a second antibody CH4 domain; wherein the first VH domain and the first VL domain form a first antigen binding site that specifically binds a first antigen; and wherein the first and/or second antibody CH4 domains comprise at least one amino acid substitution that promotes association of the first and second antibody CH4 domains. In some embodiments, the second arm comprises a single domain antibody. In some embodiments, the second arm comprises a single chain antibody comprising a second VH domain and a second VL domain that make up the second antigen binding site. In some embodiments, the second arm comprises a single chain variable fragment (scFv) antibody, optionally fused with an antibody Fc region. In some embodiments, the second arm further comprises a second antibody Fc region. In some embodiments, the first and/or second antibody Fc regions comprise at least one amino acid substitution that promotes association of the first and second antibody Fc regions. In some embodiments, the first and second antibody CH4 domains are human antibody CH4 domains.

In one aspect, provided herein is a multispecific antibody comprising:
a) a first arm comprising:
   i) a first polypeptide that comprises, in an N-terminal to C-terminal direction, a structure represented by the formula:

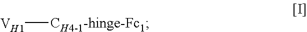

and
ii) a second polypeptide that comprises, in the N-terminal to C-terminal direction, a structure represented by the formula:

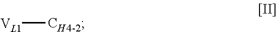

and
b) a second arm comprising:
   iii) a third polypeptide that comprises, in the N-terminal to C-terminal direction, a structure represented by the formula:

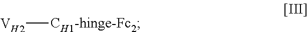

and
iv) a fourth polypeptide that comprises, in the N-terminal to C-terminal direction, a structure represented by the formula:

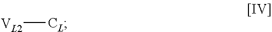

wherein:
V_{H1} is a first heavy chain variable (VH) domain;
V_{H2} is a second VH domain;
V_{L1} is a first light chain variable (VL) domain;
V_{L2} is a second VL domain;
C_{H4-1} is a first antibody constant heavy chain 4 (CH4) domain;
C_{H4-2} is a second antibody CH4 domain;
hinge is an antibody hinge region;
C_{H1} is an antibody constant heavy chain 1 (CH1) domain;
Fc₁ is a first antibody Fc region;
Fc₂ is a second antibody Fc region; and
C_{L} is an antibody light chain constant (CL) domain;
   wherein the first VH domain and the first VL domain form a first antigen binding site that specifically binds a first antigen, and the second VH domain and the second VL domain form a second antigen binding site that specifically binds a second antigen; wherein the first and/or second antibody CH4 domains comprise at least one amino acid substitution that promotes association of the first and second antibody CH4 domains; and wherein the first and/or second antibody Fc regions comprise at least one amino acid substitution that promotes association of the first and second antibody Fc regions. In some embodiments, the first and second antibody CH4 domains are human antibody CH4 domains.

In some embodiments, the first and second antigens are the same. In some embodiments, the first and second antigen binding sites specifically bind different epitopes of the same antigen. In some embodiments, the first and second antigens are different. In some embodiments, the first antigen binding site specifically binds human Dectin-1. In some embodiments, the first antigen binding site specifically binds a human Dectin-1 polypeptide that comprises the amino acid sequence of SEQ ID NO:13 or 14. In some embodiments, the first antigen binding site specifically binds human Dectin-1 expressed on the surface of a macrophage, monocyte, dendritic cell, or granulocyte. In some embodiments, the first VH domain comprises a CDR-H1 comprising the amino acid sequence GYTFTDYY (SEQ ID NO:15), a CDR-H2 comprising the amino acid sequence INPNSGDT (SEQ ID NO:16), and a CDR-H3 comprising the amino acid sequence ARNSGSYSFGY (SEQ ID NO:17). In some embodiments, the first VH domain comprises the amino acid sequence of SEQ ID NO:27. In some embodiments, the first VL domain comprises a CDR-L1 comprising the amino acid sequence QGISSW (SEQ ID NO:18), a CDR-L2 comprising the amino acid sequence GAS, and a CDR-L3 comprising the amino acid sequence QQAYSFPFT (SEQ ID NO: 20). In some embodiments, the first VL domain comprises the amino acid sequence of SEQ ID NO: 28. In some embodiments, the first VH domain comprises the amino acid sequence of SEQ ID NO: 27, and the first VL domain comprises the amino acid sequence of SEQ ID NO:28. In some embodiments, the second antigen is an antigen of a disease-causing agent. In some embodiments, the disease-causing agent is a bacterial cell, fungal cell, virus, senescent cell, tumor cell, protein aggregate, LDL particle, mast cell, eosinophil, ILC2 cell, or inflammatory immune cell. In some embodiments, the second antigen is an antigen expressed on the surface of the bacterial cell, fungal cell, senescent cell, tumor cell, mast cell, eosinophil, ILC2 cell, or inflammatory immune cell. In some embodiments, the second antigen is a surface antigen of a virus. In some embodiments, the second antigen is an antigen expressed on the surface of a cancer cell. In some embodiments, the second antigen is CD70, HER2, DLL3, NECTIN-4, TROP-2, Mesothelin, LIV-1, C-MET, FOLR1, CD20, CCR8, CD33, or EGFR. In some embodiments, the second antigen is CD20; wherein the second VH domain comprises the amino acid sequence of SEQ ID NO:29; and wherein the second VL domain comprises the amino acid sequence of SEQ ID NO:30. In some embodiments, the second antigen binding site specifically binds human Dectin-1. In some embodiments, the second antigen binding site specifically binds a human Dectin-1 polypeptide that comprises the amino acid sequence of SEQ ID NO:13 or 14. In some embodiments, the second antigen binding site specifically binds human Dectin-1 expressed on the surface of a macrophage, monocyte, dendritic cell, or granulocyte. In some embodiments, the second VH domain comprises a CDR-H1 comprising the amino acid sequence GYTFTDYY (SEQ ID NO:15), a CDR-H2 comprising the amino acid sequence INPNSGDT (SEQ ID NO:16), and a CDR-H3 comprising the amino acid sequence ARNSGSYSFGY (SEQ ID NO:17). In some embodiments, the second VH domain comprises the amino acid sequence of SEQ ID NO:27. In some embodiments, the second VL domain comprises a CDR-L1 comprising the amino acid sequence QGISSW (SEQ ID NO:18), a CDR-L2 comprising the amino acid sequence GAS, and a CDR-L3 comprising the amino acid sequence QQAYSFPFT (SEQ ID NO:20). In some embodiments, the second VL domain comprises the amino acid sequence of SEQ ID NO:28. In some embodiments, the second VH domain comprises the amino acid sequence of SEQ ID NO:27, and the second VL domain comprises the amino acid sequence of SEQ ID NO:28. In some embodiments, the first antigen is an antigen of a disease-causing agent. In some embodiments, the disease-causing agent is a bacterial cell, fungal cell, virus, senescent cell, tumor cell, protein aggregate, LDL particle, mast cell, eosinophil, ILC2 cell, or inflammatory immune cell. In some embodiments, the first antigen is an antigen expressed on the surface of the bacterial cell, fungal cell, senescent cell, tumor cell, mast cell, eosinophil, ILC2 cell, or inflammatory immune cell. In some embodiments, the first antigen is a surface antigen of a virus. In some embodiments, the first antigen is an antigen expressed on the surface of a cancer cell. In some embodiments, the first antigen is CD70, HER2, DLL3, NECTIN-4, TROP-2, Mesothelin, LIV-1, C-MET, FOLR1, CD20, CCR8, CD33, or EGFR. In some embodiments, the first antigen is CD20; wherein the first VH domain comprises the amino acid sequence of SEQ ID NO:29; and wherein the first VL domain comprises the amino acid sequence of SEQ ID NO:30. In some embodiments, the third polypeptide comprises the amino acid sequence of SEQ ID NO:34 or 35, and the fourth polypeptide comprises the amino acid sequence of SEQ ID NO:36. In some embodiments, the first polypeptide comprises the amino acid sequence of SEQ ID NO:31 or SEQ ID NO: 33, the second polypeptide comprises the amino acid sequence of SEQ ID NO:32, the third polypeptide comprises the amino acid sequence of SEQ ID NO:34 or 35, and the fourth polypeptide comprises the amino acid sequence of SEQ ID NO:36.

In some embodiments, the first polypeptide comprises, in the N-terminal to C-terminal direction, a structure represented by the formula:

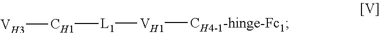

and
the multispecific antibody further comprises a fifth polypeptide that comprises, in the N-terminal to C-terminal direction, a structure represented by the formula:

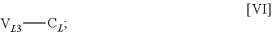

wherein:
V_{H3} is a third VH domain;
V_{L3} is a third VL domain;
C_{H1} is an antibody constant heavy chain 1 (CH1) domain;
L₁ is a linker sequence;
V_{H1} is the first VH domain;
C_{H4-1} is the first antibody CH4 domain;
hinge is the antibody hinge region;
Fc₁ is the first antibody Fc region; and
C_{L} is an antibody C_{L} domain; and
   wherein the third VH domain and the third VL domain form a third antigen binding site that specifically binds a third antigen.

In some embodiments, the second antigen and the third antigen are the same. In some embodiments, the second VH domain and the third VH domain share the same amino acid sequence; and wherein the second VL domain and the third VL domain share the same amino acid sequence. In some embodiments, the second antigen and the third antigen are different. In some embodiments, the third antigen is an antigen of a disease-causing agent. In some embodiments, the disease-causing agent is a bacterial cell, fungal cell, virus, senescent cell, tumor cell, protein aggregate, LDL particle, mast cell, eosinophil, ILC2 cell, or inflammatory immune cell. In some embodiments, the third antigen is an antigen expressed on the surface of the bacterial cell, fungal cell, senescent cell, tumor cell, mast cell, eosinophil, ILC2 cell, or inflammatory immune cell. In some embodiments, the third antigen is a surface antigen of a virus. In some embodiments, the third antigen is an antigen expressed on the surface of a cancer cell. In some embodiments, the third antigen is CD70, HER2, DLL3, NECTIN-4, TROP-2, Mesothelin, LIV-1, C-MET, FOLR1, CD20, CCR8, CD33, or EGFR. In some embodiments, the third antigen is CD20; wherein the third VH domain comprises the amino acid sequence of SEQ ID NO:29; and wherein the third VL domain comprises the amino acid sequence of SEQ ID NO:30. In some embodiments, the first polypeptide comprises the amino acid sequence of SEQ ID NO:31 or SEQ ID NO:33, and the second polypeptide comprises the amino acid sequence of SEQ ID NO:32. In some embodiments, the third antigen binding site specifically binds human Dectin-1.

In some embodiments, the multispecific antibodies of the present disclosure comprise:
a) a first arm comprising:
   i) a first polypeptide that comprises, in an N-terminal to C-terminal direction, a structure represented by the formula:

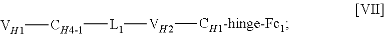

ii) a second polypeptide that comprises, in the N-terminal to C-terminal direction, a structure represented by the formula:

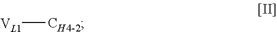

and
iii) a third polypeptide that comprises, in the N-terminal to C-terminal direction, a structure represented by the formula:

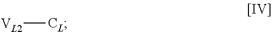

and
b) a second arm comprising:
   iv) a fourth polypeptide that comprises, in the N-terminal to C-terminal direction, a structure represented by the formula:

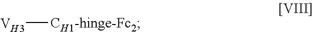

and
v) a fifth polypeptide that comprises, in the N-terminal to C-terminal direction, a structure represented by the formula:

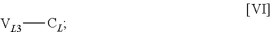

wherein:
V_{H1} is a first heavy chain variable (VH) domain;
V_{H2} is a second VH domain;
V_{H3} is a third VH domain;
V_{L1} is a first light chain variable (VL) domain;
V_{L2} is a second VL domain;
V_{L3} is a third VL domain;
L₁ is a linker sequence;
C_{H4-1} is a first antibody constant heavy chain 4 (CH4) domain;
C_{H4-2} is a second antibody CH4 domain;
C_{H1} is an antibody constant heavy chain 1 (CH1) domain;
hinge is an antibody hinge region;
Fc₁ is a first antibody Fc region;
Fc₂ is a second antibody Fc region; and
C_{L} is an antibody light chain constant (CL) domain;
   wherein the first VH domain and the first VL domain form a first antigen binding site that specifically binds a first antigen, the second VH domain and the second VL domain form a second antigen binding site that specifically binds a second antigen, and the third VH domain and the third VL domain form a third antigen binding site that specifically binds a third antigen; wherein the first and/or second antibody CH4 domains comprise at least one amino acid substitution that promotes association of the first and second antibody CH4 domains; and wherein the first and/or second antibody Fc regions comprise at least one amino acid substitution that promotes association of the first and second antibody Fc regions. In some embodiments, the first and second antibody CH4 domains are human antibody CH4 domains.

In some embodiments, the second and third antigens are different. In some embodiments, the second and third antigens are the same. In some embodiments, the second VH domain and the third VH domain share the same amino acid sequence; and wherein the second VL domain and the third VL domain share the same amino acid sequence. In some embodiments, the second and third antigen binding sites independently and specifically bind human Dectin-1. In some embodiments, the second and third antigen binding sites independently and specifically bind a human Dectin-1 polypeptide that comprises the amino acid sequence of SEQ ID NO:13 or 14. In some embodiments, the second and third antigen binding sites independently and specifically bind human Dectin-1 expressed on the surface of a macrophage, monocyte, dendritic cell, or granulocyte. In some embodiments, the second and third VH domains each comprise a CDR-H1 comprising the amino acid sequence GYTFTDYY (SEQ ID NO:15), a CDR-H2 comprising the amino acid sequence INPNSGDT (SEQ ID NO:16), and a CDR-H3 comprising the amino acid sequence ARNSGSYSFGY (SEQ ID NO:17). In some embodiments, the second and third VH domains each comprise the amino acid sequence of SEQ ID NO:27. In some embodiments, the second and third VL domains each comprise a CDR-L1 comprising the amino acid sequence QGISSW (SEQ ID NO: 18), a CDR-L2 comprising the amino acid sequence GAS, and a CDR-L3 comprising the amino acid sequence QQAYSFPFT (SEQ ID NO:20). In some embodiments, the second and third VL domains each comprise the amino acid sequence of SEQ ID NO:28. In some embodiments, the second and third VH domains each comprise the amino acid sequence of SEQ ID NO:27, and the second and third VL domains each comprise the amino acid sequence of SEQ ID NO:28. In some embodiments, the first antigen is an antigen of a disease-causing agent. In some embodiments, the disease-causing agent is a bacterial cell, fungal cell, virus, senescent cell, tumor cell, protein aggregate, LDL particle, mast cell, eosinophil, ILC2 cell, or inflammatory immune cell. In some embodiments, the first antigen is an antigen expressed on the surface of the bacterial cell, fungal cell, senescent cell, tumor cell, mast cell, eosinophil, ILC2 cell, or inflammatory immune cell. In some embodiments, the first antigen is a surface antigen of a virus. In some embodiments, the first antigen is an antigen expressed on the surface of a cancer cell. In some embodiments, the first antigen is CD70, HER2, DLL3, NECTIN-4, TROP-2, Mesothelin, LIV-1, C-MET, FOLR1, CD20, CCR8, CD33, or EGFR. In some embodiments, the first antigen is CD20; wherein the first VH domain comprises the amino acid sequence of SEQ ID NO:29; and wherein the first VL domain comprises the amino acid sequence of SEQ ID NO:30. In some embodiments, the first antigen binding site specifically binds human Dectin-1. In some embodiments, the first antigen binding site specifically binds a human Dectin-1 polypeptide that comprises the amino acid sequence of SEQ ID NO:13 or 14. In some embodiments, the first antigen binding site specifically binds human Dectin-1 expressed on the surface of a macrophage, monocyte, dendritic cell, or granulocyte. In some embodiments, the first VH domain comprises a CDR-H1 comprising the amino acid sequence GYTFTDYY (SEQ ID NO: 15), a CDR-H2 comprising the amino acid sequence INPNSGDT (SEQ ID NO:16), and a CDR-H3 comprising the amino acid sequence ARNSGSYSFGY (SEQ ID NO:17). In some embodiments, the first VH domain comprises the amino acid sequence of SEQ ID NO:27. In some embodiments, the first VL domain comprises a CDR-L1 comprising the amino acid sequence QGISSW (SEQ ID NO: 18), a CDR-L2 comprising the amino acid sequence GAS, and a CDR-L3 comprising the amino acid sequence QQAYSFPFT (SEQ ID NO:20). In some embodiments, the first VL domain comprises the amino acid sequence of SEQ ID NO:28. In some embodiments, the first VH domain comprises the amino acid sequence of SEQ ID NO:27, and the first VL domain comprises the amino acid sequence of SEQ ID NO:28. In some embodiments, the second and third antigens are antigens of a disease-causing agent. In some embodiments, the disease-causing agent is a bacterial cell, fungal cell, virus, senescent cell, tumor cell, protein aggregate, LDL particle, mast cell, eosinophil, ILC2 cell, or inflammatory immune cell. In some embodiments, the second and third antigens are antigens expressed on the surface of the bacterial cell, fungal cell, senescent cell, tumor cell, mast cell, eosinophil, ILC2 cell, or inflammatory immune cell. In some embodiments, the second and third antigen are surface antigen of a virus. In some embodiments, the second and third antigens are antigens expressed on the surface of a cancer cell. In some embodiments, the second and third antigen are each independently selected from the group consisting of CD70, HER2, DLL3, NECTIN-4, TROP-2, Mesothelin, LIV-1, C-MET, FOLR1, CD20, CCR8, CD33, and EGFR. In some embodiments, the second and third antigens are CD20; wherein the second and third VH domains each comprise the amino acid sequence of SEQ ID NO:29; and wherein the second and third VL domains each comprise the amino acid sequence of SEQ ID NO:30.

In some embodiments, the linker sequence comprises one or more glycine and/or serine residue(s). In some embodiments, the linker sequence comprises one or more repeats of the sequence GGGGS (SEQ ID NO:104).

In some embodiments, the fourth polypeptide comprises the amino acid sequence of SEQ ID NO: 40 or 41, and the fifth polypeptide comprises the amino acid sequence of SEQ ID NO:42. In some embodiments, the first polypeptide comprises the amino acid sequence of SEQ ID NO:37 or 38, the second polypeptide comprises the amino acid sequence of SEQ ID NO:39, and the third polypeptide comprises the amino acid sequence of SEQ ID NO:42. In some embodiments, the first polypeptide comprises the amino acid sequence of SEQ ID NO:43 or 44, the second polypeptide comprises the amino acid sequence of SEQ ID NO:45, and the third polypeptide comprises the amino acid sequence of SEQ ID NO:42. In some embodiments, the first polypeptide comprises the amino acid sequence of SEQ ID NO:37 or 38, the second polypeptide comprises the amino acid sequence of SEQ ID NO:39, the third polypeptide comprises the amino acid sequence of SEQ ID NO: 42, the fourth polypeptide comprises the amino acid sequence of SEQ ID NO:40 or 41, and the fifth polypeptide comprises the amino acid sequence of SEQ ID NO:42. In some embodiments, the first polypeptide comprises the amino acid sequence of SEQ ID NO:43 or 44, the second polypeptide comprises the amino acid sequence of SEQ ID NO:45, the third polypeptide comprises the amino acid sequence of SEQ ID NO:42, the fourth polypeptide comprises the amino acid sequence of SEQ ID NO: 40 or 41, and the fifth polypeptide comprises the amino acid sequence of SEQ ID NO:42.

In some embodiments, the multispecific antibodies of the present disclosure comprise:
a) a first arm comprising:
   i) a first polypeptide that comprises, in an N-terminal to C-terminal direction, a structure represented by the formula:

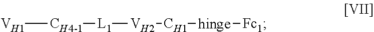

and
ii) a second polypeptide that comprises, in the N-terminal to C-terminal direction, a structure represented by the formula:

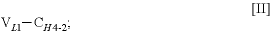

and
iii) a third polypeptide that comprises, in the N-terminal to C-terminal direction, a structure represented by the formula:

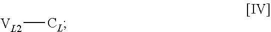

and
b) a second arm comprising:
   iv) a fourth polypeptide that comprises, in the N-terminal to C-terminal direction, a structure represented by the formula:

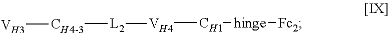

v) a fifth polypeptide that comprises, in the N-terminal to C-terminal direction, a structure represented by the formula:

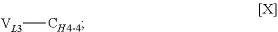

vi) a sixth polypeptide that comprises, in the N-terminal to C-terminal direction, a structure represented by the formula:

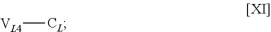

wherein:
   V_{H1} is a first heavy chain variable (VH) domain;
   V_{H2} is a second VH domain;
   V_{H3} is a third VH domain;
   V_{H4} is a fourth VH domain;
   V_{L1} is a first light chain variable (VL) domain;
   V_{L2} is a second VL domain;
   V_{L3} is a third VL domain;
   V_{L4} is a fourth VL domain;
   L₁ is a first linker sequence;
   L₂ is a second linker sequence;
   C_{H4-1} is a first antibody constant heavy chain 4 (CH4) domain;
   C_{H4-2} is a second antibody CH4 domain;
   C_{H4-3} is a third antibody CH4 domain;
   C_{H4-4} is a fourth antibody CH4 domain;
   C_{H1} is an antibody constant heavy chain 1 (CH1) domain;
   hinge is an antibody hinge region;
   Fc₁ is a first antibody Fc region;
   Fc₂ is a second antibody Fc region; and
   C_{L} is an antibody light chain constant (CL) domain;
      wherein the first VH domain and the first VL domain form a first antigen binding site that specifically binds a first antigen, the second VH domain and the second VL domain form a second antigen binding site that specifically binds a second antigen, the third VH domain and the third VL domain form a third antigen binding site that specifically binds a third antigen, and the fourth VH domain and the fourth VL domain form a fourth antigen binding site that specifically binds a fourth antigen; wherein the first and/or second antibody CH4 domains comprise at least one amino acid substitution that promotes association of the first and second antibody CH4 domains; wherein the third and/or fourth antibody CH4 domains comprise at least one amino acid substitution that promotes association of the third and fourth antibody CH4 domains; and wherein the first and/or second antibody Fc regions comprise at least one amino acid substitution that promotes association of the first and second antibody Fc regions. In some embodiments, the first, second, third, and fourth antibody CH4 domains are human antibody CH4 domains.

In some embodiments, the first and second antigens are different. In some embodiments, the third and fourth antigens are different. In some embodiments, the first and third antigens are the same. In some embodiments, the first VH domain and the third VH domain share the same amino acid sequence; and wherein the first VL domain and the third VL domain share the same amino acid sequence. In some embodiments, the second and fourth antigens are the same. In some embodiments, the second VH domain and the fourth VH domain share the same amino acid sequence; and wherein the second VL domain and the fourth VL domain share the same amino acid sequence. In some embodiments, at least one of the first, second, third, and fourth antigen binding sites specifically bind(s) human Dectin-1. In some embodiments, the second and fourth antigen binding sites independently and specifically bind human Dectin-1. In some embodiments, the first and/or third antigen(s) is/are antigen(s) of a disease-causing agent. In some embodiments, the first and/or second linker sequence(s) comprise one or more glycine and/or serine residue(s). In some embodiments, the first and second linker sequences each comprise one or more repeats of the sequence GGGGS (SEQ ID NO:104).

In some embodiments according to any of the embodiments described herein, the first and second antibody CH4 domains are human IgM CH4 domains. In some embodiments, the first and/or second antibody CH4 domains each comprise at least one amino acid substitution relative to the amino acid sequence of SEQ ID NO:1 that promotes association of the first and second antibody CH4 domains. In some embodiments, the first IgM CH4 domain comprises one or more hole-forming substitutions, and the second IgM CH4 domain comprises one or more knob-forming substitutions. In some embodiments, the first IgM CH4 domain comprises the amino acid sequence of SEQ ID NO:2, and the second IgM CH4 domain comprises the amino acid sequence of SEQ ID NO: 3. In some embodiments, the first IgM CH4 domain comprises one or more knob-forming substitutions, and the second IgM CH4 domain comprises one or more hole-forming substitutions. In some embodiments, the first IgM CH4 domain comprises the amino acid sequence of SEQ ID NO: 3, and the second IgM CH4 domain comprises the amino acid sequence of SEQ ID NO:2. In some embodiments, the first and/or second IgM CH4 domains comprise one or more engineered positively or negatively charged residues that promotes electrostatic association between the first and second IgM CH4 domains. In some embodiments, the first and second antibody CH4 domains are human IgE CH4 domains. In some embodiments, the first and/or second antibody CH4 domains each comprise at least one amino acid substitution relative to the amino acid sequence of SEQ ID NO: 4 that promotes association of the first and second antibody CH4 domains. In some embodiments, the first IgE CH4 domain comprises one or more hole-forming substitutions, and the second IgE CH4 domain comprises one or more knob-forming substitutions. In some embodiments, the first IgE CH4 domain comprises the amino acid sequence of SEQ ID NO:5, and the second IgE CH4 domain comprises the amino acid sequence of SEQ ID NO:6. In some embodiments, the first IgE CH4 domain comprises one or more knob-forming substitutions, and the second IgE CH4 domain comprises one or more hole-forming substitutions. In some embodiments, the first IgE CH4 domain comprises the amino acid sequence of SEQ ID NO:6, and the second IgE CH4 domain comprises the amino acid sequence of SEQ ID NO:5. In some embodiments, the first and/or second IgE CH4 domains comprise one or more engineered positively or negatively charged residues that promotes electrostatic association between the first and second IgE CH4 domains.

In some embodiments according to any of the embodiments described herein, the first and second antibody Fc regions are human IgG Fc regions. In some embodiments, the first and second antibody Fc regions are human IgG1, human IgG2, or human IgG4 Fc regions. In some embodiments, the first antibody Fc region comprises one or more knob-forming substitutions, and the second antibody Fc region comprises one or more hole-forming substitutions. In some embodiments, the first antibody Fc region comprises a T366W substitution, and the second antibody Fc region comprises T366S, L368A, and Y407V substitutions, numbering based on human IgG1 Fc region according to EU index. In some embodiments, the first antibody Fc region comprises one or more hole-forming substitutions, and the second antibody Fc region comprises one or more knob-forming substitutions. In some embodiments, the first antibody Fc region comprises T366S, L368A, and Y407V substitutions, and the second antibody Fc region comprises a T366W substitution, numbering based on human IgG1 Fc region according to EU index.

In another aspect, the present disclosure provides a polynucleotide encoding the multispecific antibody according to any one of the embodiments disclosed herein. In another aspect, the present disclosure provides a vector comprising the polynucleotide according to any one of the embodiments disclosed herein. In another aspect, the present disclosure provides a host cell (e.g., an isolated host cell) comprising the polynucleotide or vector according to any one of the embodiments disclosed herein. In some embodiments, the host cell is a yeast, insect, plant, or prokaryotic cell. In some embodiments, the host cell is a mammalian cell. In some embodiments, the mammalian cell is a Chinese hamster ovary (CHO) cell. In some embodiments, the host cell comprises an alpha1,6-fucosyltransferase (Fut8) or alpha-1,3-mannosyl-glycoprotein 2-beta-N-acetylglucosaminyltranferase (MGAT1) knockout. In some embodiments, the host cell overexpresses β1,4-N-acetylglucosaminyltransferase III (GnT-III). In some embodiments, the host cell further overexpresses Golgi μ-mannosidase II (ManII). In another aspect, the present disclosure provides a method of producing a multispecific antibody, comprising culturing the host cell according to any one of the embodiments disclosed herein under conditions suitable for production of the multispecific antibody. In some embodiments, the method further comprises recovering the multispecific antibody. In some embodiments, recovering the multispecific antibody comprises contacting the multispecific antibody with Protein A and eluting the multispecific antibody from the Protein A using a buffer comprising 3M MgCl₂. In some embodiments, prior to production of the multispecific antibody, the host cell is treated with kifunensine. In another aspect, the present disclosure provides a multispecific antibody produced by the method according to any one of the embodiments disclosed herein.

In another aspect, the present disclosure provides a pharmaceutical composition comprising the multispecific antibody according to any one of the embodiments disclosed herein and a pharmaceutically acceptable carrier.

In another aspect, the present disclosure provides a method of treating a disease or disorder, comprising administering an effective amount of the multispecific antibody or composition according to any one of the embodiments disclosed herein to an individual in need thereof. In another aspect, the present disclosure provides the multispecific antibody or composition according to any one of the embodiments disclosed herein for use in a method of treating a disease or disorder, comprising administering an effective amount of the multispecific antibody or composition to an individual in need thereof. In another aspect, the present disclosure provides the use of a multispecific antibody or composition according to any one of the embodiments disclosed herein in the manufacture of a medicament for treating a disease or disorder in an individual in need thereof.

In some embodiments, the multispecific antibody comprises at least one antigen binding site that specifically binds an antigen expressed on a cell surface of an immune cell and at least one antigen binding site that specifically binds a disease-causing agent. In some embodiments, the multispecific antibody comprises at least one antigen binding site that specifically binds an antigen expressed on a cell surface of a myeloid cell and at least one antigen binding site that specifically binds a disease-causing agent. In some embodiments, the multispecific antibody comprises at least one antigen binding site that specifically binds human Dectin-1 and at least one antigen binding site that specifically binds a disease-causing agent. In some embodiments, the disease-causing agent is a bacterial cell, fungal cell, virus, senescent cell, tumor cell, protein aggregate, LDL particle, mast cell, eosinophil, ILC2 cell, or inflammatory immune cell. In some embodiments, the multispecific antibody comprises at least one antigen binding site that specifically binds an antigen expressed on the surface of the bacterial cell, fungal cell, senescent cell, tumor cell, mast cell, eosinophil, ILC2 cell, or inflammatory immune cell. In some embodiments, the multispecific antibody comprises at least one antigen binding site that specifically binds a surface antigen of a virus. In some embodiments, the disease or disorder is cancer, a bacterial infection, a fungal infection, a viral infection, a mast cell disease or disorder, systemic mastocytosis, amyloidosis, or an aging-related disease or disorder. In some embodiments, the multispecific antibody comprises at least one antigen binding site that specifically binds an antigen expressed on the surface of a cancer cell. In some embodiments, the antigen expressed on the surface of a cancer cell is CD70, HER2, DLL3, NECTIN-4, TROP-2, Mesothelin, LIV-1, C-MET, FOLR1, CD20, CCR8, CD33, or EGFR. In some embodiments, the multispecific antibody comprises at least one antigen binding site that specifically binds human Dectin-1 and at least one antigen binding site that specifically binds CD20, e.g., human CD20. In some embodiments, the individual is a human.

It is to be understood that one, some, or all of the properties of the various embodiments described herein may be combined to form other embodiments of the present disclosure. These and other aspects of the present disclosure will become apparent to one of skill in the art. These and other embodiments of the present disclosure are further described by the detailed description that follows.

### BRIEF DESCRIPTION OF THE DRAWINGS

FIGS. 1A & 1B depict possible designs to drive bispecific antibody formation. FIG. 1A shows a bispecific antibody design with a conventional wild-type (WT; right) arm (e.g., targeting an immune cell antigen) paired with another arm (e.g., targeting a disease antigen) containing a designed heterodimer domain to facilitate correct chain association (left). Dashed lines indicate disulfide bonds. FIG. 1B shows existing strategies that have been used to promote correct heavy chain and light chain association in the generation of bispecific antibodies. One possibility (top), although with limited sequence space, is to use knobs-into-holes technology, which can be combined with engineered disulfide bonds on one or both fab arms to promote proper light chain:heavy chain pairing (see, e.g., US PG Pub. No. 20200123260 or Mazor, Y. et al. (2015) MAbs 7:377-389). Another possibility (bottom) is to use domain cross-over to exchange the CH1 and C_{L} domains between chains or substitute the domains with different constant regions (such as the Cross Mab design from Roche or the domain-swapped TCR from WuXi).

FIGS. 2A-2C depict the design of a bispecific antibody with pairing driven by domain swapping, enhanced by knobs-into-holes technology. FIG. 2A shows a bispecific antibody design generated by domain swapping of the CH1 and C_{L} regions with another dimerizing domain, and subsequent mutation of that homodimer to create a heterodimer interface (i.e., via knobs-into-holes technology or electrostatic steering). FIG. 2B shows a bispecific antibody design and relevant criteria for suitable domain selection (i.e., for the heterodimer domain to be used for exchange/cross-over of the CH1 and C_{L} regions). The antibody comprises a conventional wild-type (WT) arm (e.g., targeting an immune cell antigen, such as Dectin-1), paired with another arm (e.g., targeting a disease antigen) containing a designed heterodimer domain to facilitate correct chain association. Knobs-into-holes technology is used to enhance pairing of the two distinct heavy chains (i.e., between the WT and disease-targeting half-antibodies), and both domain cross-over and knobs-into-holes technology are used to enhance pairing of the heavy and light chains (i.e., between the constant domains of the disease-targeting arm). FIG. 2C compares the structures of the constant CH1 domain: CK domain interface of a Fab fragment (left) with the dimerization of constant CH4 domains of IgM (center) and shows the superimposition of both interfaces, highlighting structural similarity (right).

FIG. 3 depicts the design of a bispecific antibody (M-Fab, 2M24xCD20), with an anti-CD20 rituximab (RTX) arm and an anti-Dectin-1 2M24 arm. Knobs-into-holes technology (K and H, respectively) is used to enhance pairing of the two distinct heavy chains between the half-antibody arms, and both domain cross-over and knobs-into-holes technology are used to enhance pairing of the heavy and light chains in the 2M24 arm (MK on the light chain and MH on the heavy chain).

FIG. 4 shows purification of the M-Fab 2M24xCD20 antibody by size exclusion chromatography (SEC). 2M24xCD20 antibody was purified as a homogenous molecule with 93% monomer after elution (top). Purified 2M24xCD20 antibody was stable over 48 h at 4° C. (bottom).

FIG. 5 shows purification of the 2M24xCD20 antibody by SEC and modified elution with 3M MgCl₂ to minimize aggregation. Purified antibody was analyzed by SDS-PAGE under non-reducing (NR) or reducing (R) conditions.

FIG. 6 shows biochemical characterization of the 2M24xCD20 bispecific antibody with mass spectrometry to show the presence of the engineered heavy and light chains.

FIGS. 7A-7B show binding of bispecific 2M24xCD20 antibodies to cells expressing Dectin-1 or CD20, assessed using flow cytometry. Two different formats were used to drive heavy:light chain pairing and bispecific antibody assembly: the DuetMab format with engineered disulfide bonds (see Mazor, Y. et al. (2015) MAbs 7:377-389) (“2M24xCD20 hG1 Duet”) and the M-Fab format described herein (see, e.g., FIG. 3) (“2M24xCD20 hG1M”), as compared to monospecific anti-CD20 with hG1 Fc. Both bispecific formats used the same variable domain sequences from 2M24 (anti-Dectin-1) and rituximab (anti-CD20) antibodies. FIG. 7A shows binding of the indicated bispecific antibodies to Dectin-1-expressing HEK293 cells, quantified by mean fluorescent intensity (MFI). FIG. 7B shows binding of the indicated bispecific antibodies and control anti-CD20 monospecific antibody to CD20-expressing Raji cells, quantified by MFI. hG1:human IgG1.

FIGS. 8A-8B show two replicates of SEAP reporter assays monitoring Dectin-1 signaling induction by bispecific antibodies targeting Dectin-1 and CD20 (2M24xCD20). SEAP secretion was quantified by reading OD630. 2M24xCD20 hG1 Duet and 2M24xCD20 hG1M bispecific formats were compared alongside control bispecific targeting Dectin-1 and RSV (2M24xRSV).

FIGS. 9A-9D show alternative bispecific antibody designs with modified targets, all using knobs-into-holes technology to promote correct chain association. FIG. 9A depicts a standard 1:1 bispecific antibody design, with each half-antibody arm targeted to a single target (therefore a 1:1 ratio of antigen binding sites targeting each target). FIG. 9B depicts a modified 1:2 bispecific antibody design, with one conventional wild-type arm (e.g., targeting an immune cell target) paired to another arm that targets an immune cell target and a disease target, thereby achieving a 1:2 ratio of antigen binding sites targeting immune effector:antigen binding sites targeting disease targets. FIG. 9C depicts a modified 2:1 bispecific antibody design, with one conventional wild-type arm (e.g., targeting a disease target) paired to another arm that targets a disease target and an immune cell target, thereby achieving a 2:1 ratio of antigen binding sites targeting immune effector:antigen binding sites targeting disease targets. FIG. 9D depicts a modified 2:2 bispecific antibody design, with two arms that each target a disease target and an immune cell target, thereby achieving a 2:2 ratio of antigen binding sites targeting immune effector:antigen binding sites targeting disease targets.

FIGS. 10A-10B show an alignment of IgE CH4, IgM CH4, IgA CH3, and IgG1 CH3 domain sequences indicating the positions corresponding to hole-forming mutations T366S, L368A, and Y407V (FIG. 10A) or knob-forming mutation T366W (FIG. 10B). Shown from top to bottom are SEQ ID Nos: 4, 1, 7, and 10.

FIGS. 11A-11C show the design of a 2:1 M-Fab anti-Dectin-1/anti-Trop2 bispecific antibody (FIG. 11A) and its binding to cells expressing Trop-2 (FIG. 11B) or Dectin-1 (FIG. 11C).

FIGS. 12A-12C show activation of Dectin-1 signaling by 2:1 M-Fab anti-Dectin-1/anti-Trop2 bispecific antibody using SEAP reporter assay in the presence of cells expressing varying levels of Trop-2, including CHO cells expressing Trop2 at ˜2 million copies/cell (FIG. 12A), H2170 cells expressing hTrop2 at ˜136,000 copies/cell (FIG. 12B), and HeLa cells expressing hTrop2 at ˜25,500 copies/cell (FIG. 12C).

FIG. 13 shows lack of Dectin-1 agonism (as measured by level of TNF-alpha secreted into supernatant) by 2:1 M-Fab anti-Dectin-1/anti-Trop2 bispecific or traditional anti-Dectin-1/anti-Trop2 bispecific antibody in PBMCs in the absence of Trop2-expressing target cells after overnight incubation, as compared to 2M24 parental and other controls.

### DETAILED DESCRIPTION

Several aspects are described below with reference to example applications for illustration. It should be understood that numerous specific details, relationships, and methods are set forth to provide a full understanding of the features described herein. One having ordinary skill in the relevant art, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “comprising” as used herein is synonymous with “including” or “containing”, and is inclusive or open-ended.

Any reference to “or” herein is intended to encompass “and/or” unless otherwise stated. As used herein, the term “about” with reference to a number refers to that number plus or minus 10% of that number. The term “about” with reference to a range refers to that range minus 10% of its lowest value and plus 10% of its greatest value.

### I. Multispecific Antibodies

Certain aspects of the present disclosure relate to multispecific antibodies that comprise a first arm that comprises a first antigen binding site that specifically binds a first antigen, and a second arm that comprises a second antigen binding site that specifically binds a second antigen. In some embodiments, one or both arm(s) comprise(s) a light chain:heavy chain pair in which the light chain and heavy chain both comprise an antibody constant heavy chain 4 (CH4) domain; wherein at least one of the CH4 domains comprises one or more amino acid substitution(s) that promotes association of the heavy and light chain of the arm. In some embodiments, neither of the CH4 domains is in an Fc region of the antibody. The present disclosure is based at least in part on the discovery that antibody CH4 domains can be used to drive heterodimerization between heavy and light chains, e.g., of an arm of a multispecific antibody. In some embodiments, the CH4 domains replace (e.g., occupy the corresponding position of, between the VH domain and hinge region on the heavy chain and C-terminal to the VL domain on the light chain) heavy chain C_{H1} and/or light chain constant domains.

In some embodiments, the multispecific antibodies of the present disclosure comprise a first arm and a second arm, wherein the first arm comprises a first antigen binding site that specifically binds a first antigen, the second arm comprises a second antigen binding site that specifically binds a second antigen, and wherein at least one arm comprises: i) a first polypeptide that comprises, in an N-terminal to C-terminal direction, a structure represented by the formula:

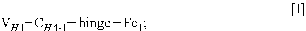

and
ii) a second polypeptide that comprises, in the N-terminal to C-terminal direction, a structure represented by the formula:

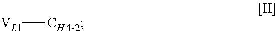

wherein V_{H1} is a first heavy chain variable (VH) domain; wherein C_{H4-1} is a first antibody constant heavy chain 4 (CH4) domain; wherein hinge is an antibody hinge region; wherein Fc₁ is a first antibody Fc region; and wherein the first VH domain and the first VL domain form the first antigen binding site. In some embodiments, at least one of the CH4 domains comprises one or more amino acid substitution(s) that promotes association of the heavy and light chain of the first arm.

In some embodiments, the multispecific antibodies of the present disclosure comprise:
a) a first arm comprising:
   i) a first polypeptide that comprises, in an N-terminal to C-terminal direction, a structure represented by the formula:

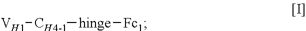

and
ii) a second polypeptide that comprises, in the N-terminal to C-terminal direction, a structure represented by the formula:

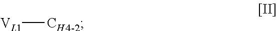

and
b) a second arm comprising a second antigen binding site that specifically binds a second antigen;
wherein V_{H1} is a first heavy chain variable (VH) domain;
wherein C_{H4-1} is a first antibody constant heavy chain 4 (CH4) domain;
wherein hinge is an antibody hinge region;
wherein Fc₁ is a first antibody Fc region;
wherein V_{L1} is a first light chain variable (VL) domain;
wherein C_{H4-2} is a second antibody CH4 domain;
wherein the first VH domain and the first VL domain form a first antigen binding site that specifically binds a first antigen; and
wherein the first and/or second antibody CH4 domains comprise at least one amino acid substitution that promotes association of the first and second antibody CH4 domains.

In some embodiments, the second arm comprises a single domain antibody. In some embodiments, the second arm comprises a single chain antibody comprising a second VH domain and a second VL domain that make up the second antigen binding site. In some embodiments, the second arm comprises a single chain variable fragment (scFv) antibody, optionally fused with a second antibody Fc region. In some embodiments, the second arm further comprises a second antibody Fc region. In some embodiments, the first and/or second antibody Fc regions comprise at least one amino acid substitution that promotes association of the first and second antibody Fc regions.

In some embodiments, the multispecific antibodies of the present disclosure comprise:
a) a first arm comprising:
   i) a first polypeptide that comprises, in an N-terminal to C-terminal direction, a structure represented by the formula;

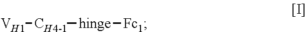

and
ii) a second polypeptide that comprises, in the N-terminal to C-terminal direction, a structure represented by the formula:

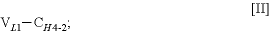

and
b) a second arm comprising:
   iii) a third polypeptide that comprises, in the N-terminal to C-terminal direction, a structure represented by the formula:

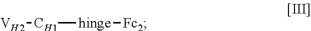

and
iv) a fourth polypeptide that comprises, in the N-terminal to C-terminal direction, a structure represented by the formula:

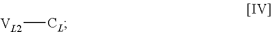

wherein:
V_{H1} is a first heavy chain variable (VH) domain;
V_{H2} is a second VH domain;
V_{L1} is a first light chain variable (VL) domain;
V_{L2} is a second VL domain;
C_{H4-1} is a first antibody constant heavy chain 4 (CH4) domain;
C_{H4-2} is a second antibody CH4 domain;
hinge is an antibody hinge region;
C_{H1} is an antibody constant heavy chain 1 (CH1) domain;
Fc₁ is a first antibody Fc region;
Fc₂ is a second antibody Fc region; and
C_{L} is an antibody light chain constant (CL) domain;
   wherein the first VH domain and the first VL domain form a first antigen binding site that specifically binds a first antigen, and the second VH domain and the second VL domain form a second antigen binding site that specifically binds a second antigen; wherein the first and/or second antibody CH4 domains comprise at least one amino acid substitution that promotes association of the first and second antibody CH4 domains.

In some embodiments, one or both CH4 domains of a pair of CH4 domains comprise at least one amino acid substitution that promotes association of the antibody CH4 domains. In some embodiments, one or both Fc regions of a pair of Fc regions comprise at least one amino acid substitution that promotes association of the Fc regions. As used herein, amino acid substitution(s) that promote association of two antibody CH4 domains or two Fc regions are those that favor heterodimerization of a cognate pair of CH4 domains/Fc regions over homodimerization. For example, one domain or region with knob-forming substitution(s) can be favored to heterodimerize with a different domain or region with hole-forming substitution(s) over homodimerizing of two knob- or hole-forming domains/regions. As such, substitution(s) that promote association favor correct heterodimerized pairing over homodimerization, such as by increasing affinity of heterodimerization and/or decreasing affinity of homodimerization.

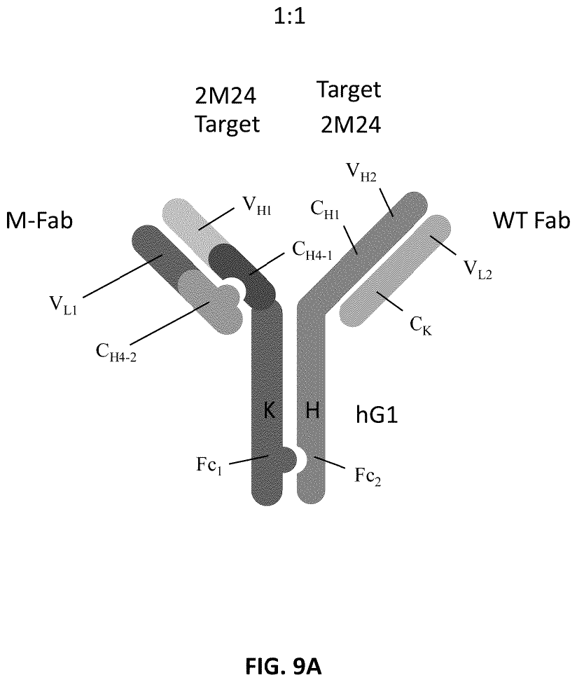

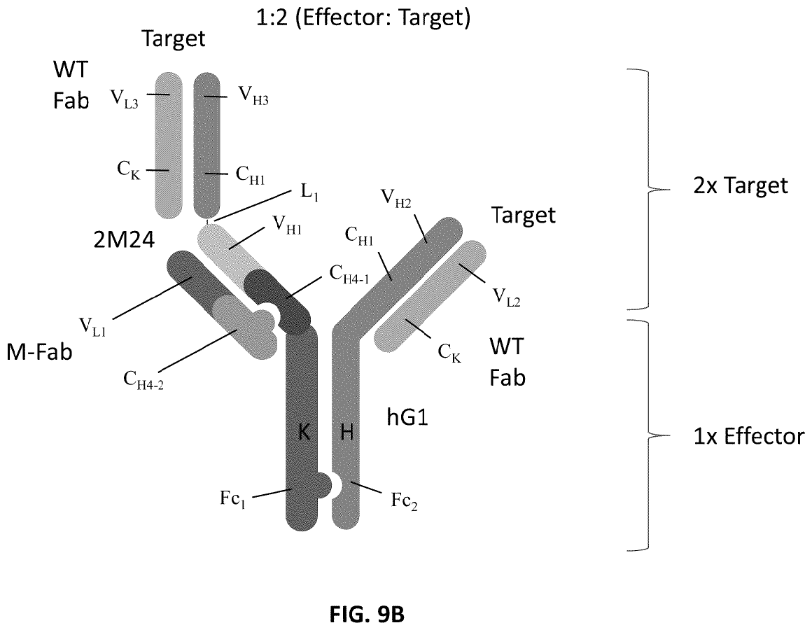

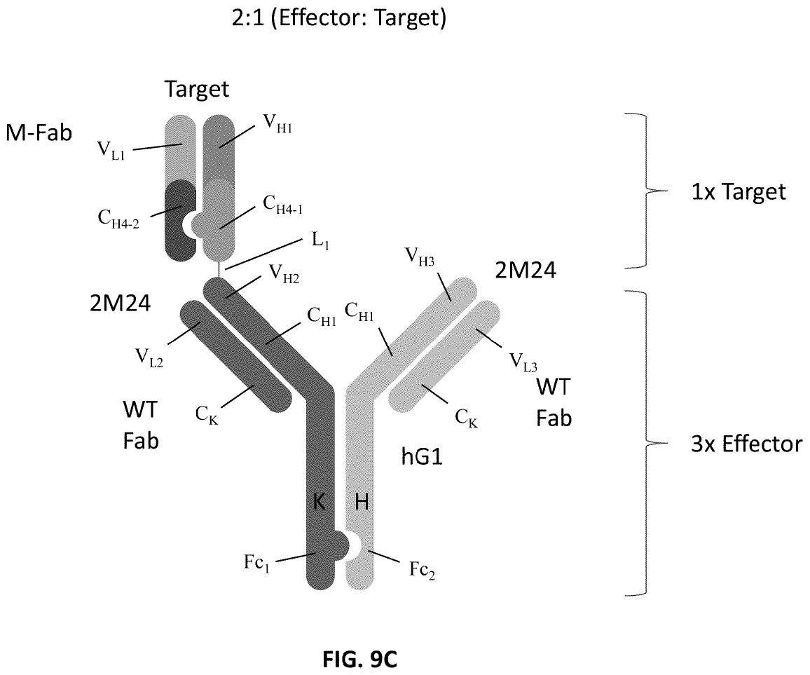

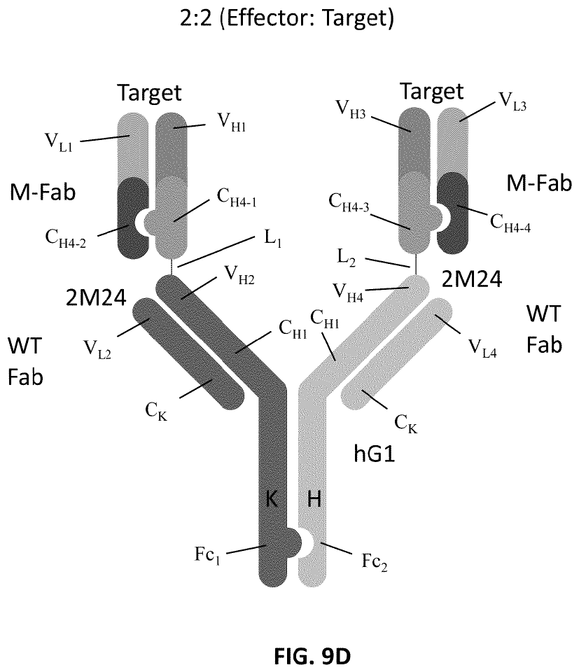

In some embodiments, the first and/or second antibody Fc regions comprise at least one amino acid substitution that promotes association of the first and second antibody Fc regions. An exemplary configuration of a multispecific binding protein is shown in FIG. 9A.

In some embodiments, one or both arm(s) of a multispecific antibody of the present disclosure can comprise an additional binding site provided by an antibody fragment fused to the multispecific antibody, e.g., at the N-terminus of one or both heavy chains. Exemplary configurations of multispecific binding proteins are shown in FIGS. 9B-9D.

In some embodiments, the first polypeptide comprises, in the N-terminal to C-terminal direction, a structure represented by the formula:

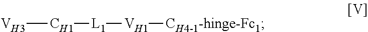

and
the multispecific antibody further comprises a fifth polypeptide that comprises, in the N-terminal to C-terminal direction, a structure represented by the formula:

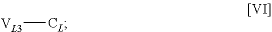

wherein:
V_{H3} is a third VH domain;
V_{L3} is a third VL domain;
C_{H1} is an antibody constant heavy chain 1 (CH1) domain;
L₁ is a linker sequence;
V_{H1} is the first VH domain;
C_{H4-1} is the first antibody CH4 domain;
hinge is the antibody hinge region;
Fc₁ is the first antibody Fc region; and
C_{L} is an antibody C_{L} domain; and
   wherein the third VH domain and the third VL domain form a third antigen binding site that specifically binds a third antigen.

An exemplary configuration of a multispecific binding protein is shown in FIG. 9B. In some embodiments, the second and third, first and second, or first and third antigens are the same. In some embodiments, the second and third, first and second, or first and third antigen binding sites are the same, e.g., comprising the same VH and VL domain sequences. In some embodiments, the first, second, and third antigens are different. In some embodiments, the first, second, and third antigen binding sites are different.

In some embodiments, the multispecific antibodies of the present disclosure comprise:
a) a first arm comprising:
   i) a first polypeptide that comprises, in an N-terminal to C-terminal direction, a structure represented by the formula:

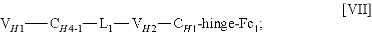

ii) a second polypeptide that comprises, in the N-terminal to C-terminal direction, a structure represented by the formula:

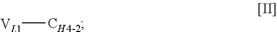

and
iii) a third polypeptide that comprises, in the N-terminal to C-terminal direction, a structure represented by the formula:

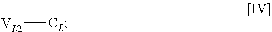

and
b) a second arm comprising:
   iv) a fourth polypeptide that comprises, in the N-terminal to C-terminal direction, a structure represented by the formula:

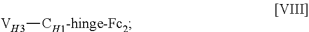

and
v) a fifth polypeptide that comprises, in the N-terminal to C-terminal direction, a structure represented by the formula:

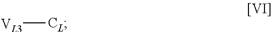

wherein:
V_{H1} is a first heavy chain variable (VH) domain;
V_{H2} is a second VH domain;
V_{H3} is a third VH domain;
V_{L1} is a first light chain variable (VL) domain;
V_{L2} is a second VL domain;
V_{L3} is a third VL domain;
L₁ is a linker sequence;
C_{H4-1} is a first antibody constant heavy chain 4 (CH4) domain;
C_{H4-2} is a second antibody CH4 domain;
C_{H1} is an antibody constant heavy chain 1 (CH1) domain;
hinge is an antibody hinge region;
Fc₁ is a first antibody Fc region;
Fc₂ is a second antibody Fc region; and
C_{L} is an antibody light chain constant (CL) domain;
   wherein the first VH domain and the first VL domain form a first antigen binding site that specifically binds a first antigen, the second VH domain and the second VL domain form a second antigen binding site that specifically binds a second antigen, and the third VH domain and the third VL domain form a third antigen binding site that specifically binds a third antigen.

In some embodiments, the first and/or second antibody CH4 domains comprise at least one amino acid substitution that promotes association of the first and second antibody CH4 domains. In some embodiments, the first and/or second antibody Fc regions comprise at least one amino acid substitution that promotes association of the first and second antibody Fc regions.

An exemplary configuration of a multispecific binding protein is shown in FIG. 9C. In some embodiments, the second and third, first and second, or first and third antigens are the same. In some embodiments, the second and third, first and second, or first and third antigen binding sites are the same, e.g., comprising the same VH and VL domain sequences. In some embodiments, the first, second, and third antigens are different. In some embodiments, the first, second, and third antigen binding sites are different.

In some embodiments, the fourth polypeptide comprises the amino acid sequence of QVQLVQSGAEVKKPGASVKVSCKASGYTFTDYYMHWVRQAPGQGLEWMGWINPNSGDT NYAQKFQGRVTMTRDTSISTAYMELSRLRSDDTAVYYCARNTGAYSFGYWGQGTLVTVSS ASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFL FPPKPKDTLMISRTPEVTCVVVDVSHEDPEVKFNWYVDGVEVHNAKTKPREEQYNSTYRV VSVLTVLHQDWLNGKEYKCKVSNKALPAPIEKTISKAKGQPREPQVYTLPPSREEMTKNQV SLSCAVKGFYPSDIAVEWESNGQPENNYKTTPPVLDSDGSFFLVSKLTVDKSRWQQGNVFS CSVMHEALHNRFTQKSLSLSPG (SEQ ID NO:40) or QVQLVQSGAEVKKPGASVKVSCKASGYTFTDYYMHWVRQAPGQGLEWMGWINPNSGDT NYAQKFQGRVTMTRDTSISTAYMELSRLRSDDTAVYYCARNTGAYSFGYWGQGTLVTVSS ASTKGPSVFPLAPSSKSTSGGTAALGCLVKDYFPEPVTVSWNSGALTSGVHTFPAVLQSSGL YSLSSVVTVPSSSLGTQTYICNVNHKPSNTKVDKKVEPKSCDKTHTCPPCPAPELLGGPSVFL FPPKPKDTLMISRTPEVTCVVVDVSHEDPEVKFNWYVDGVEVHNAKTKPREEQYNSTYRV VSVLTVLHQDWLNGKEYKCKVSNKALPAPIEKTISKAKGQPREPQVYTLPPSREEMTKNQV SLSCAVKGFYPSDIAVEWESNGQPENNYKTTPPVLDSDGSFFLVSKLTVDKSRWQQGNVFS CSVMHEALHNRFTQKSLSLSPGK (SEQ ID NO:41), and the fifth polypeptide comprises the amino acid sequence of DIQMTQSPSSVSASVGDRVTITCRASQGISSWLAWYQQKPGKAPKLLIYAASSLQSGVPSRF SGSGSGTDFTLTISSLQPEDFATYYCQQAYGFPFTFGPGTKVDIKRTVAAPSVFIFPPSDEQLK SGTASVVCLLNNFYPREAKVQWKVDNALQSGNSQESVTEQDSKDSTYSLSSTLTLSKADYE KHKVYACEVTHQGLSSPVTKSFNRGEC (SEQ ID NO:42). In some embodiments, the first polypeptide comprises the amino acid sequence of QVQLQESGPGLVKPSGTLSLTCAVSGGSISSSNWWSWVRQPPGKGLEWIGEIYHDGSTDYF PSLKSRVTISVDKSKNQFSLKLSSVTAADTAVYYCARDNWGFDYWGQGTLVTVSSASTKG VALHRPDVYLLPPAREQLNLRESATISCAVTGFSPADVFVQWMQRGQPLSPEKYVTSAPMP EPQAPGRYFAVSILTVSEEEWNTGETYTCVVAHEALPNRVTERTVDKGGGGSGGGGSQVQ LVQSGAEVKKPGASVKVSCKASGYTFTDYYMHWVRQAPGQGLEWMGWINPNSGDTNYA QKFQGRVTMTRDTSISTAYMELSRLRSDDTAVYYCARNTGAYSFGYWGQGTLVTVSSAST KGPSVFPLAPSSKSTSGGTAALGCLVKDYFPEPVTVSWNSGALTSGVHTFPAVLQSSGLYSL SSVVTVPSSSLGTQTYICNVNHKPSNTKVDKKVEPKSCDKTHTCPPCPAPELLGGPSVFLFPP KPKDTLMISRTPEVTCVVVDVSHEDPEVKFNWYVDGVEVHNAKTKPREEQYNSTYRVVSV LTVLHQDWLNGKEYKCKVSNKALPAPIEKTISKAKGQPREPQVYTLPPSREEMTKNQVSLW CLVKGFYPSDIAVEWESNGQPENNYKTTPPVLDSDGSFFLYSKLTVDKSRWQQGNVFSCSV MHEALHNHYTQKSLSLSPG (SEQ ID NO:37) or QVQLQESGPGLVKPSGTLSLTCAVSGGSISSSNWWSWVRQPPGKGLEWIGEIYHDGSTDYF PSLKSRVTISVDKSKNQFSLKLSSVTAADTAVYYCARDNWGFDYWGQGTLVTVSSASTKG VALHRPDVYLLPPAREQLNLRESATISCAVTGFSPADVFVQWMQRGQPLSPEKYVTSAPMP EPQAPGRYFAVSILTVSEEEWNTGETYTCVVAHEALPNRVTERTVDKGGGGSGGGGSQVQ LVQSGAEVKKPGASVKVSCKASGYTFTDYYMHWVRQAPGQGLEWMGWINPNSGDTNYA QKFQGRVTMTRDTSISTAYMELSRLRSDDTAVYYCARNTGAYSFGYWGQGTLVTVSSAST KGPSVFPLAPSSKSTSGGTAALGCLVKDYFPEPVTVSWNSGALTSGVHTFPAVLQSSGLYSL SSVVTVPSSSLGTQTYICNVNHKPSNTKVDKKVEPKSCDKTHTCPPCPAPELLGGPSVFLFPP KPKDTLMISRTPEVTCVVVDVSHEDPEVKFNWYVDGVEVHNAKTKPREEQYNSTYRVVSV LTVLHQDWLNGKEYKCKVSNKALPAPIEKTISKAKGQPREPQVYTLPPSREEMTKNQVSLW CLVKGFYPSDIAVEWESNGQPENNYKTTPPVLDSDGSFFLYSKLTVDKSRWQQGNVFSCSV MHEALHNHYTQKSLSLSPGK (SEQ ID NO:38), the second polypeptide comprises the amino acid sequence of DIVLTQSPGTLSLSPGERATLSCRASQSVSSSYLAWYQQKPGQAPRLLISGASSRATGIPDRES GSGSGTDFTLTISRLEPEDFAVYYCQQYGSSHRTFGQGTKVEIKRTVALHRPDVYLLPPARE QLNLRESATIWCLVTGFSPADVFVQWMQRGQPLSPEKYVTSAPMPEPQAPGRYFAHSILTV SEEEWNTGETYTCVVAHEALPNRVTERTVDKSTGES (SEQ ID NO:39), and the third polypeptide comprises the amino acid sequence of DIQMTQSPSSVSASVGDRVTITCRASQGISSWLAWYQQKPGKAPKLLIYAASSLQSGVPSRF SGSGSGTDFTLTISSLQPEDFATYYCQQAYGFPFTFGPGTKVDIKRTVAAPSVFIFPPSDEQLK SGTASVVCLLNNFYPREAKVQWKVDNALQSGNSQESVTEQDSKDSTYSLSSTLTLSKADYE KHKVYACEVTHQGLSSPVTKSFNRGEC (SEQ ID NO:42). In some embodiments, the first polypeptide comprises the amino acid sequence of QVQLQESGPGLVKPSGTLSLTCAVSGGSISSSNWWSWVRQPPGKGLEWIGEIYHDGSTDYF PSLKSRVTISVDKSKNQFSLKLSSVTAADTAVYYCARDNWGFDYWGQGTLVTVSSASTKG VALHRPDVYLLPPAREQLNLRESATISCAVTGFSPADVFVQWMQRGQPLSPEKYVTSAPMP EPQAPGRYFAVSILTVSEEEWNTGETYTCVVAHEALPNRVTERTVDKSCGGGGSGGGGSQV QLVQSGAEVKKPGASVKVSCKASGYTFTDYYMHWVRQAPGQGLEWMGWINPNSGDTNY AQKFQGRVTMTRDTSISTAYMELSRLRSDDTAVYYCARNTGAYSFGYWGQGTLVTVSSAS TKGPSVFPLAPSSKSTSGGTAALGCLVKDYFPEPVTVSWNSGALTSGVHTFPAVLQSSGLYS LSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAKGQPREPQVYTLPPSREEMTKNQVSL WCLVKGFYPSDIAVEWESNGQPENNYKTTPPVLDSDGSFFLYSKLTVDKSRWQQGNVFSCS VMHEALHNHYTQKSLSLSPG (SEQ ID NO:43) or QVQLQESGPGLVKPSGTLSLTCAVSGGSISSSNWWSWVRQPPGKGLEWIGEIYHDGSTDYF PSLKSRVTISVDKSKNQFSLKLSSVTAADTAVYYCARDNWGFDYWGQGTLVTVSSASTKG VALHRPDVYLLPPAREQLNLRESATISCAVTGFSPADVFVQWMQRGQPLSPEKYVTSAPMP EPQAPGRYFAVSILTVSEEEWNTGETYTCVVAHEALPNRVTERTVDKSCGGGGSGGGGSQV QLVQSGAEVKKPGASVKVSCKASGYTFTDYYMHWVRQAPGQGLEWMGWINPNSGDTNY AQKFQGRVTMTRDTSISTAYMELSRLRSDDTAVYYCARNTGAYSFGYWGQGTLVTVSSAS TKGPSVFPLAPSSKSTSGGTAALGCLVKDYFPEPVTVSWNSGALTSGVHTFPAVLQSSGLYS LSSVVTVPSSSLGTQTYICNVNHKPSNTKVDKKVEPKSCDKTHTCPPCPAPELLGGPSVFLFP PKPKDTLMISRTPEVTCVVVDVSHEDPEVKFNWYVDGVEVHNAKTKPREEQYNSTYRVVS VLTVLHQDWLNGKEYKCKVSNKALPAPIEKTISKAKGQPREPQVYTLPPSREEMTKNQVSL WCLVKGFYPSDIAVEWESNGQPENNYKTTPPVLDSDGSFFLYSKLTVDKSRWQQGNVFSCS VMHEALHNHYTQKSLSLSPGK (SEQ ID NO:44), the second polypeptide comprises the amino acid sequence of DIVLTQSPGTLSLSPGERATLSCRASQSVSSSYLAWYQQKPGQAPRLLISGASSRATGIPDRES GSGSGTDFTLTISRLEPEDFAVYYCQQYGSSHRTFGQGTKVEIKRTVALHRPDVYLLPPARE QLNLRESATIWCLVTGFSPADVFVQWMQRGQPLSPEKYVTSAPMPEPQAPGRYFAHSILTV SEEEWNTGETYTCVVAHEALPNRVTERTVDKSTGEC (SEQ ID NO:45), and the third polypeptide comprises the amino acid sequence of SEQ ID NO:42. In some embodiments, the first polypeptide comprises the amino acid sequence of SEQ ID NO:37 or 38, the second polypeptide comprises the amino acid sequence of SEQ ID NO:39, the third polypeptide comprises the amino acid sequence of SEQ ID NO:42, the fourth polypeptide comprises the amino acid sequence of SEQ ID NO: 40 or 41, and the fifth polypeptide comprises the amino acid sequence of SEQ ID NO:42. In some embodiments, the first polypeptide comprises the amino acid sequence of SEQ ID NO:43 or 44, the second polypeptide comprises the amino acid sequence of SEQ ID NO:45, the third polypeptide comprises the amino acid sequence of SEQ ID NO:42, the fourth polypeptide comprises the amino acid sequence of SEQ ID NO:40 or 41, and the fifth polypeptide comprises the amino acid sequence of SEQ ID NO:42.

In some embodiments, the multispecific antibodies of the present disclosure comprise:
a) a first arm comprising:
   i) a first polypeptide that comprises, in an N-terminal to C-terminal direction, a structure represented by the formula:

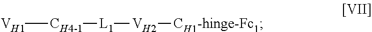

ii) a second polypeptide that comprises, in the N-terminal to C-terminal direction, a structure represented by the formula:

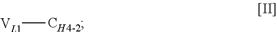

and
iii) a third polypeptide that comprises, in the N-terminal to C-terminal direction, a structure represented by the formula:

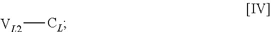

and
b) a second arm comprising:
   iv) a fourth polypeptide that comprises, in the N-terminal to C-terminal direction, a structure represented by the formula:

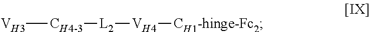

v) a fifth polypeptide that comprises, in the N-terminal to C-terminal direction, a structure represented by the formula:

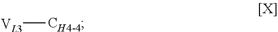

and
vi) a sixth polypeptide that comprises, in the N-terminal to C-terminal direction, a structure represented by the formula:

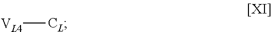

wherein:
   V_{H1} is a first heavy chain variable (VH) domain;
   V_{H2} is a second VH domain;
   V_{H3} is a third VH domain;
   V_{H4} is a fourth VH domain;
   V_{L1} is a first light chain variable (VL) domain;
   V_{L2} is a second VL domain;
   V_{L3} is a third VL domain;
   V_{L4} is a fourth VL domain;
   L₁ is a first linker sequence;
   L₂ is a second linker sequence;
   C_{H4-1} is a first antibody constant heavy chain 4 (CH4) domain;
   C_{H4-2} is a second antibody CH4 domain;
   C_{H4-3} is a third antibody CH4 domain;
   C_{H4-4} is a fourth antibody CH4 domain;
   C_{H1} is an antibody constant heavy chain 1 (CH1) domain;
   hinge is an antibody hinge region;
   Fc₁ is a first antibody Fc region;
   Fc₂ is a second antibody Fc region; and
   C_{L} is an antibody light chain constant (CL) domain;
      wherein the first VH domain and the first VL domain form a first antigen binding site that specifically binds a first antigen, the second VH domain and the second VL domain form a second antigen binding site that specifically binds a second antigen, the third VH domain and the third VL domain form a third antigen binding site that specifically binds a third antigen, and the fourth VH domain and the fourth VL domain form a fourth antigen binding site that specifically binds a fourth antigen.

In some embodiments, the first and/or second antibody CH4 domains comprise at least one amino acid substitution that promotes association of the first and second antibody CH4 domains. In some embodiments, the third and/or fourth antibody CH4 domains comprise at least one amino acid substitution that promotes association of the third and fourth antibody CH4 domains. In some embodiments, the first and/or second antibody Fc regions comprise at least one amino acid substitution that promotes association of the first and second antibody Fc regions.

An exemplary configuration of a multispecific binding protein is shown in FIG. 9D. In some embodiments, the second and third; first and second; first and third; first and fourth; second and fourth; third and fourth; first, second, and third; first, third, and fourth; second, third, and fourth; or first, second, and fourth antigens are the same. In some embodiments, the second and third; first and second; first and third; first and fourth; second and fourth; third and fourth; first, second, and third; first, third, and fourth; second, third, and fourth; or first, second, and fourth antigen binding sites are the same, e.g., comprising the same VH and VL domain sequences. In some embodiments, the first, second, third, and fourth antigens are different. In some embodiments, the first, second, third, and fourth antigen binding sites are different.

A variety of antibody constant domains useful for heterodimerization between heavy and light chains of a common antigen binding site (e.g., a heterodimerization pair) are contemplated for use herein. In some embodiments, the antibody CH4 domains of a heterodimerization pair are human CH4 domains.

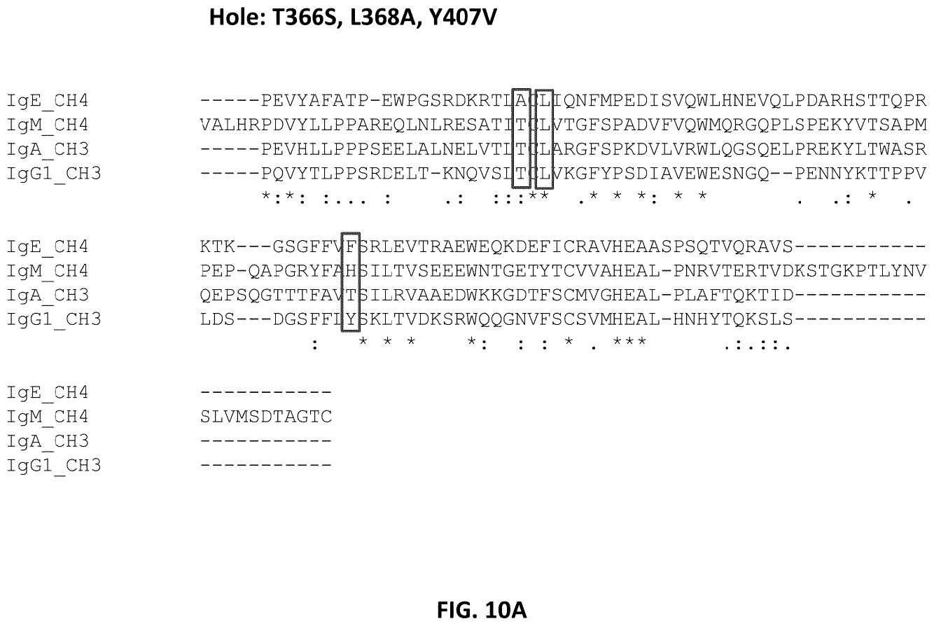

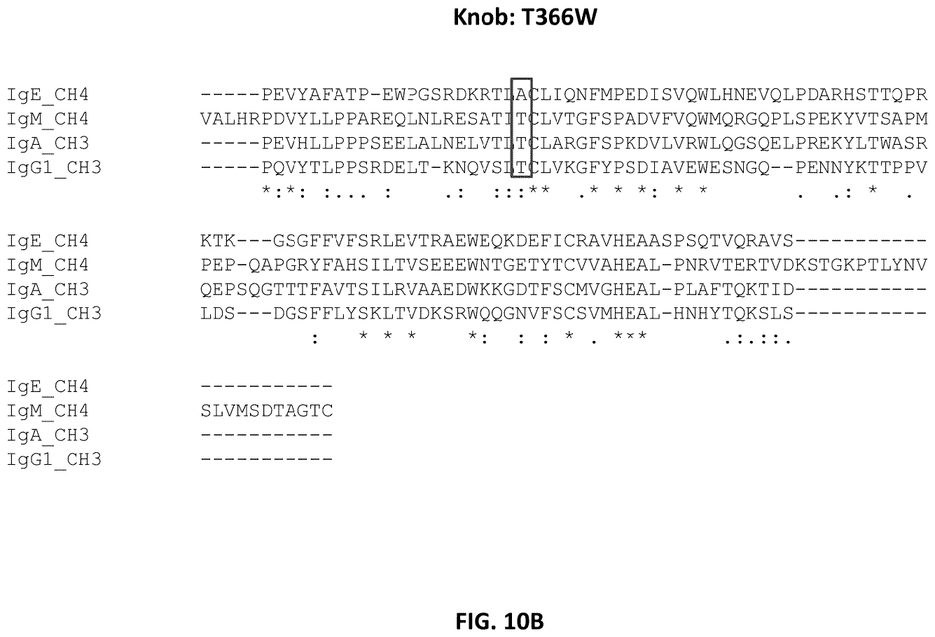

In some embodiments, the first and second antibody CH4 domains of a heterodimerization pair are IgM CH4 domains, e.g., human IgM CH4 domains. In some embodiments, the first and/or second antibody CH4 domains each comprise at least one amino acid substitution relative to the amino acid sequence of an IgM CH4 domain (e.g., a human IgM CH4 domain) that promotes association of the first and second antibody CH4 domains. In some embodiments, the first and/or second antibody CH4 domains each comprise at least one amino acid substitution relative to the amino acid sequence of VALHRPDVYLLPPAREQLNLRESATITCLVTGFSPADVFVQWMQRGQPLSPEKYVTSAPMP EPQAPGRYFAHSILTVSEEEWNTGETYTCVVAHEALPNRVTERTVDKS (SEQ ID NO:1) that promotes association of the first and second antibody CH4 domains. In some embodiments, the first IgM CH4 domain comprises one or more hole-forming substitutions, and the second IgM CH4 domain comprises one or more knob-forming substitutions. In some embodiments, the first IgM CH4 domain comprises one or more knob-forming substitutions, and the second IgM CH4 domain comprises one or more hole-forming substitutions. In some embodiments, an IgM CH4 domain comprising one or more knob-forming substitutions comprises an IgM CH4 domain (e.g., a human IgM CH4 domain) comprising a substitution equivalent to T366W (numbering based on human IgG1 according to EU index). In some embodiments, an IgM CH4 domain comprising one or more knob-forming substitutions comprises the amino acid sequence of VALHRPDVYLLPPAREQLNLRESATIWCLVTGFSPADVFVQWMQRGQPLSPEKYVTSAPMP EPQAPGRYFAHSILTVSEEEWNTGETYTCVVAHEALPNRVTERTVDKS (SEQ ID NO:3). In some embodiments, an IgM CH4 domain comprising one or more hole-forming substitutions comprises an IgM CH4 domain (e.g., a human IgM CH4 domain) comprising substitutions equivalent to T366S, L368A, and Y407V (numbering based on human IgG1 according to EU index). In some embodiments, an IgM CH4 domain comprising one or more hole-forming substitutions comprises the amino acid sequence of VALHRPDVYLLPPAREQLNLRESATISCAVTGFSPADVFVQWMQRGQPLSPEKYVTSAPMP EPQAPGRYFAVSILTVSEEEWNTGETYTCVVAHEALPNRVTERTVDKS (SEQ ID NO:2). In some embodiments, the first and/or second IgM CH4 domains comprise one or more engineered positively or negatively charged residues that promotes electrostatic association between the first and second IgM CH4 domains. Positions of IgM corresponding to knob- and hole-forming residues of IgG1 are shown in FIGS. 10A & 10B.

In some embodiments, the first and second antibody CH4 domains of a heterodimerization pair are IgE CH4 domains, e.g., human IgE CH4 domains. In some embodiments, the first and/or second antibody CH4 domains each comprise at least one amino acid substitution relative to the amino acid sequence of an IgE CH4 domain (e.g., a human IgE CH4 domain) that promotes association of the first and second antibody CH4 domains. In some embodiments, the first and/or second antibody CH4 domains each comprise at least one amino acid substitution relative to the amino acid sequence of PEVYAFATPEWPGSRDKRTLACLIQNFMPEDISVQWLHNEVQLPDARHSTTQPRKTK GSGFFVFSRLEVTRAEWEQKDEFICRAVHEAASPSQTVQRAVS (SEQ ID NO:4) that promotes association of the first and second antibody CH4 domains. In some embodiments, the first IgE CH4 domain comprises one or more hole-forming substitutions, and the second IgE CH4 domain comprises one or more knob-forming substitutions. In some embodiments, the first IgE CH4 domain comprises one or more knob-forming substitutions, and the second IgE CH4 domain comprises one or more hole-forming substitutions. In some embodiments, an IgE CH4 domain comprising one or more knob-forming substitutions comprises an IgE CH4 domain (e.g., a human IgE CH4 domain) comprising a substitution equivalent to T366W (numbering based on human IgG1 according to EU index). In some embodiments, an IgE CH4 domain comprising one or more knob-forming substitutions comprises the amino acid sequence of PEVYAFATPEWPGSRDKRTLWCLIQNFMPEDISVQWLHNEVQLPDARHSTTQPRKTK GSGFFVFSRLEVTRAEWEQKDEFICRAVHEAASPSQTVQRAVS (SEQ ID NO:6). In some embodiments, an IgE CH4 domain comprising one or more hole-forming substitutions comprises an IgE CH4 domain (e.g., a human IgE CH4 domain) comprising substitutions equivalent to T366S, L368A, and Y407V (numbering based on human IgG1 according to EU index). In some embodiments, an IgE CH4 domain comprising one or more hole-forming substitutions comprises the amino acid sequence of PEVYAFATPEWPGSRDKRTLSCAIQNFMPEDISVQWLHNEVQLPDARHSTTQPRKTK GSGFFVVSRLEVTRAEWEQKDEFICRAVHEAASPSQTVQRAVS (SEQ ID NO:5). In some embodiments, the first and/or second IgE CH4 domains comprise one or more engineered positively or negatively charged residues that promotes electrostatic association between the first and second IgE CH4 domains. Positions of IgE corresponding to knob- and hole-forming residues of IgG1 are shown in FIGS. 10A & 10B.

In some embodiments, a multispecific antibody of the present disclosure comprises a pair of antibody CH3 domains for heterodimerization in place of a pair of antibody CH4 domains as described herein.

In some embodiments, the first and second antibody CH3 domains of a heterodimerization pair are IgA CH3 domains, e.g., human IgA CH3 domains. In some embodiments, the first and/or second antibody CH3 domains each comprise at least one amino acid substitution relative to the amino acid sequence of an IgA CH3 domain (e.g., a human IgA CH3 domain) that promotes association of the first and second antibody CH3 domains. In some embodiments, the first and/or second antibody CH3 domains each comprise at least one amino acid substitution relative to the amino acid sequence of PEVHLLPPPSEELALNELVTLTCLARGFSPKDVLVRWLQGSQELPREKYLTWASR QEPSQGTTTFAVTSILRVAAEDWKKGDTFSCMVGHEALPLAFTQKTID (SEQ ID NO:7) that promotes association of the first and second antibody CH3 domains. In some embodiments, the first IgA CH3 domain comprises one or more hole-forming substitutions, and the second IgA CH3 domain comprises one or more knob-forming substitutions. In some embodiments, the first IgA CH3 domain comprises one or more knob-forming substitutions, and the second IgA CH3 domain comprises one or more hole-forming substitutions. In some embodiments, an IgA CH3 domain comprising one or more knob-forming substitutions comprises an IgA CH3 domain (e.g., a human IgA CH3 domain) comprising a substitution equivalent to T366W (numbering based on human IgG1 according to EU index). In some embodiments, an IgA CH3 domain comprising one or more knob-forming substitutions comprises the amino acid sequence of PEVHLLPPPSEELALNELVTLWCLARGFSPKDVLVRWLQGSQELPREKYLTWASR QEPSQGTTTFAVTSILRVAAEDWKKGDTFSCMVGHEALPLAFTQKTID (SEQ ID NO:9). In some embodiments, an IgA CH3 domain comprising one or more hole-forming substitutions comprises an IgA CH3 domain (e.g., a human IgA CH3 domain) comprising substitutions equivalent to T366S, L368A, and Y407V (numbering based on human IgG1 according to EU index). In some embodiments, an IgA CH3 domain comprising one or more hole-forming substitutions comprises the amino acid sequence of PEVHLLPPPSEELALNELVTLSCAARGFSPKDVLVRWLQGSQELPREKYLTWASR QEPSQGTTTFAVVSILRVAAEDWKKGDTFSCMVGHEALPLAFTQKTID (SEQ ID NO:8). In some embodiments, the first and/or second IgA CH3 domains comprise one or more engineered positively or negatively charged residues that promotes electrostatic association between the first and second IgA CH3 domains. Positions of IgA corresponding to knob- and hole-forming residues of IgG1 are shown in FIGS. 10A & 10B.

In some embodiments, the first and second antibody CH3 domains of a heterodimerization pair are IgG CH3 domains, e.g., human IgG CH3 domains. In some embodiments, the first and second antibody CH3 domains of a heterodimerization pair are IgG1, IgG2, IgG3, or IgG4 CH3 domains, e.g., human IgG1, IgG2, IgG3, or IgG4 CH3 domains. In some embodiments, the first and/or second antibody CH3 domains each comprise at least one amino acid substitution relative to the amino acid sequence of an IgG CH3 domain (e.g., a human IgG CH3 domain) that promotes association of the first and second antibody CH3 domains. In some embodiments, the first and/or second antibody CH3 domains each comprise at least one amino acid substitution relative to the amino acid sequence of PQVYTLPPSRDELTKNQVSLTCLVKGFYPSDIAVEWESNGQPENNYKTTPPVLDS DGSFFLYSKLTVDKSRWQQGNVFSCSVMHEALHNHYTQKSLS (SEQ ID NO:10) that promotes association of the first and second antibody CH3 domains. In some embodiments, the first IgG CH3 domain comprises one or more hole-forming substitutions, and the second IgG CH3 domain comprises one or more knob-forming substitutions. In some embodiments, the first IgG CH3 domain comprises one or more knob-forming substitutions, and the second IgG CH3 domain comprises one or more hole-forming substitutions. In some embodiments, an IgG CH3 domain comprising one or more knob-forming substitutions comprises an IgG CH3 domain (e.g., a human IgG CH3 domain) comprising a substitution equivalent to T366W (numbering based on human IgG1 according to EU index). In some embodiments, an IgG CH3 domain comprising one or more knob-forming substitutions comprises the amino acid sequence of PQVYTLPPSRDELTKNQVSLWCLVKGFYPSDIAVEWESNGQPENNYKTTPPVLDS DGSFFLYSKLTVDKSRWQQGNVFSCSVMHEALHNHYTQKSLS (SEQ ID NO:12). In some embodiments, an IgG CH3 domain comprising one or more hole-forming substitutions comprises an IgG CH3 domain (e.g., a human IgG CH3 domain) comprising substitutions equivalent to T366S, L368A, and Y407V (numbering based on human IgG1 according to EU index). In some embodiments, an IgG CH3 domain comprising one or more hole-forming substitutions comprises the amino acid sequence of PQVYTLPPSRDELTKNQVSLSCAVKGFYPSDIAVEWESNGQPENNYKTTPPVLDS DGSFFLVSKLTVDKSRWQQGNVFSCSVMHEALHNHYTQKSLS (SEQ ID NO:11). In some embodiments, the first and/or second IgG CH3 domains comprise one or more engineered positively or negatively charged residues that promotes electrostatic association between the first and second IgG CH3 domains.

In some embodiments, an Fc, Fc region, or Fc domain refers to the C-terminal region of an antibody heavy chain that contains at least a portion of the constant region. The term includes native sequence Fc regions and variant Fc regions. An Fc can refer to the last two constant region immunoglobulin domains (e.g., CH2 and CH3) of IgA, IgD, and IgG, the last three constant region immunoglobulin domains of IgE and IgM, and optionally, all or a portion of the flexible hinge N-terminal to these domains. For IgA and IgM, Fc may include the J chain. An IgG Fc region comprises an IgG CH2 and an IgG CH3 domain and in some cases, inclusive of the hinge. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. Human IgG Fc domains are of particular use in the present disclosure, and can be the Fc domain from human IgG1, IgG2 or IgG4.

In some embodiments, an antibody of the present disclosure comprises an Fc region. An antibody may be of any class or subclass, including IgG and subclasses thereof (IgG1, IgG2, IgG3, IgG4), IgM, IgE, IgA, and IgD. An immunoglobulin Fc region of the molecule that causes targeted phagocytosis may have important role in the process by engaging Fc receptors and inducing additional phagocytosis. In some embodiments, the molecule has a modified Fc region that has reduced ADCC activity as compared to a wild type human IgG1 (e.g., comprising one or more mutations reducing effector function as described herein).

In some embodiments, an antibody of the present disclosure comprises an Fc region wherein a carbohydrate structure attached to the Fc region has reduced fucose or lacks fucose, e.g., at least one or two of the heavy chains of the antibody is non-fucosylated or comprise reduced fucosylation. In some embodiments, provided herein is a composition comprising an antibody of the present disclosure that comprises an Fc region wherein a carbohydrate structure attached to the Fc region has reduced fucose or lacks fucose, e.g., at least one or two of the heavy chains of the antibody is/are non-fucosylated or comprise(s) reduced fucosylation. In some embodiments, less than 50% of the N-glycoside-linked carbohydrate chains in the composition contain a fucose residue. In some embodiments, substantially none of the N-glycoside-linked carbohydrate chains contain a fucose residue. In some embodiments, an antibody with reduced fucose or lacking fucose has improved ADCC function.

In some embodiments, the antibody further comprises an Fc region. In some embodiments, the Fc region is a human IgG Fc region. In some embodiments, the Fc region is a human IgG1 or human IgG4 Fc region. In some embodiments, the Fc region is a human IgG1 Fc region comprising S239D and 1332E substitutions, according to EU numbering. In some embodiments, the Fc region is a human IgG1 Fc region comprising S239D, A330L, and I332E substitutions, according to EU numbering. In some embodiments, the Fc region is a human IgG1 Fc region comprising G236A, S239D, A330L, and I332E substitutions, according to EU numbering. In some embodiments, the Fc region is a human IgG4 Fc region comprising an S228P substitution, according to EU numbering.

In other embodiments, an antibody of the present disclosure (e.g., an IgG1 antibody) or composition comprising an antibody of the present disclosure (e.g., an IgG1 antibody) comprises wild-type glycosylation of the Fc region. In some embodiments, provided herein are fucosylated antibodies of the present disclosure (e.g., an IgG1 antibody) or compositions comprising a fucosylated antibody of the present disclosure (e.g., an IgG1 antibody),

Fucosylation or fucosylated antibodies can refer to the presence of fucose residues within the oligosaccharides attached to the peptide backbone of an antibody. Specifically, a fucosylated antibody comprises a (1,6)-linked fucose at the innermost N-acetylglucosamine (GlcNAc) residue in one or both of the N-linked oligosaccharides attached to the antibody Fc region, e.g., at position Asn 297 of the human IgG1 Fc region (EU numbering of Fc region residues). Asn297 may also be located about +3 amino acids upstream or downstream of position 297, i.e., between positions 294 and 300, due to minor sequence variations in immunoglobulins, Non-fucosylated or fucose-deficient antibodies have reduced fucose relative to the amount of fucose on the same antibody produced in a cell line. Antibody fucosylation can be measured, e.g., in an N-glycosidase F treated antibody composition assessed by matrix-assisted laser desorption-ionization time-of-flight mass spectrometry (MALDI TOF MS).

In some embodiments, the Fc region comprises one or more mutations that reduce or eliminate fucosylation, e.g., a substitution at Asn 297 of the human IgG1 Fe region (EU numbering of Fc region residues). Optionally, the Fc region further comprises one or more amino acid substitutions therein which further improve ADCC, for example, substitutions at positions 298, 333, and/or 334 of the Fc region (Eu numbering of residues). Examples of publications related to “defucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; Okazaki et al. J. Mol. Biol. 336:1239-1249 (2004); Yamane-Ohnuki et al. Biotech. Bioeng. 87:614 (2004).

In some embodiments, the afucosylated or non-fucosylated antibody is produced in a cell line with a genetic modification that results in an afucosylated or non-fucosylated antibody. Examples of cell lines producing afucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (Yamane-Ohnuki et al. Biotech. Bioeng. 87:614 (2004)), cells overexpressing β1,4-N-acetylglucosaminyltransferase III (GnT-III) and Golgi μ-mannosidase II (ManII), and cells with a knockout in the mannosyl-glycoprotein 2-beta-N-acetylglucosaminyltranferase (MGAT1; see Byrne, G. et al. (2018) PLOS Biol. 16:e2005817).

In some embodiments, the afucosylated or non-fucosylated antibody is produced in a cell line treated with an inhibitor of glycoprocessing enzyme(s), such as kifunensine, which is an inhibitor of mannosidase I (see, e.g., Elbein, A. D. et al. (1990) J. Biol. Chem. 265:15599-15605). For example, cells can be centrifuged and resuspended in growth medium comprising kifunensine (e.g., at 250 μg/mL), then cultured and used for antibody production.

Methods for making bispecific antibodies are known in the art. One well-established approach for making multispecific antibodies is the “knobs-into-holes” or “protuberance-into-cavity” approach. See e.g., U.S. Pat. No. 5,731,168. Two immunoglobulin polypeptides (e.g., heavy chain polypeptides) each comprise an interface; an interface of one immunoglobulin polypeptide interacts with a corresponding or cognate interface on the other immunoglobulin polypeptide, thereby allowing the two immunoglobulin polypeptides to associate. In some embodiments, interfaces may be engineered such that a “knob” or “protuberance” located in the interface of one immunoglobulin polypeptide corresponds with a cognate “hole” or “cavity” located in the interface of the other immunoglobulin polypeptide. In some embodiments, a knob may be constructed by replacing a small amino acid side chain with a larger side chain. In some embodiments, a hole may be constructed by replacing a large amino acid side chain with a smaller side chain. Knobs or holes may exist in the original interface, or they may be introduced synthetically. Polynucleotides encoding modified immunoglobulin polypeptides with one or more corresponding knob- or hole-forming mutations may be expressed and purified using standard recombinant techniques and cell systems known in the art. See, e.g., U.S. Pat. Nos. 5,731,168; 5,807,706; 5,821,333; 7,642,228; 7,695,936; 8,216,805; 8,679,785; 8,844,834; U.S. Pub. No. 2013/0089553; Spiess et al., Nature Biotechnology 31:753-758, 2013; and Ridgway and Carter (1996) Protein Eng. 9:617-621. Modified immunoglobulin polypeptides may be produced using prokaryotic host cells, such as E. coli, or eukaryotic host cells, such as mammalian cells (e.g., CHO cells) or yeast cells. Corresponding knob- and hole-bearing immunoglobulin polypeptides may be expressed in host cells in co-culture and purified together as a heteromultimer, or they may be expressed in single cultures, separately purified, and assembled in vitro. Exemplary cognate knob and hole mutations are provided below (numbering according to EU index). EU numbering as used herein is known in the art; see, e.g., IMGT resources at www.imgt.org/IMGTScientificChart/Numbering/Hu_IGHGnber.html and www.imgt.org/IMGTScientificChart/Numbering/Hu_IGKCnber.html. As used herein, an “antibody arm” may refer to the pairing between an antibody heavy chain and an antibody light chain, wherein the variable domains of the heavy and light chains form an antigen binding site that binds a target antigen.

| | | | | | | | | |
|---|---|---|---|---|---|---|---|---|
| Fc region 1 | Y407T | Y407A | F405A | T394S | T366S | T394W | T394S | T366W |
| | | | | | L368A | Y407T | Y407A | T394S |
| | | | | | Y407V | | | |
| Fc region 2 | T366Y | T366W | T394W | F405W | T366W | T366Y | T366W | F405W |
| | | | | | | F405A | F405W | Y407A |

In some embodiments, the first antibody Fc region of a multispecific antibody of the present disclosure comprises one or more hole-forming substitutions, and the second antibody Fc region comprises one or more knob-forming substitutions. In some embodiments, the first antibody Fc region of a multispecific antibody of the present disclosure comprises one or more knob-forming substitutions, and the second antibody Fc region comprises one or more hole-forming substitutions. Exemplary sets of knob- and hole-forming substitutions are set forth in the table supra. In some embodiments, the hole-forming substitutions comprise T366S, L368A, and Y407V (or an equivalent thereof), and the knob-forming substitution is T366W (or an equivalent thereof).

In some embodiments, multispecific (e.g., bispecific) antibodies further comprise one or more mutations on only one of the antibody arms to improve heavy chain/light chain pairing. For example, amino acid substitutions can be used to replace a native disulfide bond in the CH1-CL interface of one antibody arm with an engineered disulfide bond. See, e.g., Mazor, Y. et al. (2015) MAbs 7:377-389 and EP3452089A2. In some embodiments, the multispecific or bispecific antibody comprises two antibody light chains and two antibody heavy chains, wherein only one of the antibody heavy chains comprises amino acid substitutions F126C and C220V, and only the corresponding or cognate light chain comprises amino acid substitutions S121C and C214V, according to EU numbering.

Multispecific (e.g., bispecific) antibodies also include cross-linked or “heteroconjugate” antibodies. Techniques for generating bispecific antibodies from antibody fragments have also been described in the literature. For example, bispecific antibodies can be prepared using chemical linkage. In some embodiments, a bispecific antibody comprises a first IgG antibody comprising the first antigen binding site covalently linked to a second IgG antibody comprising the second antigen binding site.

In some embodiments, multispecific (e.g., bispecific) antibodies further comprise one or more mutations on only one of the antibody arms to reduce binding affinity for Protein A. See, e.g., Ollier, R. et al. (2019) MAbs 11:1464-1478 and AU2018204314. In some embodiments, the multispecific or bispecific antibody comprises two antibody light chains and two antibody heavy chains, wherein only one of the antibody heavy chains comprises amino acid substitutions H435R and Y436F, according to EU numbering.

In some embodiments, the monospecific or multispecific (e.g., bispecific) antibodies further comprise one or more mutations to reduce effector function, e.g., to reduce or eliminate binding of the Fc region to an Fc receptor. In some embodiments, the antibody comprises two antibody Fc regions, wherein the antibody Fc regions comprise an amino acid substitution at one or more of positions 234, 235, and 237, according to EU numbering. In some embodiments, the antibody comprises two antibody Fc regions, wherein the antibody Fc regions comprise L234A, L235E, and G237A substitutions, according to EU numbering.

In some embodiments, the monospecific or multispecific (e.g., bispecific) antibodies comprise two antibody heavy chains and two antibody light chains, wherein the VH domain of the first antibody heavy chain forms an antigen binding site with the VL domain of the first antibody light chain, wherein the VH domain of the second antibody heavy chain forms an antigen binding site with the VL domain of the second antibody light chain, wherein the first antibody heavy chain comprises F126C, C220V, and T366W substitutions, wherein the first antibody light chain comprises S121C and C214V substitutions, and wherein the second antibody heavy chain comprises T366S, L368A, Y407V, H435R, and Y436F substitutions, according to EU numbering. In some embodiments, the first and second antibody heavy chains further comprise L234A, L235E, and G237A substitutions, according to EU numbering. In some embodiments, the first and second antibody heavy chains comprise human IgG1 Fc domains.

In some embodiments, antibody and immunoglobulin are used interchangeably and herein are used in the broadest sense and encompass various antibody structures, including but not limited to monoclonal antibodies (e.g., full length or intact monoclonal antibodies), polyclonal antibodies, multispecific antibodies (e.g., bispecific antibodies), antibody fragments and single domain antibody (as described in greater detail herein), so long as they exhibit the desired antigen binding activity.

In some embodiments, antibodies (immunoglobulins) refer to a protein having a structure substantially similar to a native antibody structure, or a protein having heavy and light chain variable regions having structures substantially similar to native heavy and light chain variable region structures. Native antibodies refer to naturally occurring immunoglobulin molecules with varying structures. For example, native immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The subunit structures and three-dimensional configurations of the different classes of immunoglobulins are well known and described generally, for example, in Abbas et al., 2000, Cellular and Mol, and Kindt et al., Kuby Immunology, 6th ed., W. H. Freeman and Co., page 91 (2007). Antibodies (immunoglobulins) are assigned to different classes, depending on the amino acid sequences of the heavy chain constant domains. There are five major classes of antibodies: α (IgA), δ (IgD), ϵ (IgE), γ (IgG), or μ (IgM), some of which may be further divided into subtypes, e.g., γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

In some embodiments, one or more antigen binding sites of the present disclosure is/are humanized.

In some embodiments, a multispecific antibody of the present disclosure further comprises a tag, e.g., for affinity purification. In some embodiments, the tag is a polyhistidine tag.

In some embodiments, a multispecific antibody of the present disclosure comprises an antibody light chain constant (CL) domain. In some embodiments, the CL domain is a kappa light chain constant domain (C_{K}), e.g., a human kappa light chain constant domain. In some embodiments, the CL domain is a lambda light chain constant domain (C_{L}), e.g., a human lambda light chain constant domain.

### Target Antigens

The multispecific antibodies of the present disclosure provide a framework/platform/configuration in which a variety of antigen binding sites and variable domains targeting a variety of potential target antigens are contemplated for use. In some embodiments, the first and second antigens are different. In some embodiments, the first and second antigens represent different epitopes (e.g., non-overlapping, partially non-overlapping, or non-competing epitopes) of the same antigen or target, e.g., target polypeptide. In some embodiments, a multispecific antibody of the present disclosure binds to two, three, or four different antigens. In some embodiments, a multispecific antibody of the present disclosure binds to two, three, or four different epitopes of the same antigen or target, e.g., target polypeptide. In some embodiments, a multispecific antibody of the present disclosure comprises two, three, or four different antigen binding sites that specifically bind the same antigen or target, e.g., target polypeptide. In some embodiments, the different antigen binding sites specifically bind two, three, or four different epitopes of the same antigen or target, e.g., target polypeptide.

As is known in the art, variable domains of the heavy chain and light chain (VH and VL, respectively) of an antibody generally have similar structures, with each domain comprising four conserved framework regions (FRs) and three complementarity-determining regions (CDRs). (See, e.g., Kindt et al. Kuby Immunology, 6^{th} ed., W. H. Freeman and Co., page 91 (2007).) Framework (or “FR” as used herein) can refer to variable domain residues other than the CDR residues. The FR of a variable domain generally consists of four FR domains: FR1, FR2, FR3, and FR4. Accordingly, the CDR and FR sequences generally appear in the following sequence in VH (or VL): FR1-CDR1-FR2-CDR2-FR3-CDR3-FR4. In some embodiments, a FR1, FR2, FR3, and/or FR4 of the present disclosure refers to a human framework region, i.e., of the VH or VL domain.

In some embodiments, the multispecific antibody comprises one or more antigen binding sites that binds to human Dectin-1. In some embodiments, the multispecific antibody comprises one or more antigen binding sites that binds to human Dectin-1 expressed on the surface of a macrophage, monocyte, dendritic cell, or granulocyte. In some embodiments, the multispecific antibody comprises one or more antigen binding sites that binds to human Dectin-1 isoform A and/or human Dectin-1 isoform B. In some embodiments, human Dectin-1 isoform A comprises the amino acid sequence MEYHPDLENLDEDGYTQLHFDSQSNTRIAVVSEKGSCAASPPWRLIAVILGILCLVILVIAVV LGTMAIWRSNSGSNTLENGYFLSRNKENHSQPTQSSLEDSVTPTKAVKTTGVLSSPCPPNWII YEKSCYLFSMSLNSWDGSKRQCWQLGSNLLKIDSSNELGFIVKQVSSQPDNSFWIGLSRPQT EVPWLWEDGSTFSSNLFQIRTTATQENPSPNCVWIHVSVIYDQLCSVPSYSICEKKFSM (SEQ ID NO: 13). In some embodiments, human Dectin-1 isoform B comprises the amino acid sequence MEYHPDLENLDEDGYTQLHFDSQSNTRIAVVSEKGSCAASPPWRLIAVILGILCLVILVIAVV LGTMGVLSSPCPPNWIIYEKSCYLFSMSLNSWDGSKRQCWQLGSNLLKIDSSNELGFIVKQV SSQPDNSFWIGLSRPQTEVPWLWEDGSTESSNLFQIRTTATQENPSPNCVWIHVSVIYDQLCS VPSYSICEKKFSM (SEQ ID NO:14).

In some embodiments, the multispecific antibody or antigen binding site thereof binds to human Dectin-1 expressed on the surface of a cell with an EC50 of less than 5 nM, less than 2 nM, less than 1 nM, or less than 0.5 nM. In some embodiments, the multispecific antibody or antigen binding site thereof is capable of binding to human Dectin-1 and monkey Dectin-1, e.g., cynomolgus Dectin-1.

In some embodiments, a multispecific antibody comprises one or more anti-Dectin-1 antigen binding sites from antibody 2M24, which is described in International Appl. No. PCT/US2021/071752, filed Oct. 6, 2021, or variant(s) thereof.

Multiple definitions for the CDR sequences of antibody variable domains are known in the art; see, e.g., Kabat (Sequences of Proteins of Immunological Interest, Fifth Edition, NIH Publication 91-3242, Bethesda MD (1991), vols. 1-3) and Chothia. Unless otherwise specified, CDR sequences are described herein according to the definition of IMGT. See, e.g., www.imgt.org/IMGTScientificChart/Nomenclature/IMGT-FRCDRdefinition.html.

In some embodiments, the antigen binding site that binds to Dectin-1 comprises a VH domain comprising a CDR-H1 comprising the amino acid sequence GYTFTDYY (SEQ ID NO:15), a CDR-H2 comprising the amino acid sequence INPNSGDT (SEQ ID NO:16), and a CDR-H3 comprising the amino acid sequence ARNSGSYSFGY (SEQ ID NO:17) and/or a VL domain comprising a CDR-L1 comprising the amino acid sequence QGISSW (SEQ ID NO:18), a CDR-L2 comprising the amino acid sequence GAS, and a CDR-L3 comprising the amino acid sequence QQAYSFPFT (SEQ ID NO:20). In some embodiments, the antigen binding site that binds to Dectin-1 comprises a VH domain comprising a CDR-H1 comprising the amino acid sequence GYTFTDYY (SEQ ID NO:15), a CDR-H2 comprising the amino acid sequence INPNSGDT (SEQ ID NO: 16), and a CDR-H3 comprising the amino acid sequence ARNSGSYSFGY (SEQ ID NO:17) and a VL domain comprising a CDR-L1 comprising the amino acid sequence QGISSW (SEQ ID NO: 18), a CDR-L2 comprising the amino acid sequence GAS, and a CDR-L3 comprising the amino acid sequence QQAYSFPFT (SEQ ID NO:20).

In some embodiments, the antigen binding site that binds to Dectin-1 comprises a VH domain comprising a CDR-H1 comprising the amino acid sequence DYYI (SEQ ID NO:21), a CDR-H2 comprising the amino acid sequence WINPNSGDTNYAQKFQG (SEQ ID NO:22), and a CDR-H3 comprising the amino acid sequence NSGSYSFGY (SEQ ID NO:23) and/or a VL domain comprising a CDR-L1 comprising the amino acid sequence RASQGISSWLA (SEQ ID NO:24), a CDR-L2 comprising the amino acid sequence GASSLQS (SEQ ID NO:25), and a CDR-L3 comprising the amino acid sequence QQAYSFPFT (SEQ ID NO:26). In some embodiments, the antigen binding site that binds to Dectin-1 comprises a VH domain comprising a CDR-H1 comprising the amino acid sequence DYYI (SEQ ID NO:21), a CDR-H2 comprising the amino acid sequence WINPNSGDTNYAQKFQG (SEQ ID NO:22), and a CDR-H3 comprising the amino acid sequence NSGSYSFGY (SEQ ID NO:23) and a VL domain comprising a CDR-L1 comprising the amino acid sequence RASQGISSWLA (SEQ ID NO:24), a CDR-L2 comprising the amino acid sequence GASSLQS (SEQ ID NO:25), and a CDR-L3 comprising the amino acid sequence QQAYSFPFT (SEQ ID NO:26).

In some embodiments, the antigen binding site that binds to Dectin-1 comprises one, two, or three CDR sequences from a VH domain comprising the amino acid sequence QVQLVQSGAEVKKPGASVKVSCKSSGYTFTDYYIHWVRQAPGQGLEWMGWINPNSGDTN YAQKFQGRITMTRDTSISTAYLELSRLRSDDTAVFYCARNSGSYSFGYWGQGTLVTVSS (SEQ ID NO:27) and/or one, two, or three CDR sequences from a VL domain comprising the amino acid sequence DIQMTQSPSSVSASVGDRVTITCRASQGISSWLAWYQQKPGKAPKLLIFGASSLQSGVPSRFS GSGSGTDFTLTVSSLQPEDFATYYCQQAYSFPFTFGPGTKVDIE (SEQ ID NO:28).

In some embodiments, the antigen binding site that binds to Dectin-1 comprises a VH domain comprising the amino acid sequence QVQLVQSGAEVKKPGASVKVSCKSSGYTFTDYYIHWVRQAPGQGLEWMGWINPNSGDTN YAQKFQGRITMTRDTSISTAYLELSRLRSDDTAVFYCARNSGSYSFGYWGQGTLVTVSS (SEQ ID NO:27) and/or a VL domain comprising the amino acid sequence DIQMTQSPSSVSASVGDRVTITCRASQGISSWLAWYQQKPGKAPKLLIFGASSLQSGVPSRFS GSGSGTDFTLTVSSLQPEDFATYYCQQAYSFPFTFGPGTKVDIE (SEQ ID NO:28). In some embodiments, the antigen binding site that binds to Dectin-1 comprises a VH domain comprising the amino acid sequence QVQLVQSGAEVKKPGASVKVSCKSSGYTFTDYYIHWVRQAPGQGLEWMGWINPNSGDTN YAQKFQGRITMTRDTSISTAYLELSRLRSDDTAVFYCARNSGSYSFGYWGQGTLVTVSS (SEQ ID NO:27) and a VL domain comprising the amino acid sequence DIQMTQSPSSVSASVGDRVTITCRASQGISSWLAWYQQKPGKAPKLLIFGASSLQSGVPSRFS GSGSGTDFTLTVSSLQPEDFATYYCQQAYSFPFTFGPGTKVDIE (SEQ ID NO:28).

In some embodiments, a multispecific antibody of the present disclosure comprises a first polypeptide that comprises the amino acid sequence of QVQLVQSGAEVKKPGASVKVSCKSSGYTFTDYYIHWVRQAPGQGLEWMGWINPNSGDTN YAQKFQGRITMTRDTSISTAYLELSRLRSDDTAVFYCARNSGSYSFGYWGQGTLVTVSSAST KGVALHRPDVYLLPPAREQLNLRESATISCAVTGFSPADVFVQWMQRGQPLSPEKYVTSAP MPEPQAPGRYFAVSILTVSEEEWNTGETYTCVVAHEALPNRVTERTVDKSCDKTHTCPPCP APELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYTLP PSREEMTKNQVSLWCLVKGFYPSDIAVEWESNGQPENNYKTTPPVLDSDGSFFLYSKLTVD KSRWQQGNVFSCSVMHEALHNHYTQKSLSLSPG (SEQ ID NO:31), and a second polypeptide that comprises the amino acid sequence of DIQMTQSPSSVSASVGDRVTITCRASQGISSWLAWYQQKPGKAPKLLIFGASSLQSGVPSRES GSGSGTDFTLTVSSLQPEDFATYYCQQAYSFPFTFGPGTKVDIERTVALHRPDVYLLPPARE QLNLRESATIWCLVTGFSPADVFVQWMQRGQPLSPEKYVTSAPMPEPQAPGRYFAHSILTV SEEEWNTGETYTCVVAHEALPNRVTERTVDKSTGEC (SEQ ID NO:32). In some embodiments, a multispecific antibody of the present disclosure comprises a first polypeptide that comprises the amino acid sequence of QVQLVQSGAEVKKPGASVKVSCKSSGYTFTDYYIHWVRQAPGQGLEWMGWINPNSGDTN YAQKFQGRITMTRDTSISTAYLELSRLRSDDTAVFYCARNSGSYSFGYWGQGTLVTVSSAST KGVALHRPDVYLLPPAREQLNLRESATISCAVTGFSPADVFVQWMQRGQPLSPEKYVTSAP MPEPQAPGRYFAVSILTVSEEEWNTGETYTCVVAHEALPNRVTERTVDKSCDKTHTCPPCP APELLGGPSVFLFPPKPKDTLMISRTPEVTCVVVDVSHEDPEVKFNWYVDGVEVHNAKTKP REEQYNSTYRVVSVLTVLHQDWLNGKEYKCKVSNKALPAPIEKTISKAKGQPREPQVYTLP PSREEMTKNQVSLWCLVKGFYPSDIAVEWESNGQPENNYKTTPPVLDSDGSFFLYSKLTVD KSRWQQGNVFSCSVMHEALHNHYTQKSLSLSPGK (SEQ ID NO:33), and a second polypeptide that comprises the amino acid sequence of DIQMTQSPSSVSASVGDRVTITCRASQGISSWLAWYQQKPGKAPKLLIFGASSLQSGVPSRFS GSGSGTDFTLTVSSLQPEDFATYYCQQAYSFPFTFGPGTKVDIERTVALHRPDVYLLPPARE QLNLRESATIWCLVTGFSPADVFVQWMQRGQPLSPEKYVTSAPMPEPQAPGRYFAHSILTV SEEEWNTGETYTCVVAHEALPNRVTERTVDKSTGEC (SEQ ID NO:32). In some embodiments, a multispecific antibody of the present disclosure comprises a first polypeptide that comprises the amino acid sequence of SEQ ID NO:31 or SEQ ID NO:33, a second polypeptide that comprises the amino acid sequence of SEQ ID NO:32, a third polypeptide that comprises the amino acid sequence of SEQ ID NO:34 or 35, and a fourth polypeptide that comprises the amino acid sequence of SEQ ID NO:36.

As is known in the art, the C-terminal lysine of some antibody heavy chain polypeptides may be cleaved off in some fraction of molecules. Therefore, in some embodiments, an arm or multispecific binding molecule of the present disclosure may be present (e.g., in a composition) comprising a mixture of species in which some polypeptides retain the C-terminal lysine and some do not.

In some embodiments, the antigen binding site that binds to Dectin-1 comprises one, two, or three CDR-H1, CDR-H2, and/or CDR-H3 sequences from a single VH domain of an anti-Dectin-1 antibody shown in Table 1. In some embodiments, the antigen binding site that binds to Dectin-1 comprises one, two, or three CDR-L1, CDR-L2, and/or CDR-L3 sequences from a single VL domain of an anti-Dectin-1 antibody shown in Table 1. In some embodiments, the antigen binding site that binds to Dectin-1 comprises CDR-H1, CDR-H2, and CDR-H3 sequences from a single VH domain of an anti-Dectin-1 antibody shown in Table 1. In some embodiments, the antigen binding site that binds to Dectin-1 comprises CDR-L1, CDR-L2, and CDR-L3 sequences from a single VL domain of an anti-Dectin-1 antibody shown in Table 1. In some embodiments, the antigen binding site that binds to Dectin-1 comprises CDR-H1, CDR-H2, and CDR-H3 sequences from a single VH domain of an anti-Dectin-1 antibody shown in Table 1 and CDR-L1, CDR-L2, and CDR-L3 sequences from a single VL domain of an anti-Dectin-1 antibody shown in Table 1. Any of the sets of heavy chain CDRs of a single VH domain from Table 1 can be combined with any of the sets of light chain CDRs of a single VL domain from Table 2 in an antigen binding site of the present disclosure.

TABLE 1
Exemplary anti-Dectin-1 antibody CDR sequences.

| Name | CDR-H1 | CDR-H2 | CDR-H3 | CDR-LI | CDR-L2 | CDR-L3 |
|---|---|---|---|---|---|---|
| 2M24 | DYYI | WINPNSG | NSGSYSFG | RASQGIS | GASSLQS | QQAYSFPFT |
| (Kabat) | (SEQ ID | DTNYAQ | Y (SEQ ID | SWLA | (SEQ ID NO: | (SEQ ID NO: |
| | NO: 21) | KFQG | NO: 23) | (SEQ ID | 25) | 26) |
| | | (SEQ ID | | NO: 24) | | |
| | | NO: 22) | | | | |
| 2M24 | GYTFTDY | INPNSGD | ARNSGSYS | QGISSW | GAS | QQAYSFPFT |
| (IMGT) | Y (SEQ ID | T (SEQ ID | FGY (SEQ | (SEQ ID | | (SEQ ID NO: |
| | NO: 15) | NO: 16) | ID NO: 17) | NO: 18) | | 20) |
| 2M24 | GYTFTDY | NPNSGD | NSGSYSFG | RASQGIS | GASSLQS | QQAYSFPFT |
| (Chothia) | (SEQ ID | (SEQ ID | Y (SEQ ID | SWLA | (SEQ ID NO: | (SEQ ID NO: |
| | NO: 46) | NO: 47) | NO: 23) | (SEQ ID | 25) | 26) |
| | | | | NO: 24) | | |
| 2M24A.1. | AYYI | WINPNSG | NSGSYSFG | | | |
| 11 | (SEQ ID | DTNYAQ | Y (SEQ ID | | | |
| (Kabat) | NO: 48) | KFQG | NO: 23) | | | |
| | | (SEQ ID | | | | |
| | | NO: 22) | | | | |
| 2M24A.1. | GYTFTAY | INPNSGD | ARNSGSYS | | | |
| 11 | Y (SEQ | T (SEQ ID | FGY (SEQ | | | |
| (IMGT) | ID NO: 49) | NO: 16) | ID NO: 17) | | | |
| 2M24A.1. | GYTFTAY | NPNSGD | NSGSYSFG | | | |
| 11 | (SEQ ID | (SEQ ID | Y (SEQ ID | | | |
| (Chothia) | NO: 50) | NO: 47) | NO: 23) | | | |
| 2M24A.1. | DYYI | WINPNSG | NSGSYSFG | | | |
| 12 | (SEQ ID | ATNYAQ | Y (SEQ ID | | | |
| (Kabat) | NO: 21) | KFQG | NO: 23) | | | |
| | | (SEQ ID | | | | |
| | | NO: 51) | | | | |
| 2M24A.1. | GYTFTDY | INPNSGA | ARNSGSYS | | | |
| 12 | Y (SEQ | T (SEQ ID | FGY (SEQ | | | |
| (IMGT) | ID NO: 15) | NO: 52) | ID NO: 17) | | | |
| 2M24A.1. | GYTFTDY | NPNSGA | NSGSYSFG | | | |
| 12 | (SEQ ID | (SEQ ID | Y (SEQ ID | | | |
| (Chothia) | NO: 46) | NO: 53) | NO: 23) | | | |
| 2M24A.1. | DYYI | WINPNSG | ASGSYSFG | | | |
| (Kabat) | (SEQ ID | DTNYAQ | Y (SEQ ID | | | |
| | NO: 21) | KFQG | NO: 54) | | | |
| | | (SEQ ID | | | | |
| | | NO: 22) | | | | |
| 2M24A.1. | GYTFTDY | INPNSGD | ARASGSYS | | | |
| 13 | Y (SEQ ID | T (SEQ ID | FGY (SEQ | | | |
| (IMGT) | NO: 15) | NO: 16) | ID NO: 55) | | | |
| 2M24A.1. | GYTFTDY | NPNSGD | ASGSYSFG | | | |
| 13 | (SEQ ID | (SEQ ID | Y (SEQ ID | | | |
| (Chothia) | NO: 46) | NO: 47) | NO: 54) | | | |
| 2M24A.1. | DYYI | WINPNSG | NSGSASFG | | | |
| 14 | (SEQ ID | DTNYAQ | Y (SEQ ID | | | |
| (Kabat) | NO: 21) | KFQG | NO: 56) | | | |
| | | (SEQ ID | | | | |
| | | NO: 22) | | | | |
| 2M24A.1. | GYTFTDY | INPNSGD | ARNSGSAS | | | |
| 14 | Y (SEQ | T (SEQ ID | FGY (SEQ | | | |
| (IMGT) | ID NO: 15) | NO: 16) | ID NO: 57) | | | |
| 2M24A.1. | GYTFTDY | NPNSGD | NSGSASFG | | | |
| 14 | (SEQ ID | (SEQ ID | Y (SEQ ID | | | |
| (Chothia) | NO: 46) | NO: 47) | NO: 56) | | | |
| 2M24A.1. | DYYI | WINPNSG | NSGSYSFG | | | |
| 15 | (SEQ ID | DTNYAQ | Y (SEQ ID | | | |
| (Kabat) | NO: 21) | KFQG | NO: 23) | | | |
| | | (SEQ ID | | | | |
| | | NO: 22) | | | | |
| 2M24A.1. | GYTFTDY | INPNSGD | AANSGSYS | | | |
| 15 | Y (SEQ ID | T (SEQ ID | FGY (SEQ | | | |
| (IMGT) | NO: 15) | NO: 16) | ID NO: 58) | | | |
| 2M24A.1. | GYTFTDY | NPNSGD | NSGSYSFG | | | |
| 15 | (SEQ ID | (SEQ ID | Y (SEQ ID | | | |
| (Chothia) | NO: 46) | NO: 47) | NO: 23) | | | |
| 2M24A.1. | DYYI | WINPNSG | NAGSYSFG | | | |
| 16 | (SEQ ID | DTNYAQ | Y (SEQ ID | | | |
| (Kabat) | NO: 21) | KFQG | NO: 59) | | | |
| | | (SEQ ID | | | | |
| | | NO: 22) | | | | |
| 2M24A.1. | GYTFTDY | INPNSGD | ARNAGSYS | | | |
| 16 | Y (SEQ ID | T (SEQ ID | FGY (SEQ | | | |
| (IMGT) | NO: 15) | NO: 16) | ID NO: 60) | | | |
| 2M24A.1. | GYTFTDY | NPNSGD | NAGSYSFG | | | |
| 16 | (SEQ ID | (SEQ ID | Y (SEQ ID | | | |
| (Chothia) | NO: 46) | NO: 47) | NO: 59) | | | |
| 2M24A.1. | DYYI | WINPNSG | NSASYSFG | | | |
| 17 | (SEQ ID | DTNYAQ | Y (SEQ ID | | | |
| (Kabat) | NO: 21) | KFQG | NO: 61) | | | |
| | | (SEQ ID | | | | |
| | | NO: 22) | | | | |
| 2M24A.1. | GYTFTDY | INPNSGD | ARNSASYS | | | |
| 17 | Y (SEQ ID | T (SEQ ID | FGY (SEQ | | | |
| (IMGT) | NO: 15) | NO: 16) | ID NO: 62) | | | |
| 2M24A.1. | GYTFTDY | NPNSGD | NSASYSFG | | | |
| 17 | (SEQ ID | (SEQ ID | Y (SEQ ID | | | |
| (Chothia) | NO: 46) | NO: 47) | NO: 61) | | | |
| 2M24A.1. | DYYI | WINPNSG | NSGAYSFG | | | |
| 18 | (SEQ ID | DTNYAQ | Y (SEQ ID | | | |
| (Kabat) | NO: 21) | KFQG | NO: 63) | | | |
| | | (SEQ ID | | | | |
| | | NO: 22) | | | | |
| 2M24A.1. | GYTFTDY | INPNSGD | ARNSGAYS | | | |
| 18 | Y (SEQ ID | T (SEQ ID | FGY (SEQ | | | |
| (IMGT) | NO: 15) | NO: 16) | ID NO: 64) | | | |
| 2M24A.1. | GYTFTDY | NPNSGD | NSGAYSFG | | | |
| 18 | (SEQ ID | (SEQ ID | Y (SEQ ID | | | |
| (Chothia) | NO: 46) | NO: 47) | NO: 63) | | | |
| 2M24A.1. | DYYI | WINPNSG | NSGSYAFG | | | |
| 19 | (SEQ ID | DTNYAQ | Y (SEQ ID | | | |
| (Kabat) | NO: 21) | KFQG | NO: 79) | | | |
| | | (SEQ ID | | | | |
| | | NO: 22) | | | | |
| 2M24A.1. | GYTFTDY | INPNSGD | ARNSGSYA | | | |
| 19 | Y (SEQ ID | T (SEQ ID | FGY (SEQ | | | |
| (IMGT) | NO: 15) | NO: 16) | ID NO: 65) | | | |
| 2M24A.1. | GYTFTDY | NPNSGD | NSGSYAFG | | | |
| 19 | (SEQ ID | (SEQ ID | Y (SEQ ID | | | |
| (Chothia) | NO: 46) | NO: 47) | NO: 79) | | | |
| 2M24A.1. | DYYI | WINPNSG | NSGSYSAG | | | |
| 20 | (SEQ ID | DTNYAQ | Y (SEQ ID | | | |
| (Kabat) | NO: 21) | KFQG | NO: 66) | | | |
| | | (SEQ ID | | | | |
| | | NO: 22) | | | | |
| 2M24A.1. | GYTFTDY | INPNSGD | ARNSGSYS | | | |
| 20 | Y (SEQ ID | T (SEQ ID | AGY (SEQ | | | |
| (IMGT) | NO: 15) | NO: 16) | ID NO: 67) | | | |
| 2M24A.1. | GYTFTDY | NPNSGD | NSGSYSAG | | | |
| 20 | (SEQ ID | (SEQ ID | Y (SEQ ID | | | |
| (Chothia) | NO: 46) | NO: 47) | NO: 66) | | | |
| 2M24A.1. | DYYI | WINPNSG | NSGSYSFA | | | |
| 21 | (SEQ ID | DTNYAQ | Y (SEQ ID | | | |
| (Kabat) | NO: 21) | KFQG | NO: 68) | | | |
| | | (SEQ ID | | | | |
| | | NO: 22) | | | | |
| 2M24A.1. | GYTFTDY | INPNSGD | ARNSGSYS | | | |
| 21 | Y (SEQ ID | T (SEQ ID | FAY (SEQ | | | |
| (IMGT) | NO: 15) | NO: 16) | ID NO: 69) | | | |
| 2M24A.1. | GYTFTDY | NPNSGD | NSGSYSFA | | | |
| 21 | (SEQ ID | (SEQ ID | Y (SEQ ID | | | |
| (Chothia) | NO: 46) | NO: 47) | NO: 68) | | | |
| 2M24A.1. | DYYI | WINPNSG | NSGSYSFG | | | |
| 22 | (SEQ ID | DTNYAQ | A (SEQ ID | | | |
| (Kabat) | NO: 21) | KFQG | NO: 70) | | | |
| | | (SEQ ID | | | | |
| | | NO: 22) | | | | |
| 2M24A.1. | GYTFTDY | INPNSGD | ARNSGSYS | | | |
| 22 | Y (SEQ ID | T (SEQ ID | FGA (SEQ | | | |
| (IMGT) | NO: 15) | NO: 16) | ID NO: 71) | | | |
| 2M24A.1. | GYTFTDY | NPNSGD | NSGSYSFG | | | |
| 22 | (SEQ ID | (SEQ ID | A (SEQ ID | | | |
| (Chothia) | NO: 46) | NO: 47) | NO: 70) | | | |
| 2M24A.1. | | | | RASQGIS | GASSLQS | QQAASFPFT |
| 5 | | | | SWLA | (SEQ ID | (SEQ ID |
| (Kabat) | | | | (SEQ ID | NO: 25) | NO: 72) |
| | | | | NO: 24) | | |
| 2M24A.1. | | | | QGISSW | GAS | QQAASFPFT |
| 5 | | | | (SEQ ID | | (SEQ ID |
| (IMGT) | | | | NO: 18) | | NO: 72) |
| 2M24A.1. | | | | RASQGIS | GASSLQS | QQAASFPFT |
| 5 | | | | SWLA | (SEQ ID | (SEQ ID |
| (Chothia) | | | | (SEQ ID | NO: 25) | NO: 72) |
| | | | | NO: 24) | | |
| 2M24A.1. | | | | RASQGIS | GASSLQS | QQAFSFPFT |
| 10 | | | | SWLA | (SEQ ID | (SEQ ID |
| (Kabat) | | | | (SEQ ID | NO: 25) | NO: 73) |
| | | | | NO: 24) | | |
| 2M24A.1. | | | | QGISSW | GAS | QQAFSFPFT |
| 10 | | | | (SEQ ID | | (SEQ ID |
| (IMGT) | | | | NO: 18) | | NO: 73) |
| 2M24A.1. | | | | RASQGIS | GASSLQS | QQAFSFPFT |
| 10 | | | | SWLA | (SEQ ID | (SEQ ID |
| (Chothia) | | | | (SEQ ID | NO: 25) | NO: 73) |
| | | | | NO: 24) | | |
| 2M24A.1. | | | | RASQGIS | GASSLQS | AQAYSFPFT |
| 23 | | | | SWLA | (SEQ ID | (SEQ ID |
| (Kabat) | | | | (SEQ ID | NO: 25) | NO: 74) |
| | | | | NO: 24) | | |
| 2M24A.1. | | | | QGISSW | GAS | AQAYSFPFT |
| 23 | | | | (SEQ ID | | (SEQ ID |
| (IMGT) | | | | NO: 18) | | NO: 74) |
| 2M24A.1. | | | | RASQGIS | GASSLQS | AQAYSFPFT |
| 23 | | | | SWLA | (SEQ ID | (SEQ ID |
| (Chothia) | | | | (SEQ ID | NO: 25) | NO: 74) |
| | | | | NO: 24) | | |
| 2M24A.1. | | | | RASQGIS | GASSLQS | QAAYSFPFT |
| 24 | | | | SWLA | (SEQ ID | (SEQ ID |
| (Kabat) | | | | (SEQ ID | NO: 25) | NO: 75) |
| | | | | NO: 24) | | |
| 2M24A.1. | | | | QGISSW | GAS | QAAYSFPFT |
| 24 | | | | (SEQ ID | | (SEQ ID |
| (IMGT) | | | | NO: 18) | | NO: 75) |
| 2M24A.1. | | | | RASQGIS | GASSLQS | QAAYSFPFT |
| 24 | | | | SWLA | (SEQ ID | (SEQ ID |
| (Chothia) | | | | (SEQ ID | NO: 25) | NO: 75) |
| | | | | NO: 24) | | |
| 2M24A.1. | | | | RASQGIS | GASSLQS | QQAYAFPFT |
| 25 | | | | SWLA | (SEQ ID | (SEQ ID |
| (Kabat) | | | | (SEQ ID | NO: 25) | NO: 76) |
| | | | | NO: 24) | | |
| 2M24A.1. | | | | QGISSW | GAS | QQAYAFPFT |
| 25 | | | | (SEQ ID | | (SEQ ID |
| (IMGT) | | | | NO: 18) | | NO: 76) |
| 2M24A.1. | | | | RASQGIS | GASSLQS | QQAYAFPFT |
| 25 | | | | SWLA | (SEQ ID | (SEQ ID |
| (Chothia) | | | | (SEQ ID | NO: 25) | NO: 76) |
| | | | | NO: 24) | | |
| 2M24A.1. | | | | RASQGIS | GASSLQS | QQAYSAPFT |
| 26 | | | | SWLA | (SEQ ID | (SEQ ID NO: |
| (Kabat) | | | | (SEQ ID | NO: 25) | 77) |
| | | | | NO: 24) | | |
| 2M24A.1. | | | | QGISSW | GAS | QQAYSAPFT |
| 26 | | | | (SEQ ID | | (SEQ ID NO: |
| (IMGT) | | | | NO: 18) | | 77) |
| 2M24A.1. | | | | RASQGIS | GASSLQS | QQAYSAPFT |
| 26 | | | | SWLA | (SEQ ID | (SEQ ID NO: |
| (Chothia) | | | | (SEQ ID | NO: 25) | 77) |
| | | | | NO: 24) | | |
| 2M24A.1. | | | | RASQGIS | GASSLQS | QQAYSFAFT |
| 27 | | | | SWLA | (SEQ ID | (SEQ ID |
| (Kabat) | | | | (SEQ ID | NO: 25) | NO: 78) |
| | | | | NO: 24) | | |
| | | | | (SEQ ID | | |
| | | | | NO: 24) | | |
| 2M24A.1. | | | | QGISSW | GAS | QQAYSFAFT |
| 27 | | | | (SEQ ID | | (SEQ ID |
| (IMGT) | | | | NO: 18) | | NO: 78) |
| 2M24A.1. | | | | RASQGIS | GASSLQS | QQAYSFAFT |
| 27 | | | | SWLA | (SEQ ID | (SEQ ID |
| (Chothia) | | | | (SEQ ID | NO: 25) | NO: 78) |
| | | | | NO: 24) | | |
| 2M24A.1. | | | | RASQGIS | GASSLQS | QQAYSFPAT |
| 28 | | | | SWLA | (SEQ ID | (SEQ ID |
| (Kabat) | | | | (SEQ ID | NO: 25) | NO: 80) |
| | | | | NO: 24) | | |
| 2M24A.1. | | | | QGISSW | GAS | QQAYSFPAT |
| 28 | | | | (SEQ ID | | (SEQ ID |
| (IMGT) | | | | NO: 18) | | NO: 80) |
| 2M24A.1. | | | | RASQGIS | GASSLQS | QQAYSFPAT |
| 28 | | | | SWLA | (SEQ ID | (SEQ ID |
| (Chothia) | | | | (SEQ ID | NO: 25) | NO: 80) |
| | | | | NO: 24) | | |
| 2M24A.1. | | | | RASQGIS | GASSLQS | QQAYSFPFA |
| 29 | | | | SWLA | (SEQ ID | (SEQ ID |
| (Kabat) | | | | (SEQ ID | NO: 25) | NO: 81) |
| | | | | NO: 24) | | |
| 2M24A.1. | | | | QGISSW | GAS | QQAYSFPFA |
| 29 | | | | (SEQ ID | | (SEQ ID |
| (IMGT) | | | | NO: 18) | | NO: 81) |
| 2M24A.1. | | | | RASQGIS | GASSLQS | QQAYSFPFA |
| 29 | | | | SWLA | (SEQ ID | (SEQ ID |
| (Chothia) | | | | (SEQ ID | NO: 25) | NO: 81) |
| | | | | NO: 24) | | |

In some embodiments, the antigen binding site that binds to Dectin-1 comprises one, two, or three CDR sequences from a VH domain shown in Table 2 and/or one, two, or three CDR sequences from a VL domain shown in Table 2. In some embodiments, the antigen binding site that binds to Dectin-1 comprises a VH domain sequence shown in Table 2 and/or a VL domain sequence shown in Table 2. In some embodiments, the antigen binding site that binds to Dectin-1 comprises a VH domain sequence shown in Table 2 and a VL domain sequence shown in Table 2. Any of the VH domain sequences of Table 2 can be combined with any of the VL domain sequences of Table 2 in an antigen binding site of the present disclosure.

TABLE 2
Exemplary anti-Dectin-1 antibody variable domain sequences

| Name | VH | VL |
|---|---|---|
| 2M24 | QVQLVQSGAEVKKPGASVKVSCKSSG | DIQMTQSPSSVSASVGDRVTITCR |
| | YTFTDYYIHWVRQAPGQGLEWMGWI | ASQGISSWLAWYQQKPGKAPKLL |
| | NPNSGDTNYAQKFQGRITMTRDTSIST | IFGASSLQSGVPSRFSGSGSGTDFT |
| | AYLELSRLRSDDTAVFYCARNSGSYSF | LTVSSLQPEDFATYYCQQAYSFPF |
| | GYWGQGTLVTVSS (SEQ ID NO: 27) | TFGPGTKVDIE (SEQ ID NO: 28) |
| 2M24 | QVQLVQSGAEVKKPGASVKVSCKSSGYTF | co |
| A.1.11 | TAYYIHWVRQAPGQGLEWMGWINPNSGD | |
| | TNYAQKFQGRITMTRDTSISTAYLELSRLR | |
| | SDDTAVFYCARNSGSYSFGYWGQGTLVT | |
| | VSS (SEQ ID NO: 82) | |
| 2M24 | QVQLVQSGAEVKKPGASVKVSCKSSGYTF | |
| A.1.12 | TDYYIHWVRQAPGQGLEWMGWINPNSGA | |
| | TNYAQKFQGRITMTRDTSISTAYLELSRLR | |
| | SDDTAVFYCARNSGSYSFGYWGQGTLVT | |
| | VSS (SEQ ID NO: 83) | |
| 2M24 | QVQLVQSGAEVKKPGASVKVSCKSSGYTF | |
| A.1.13 | TDYYIHWVRQAPGQGLEWMGWINPNSGD | |
| | TNYAQKFQGRITMTRDTSISTAYLELSRLR | |
| | SDDTAVFYCARASGSYSFGYWGQGTLVT | |
| | VSS (SEQ ID NO: 84) | |
| 2M24 | QVQLVQSGAEVKKPGASVKVSCKSSGYTF | |
| A.1.14 | TDYYIHWVRQAPGQGLEWMGWINPNSGD | |
| | TNYAQKFQGRITMTRDTSISTAYLELSRLR | |
| | SDDTAVFYCARNSGSASFGYWGQGTLVT | |
| | VSS (SEQ ID NO: 85) | |
| 2M24 | QVQLVQSGAEVKKPGASVKVSCKSSGYTF | |
| A.1.15 | TDYYIHWVRQAPGQGLEWMGWINPNSGD | |
| | TNYAQKFQGRITMTRDTSISTAYLELSRLR | |
| | SDDTAVFYCAANSGSYSFGYWGQGTLVT | |
| | VSS (SEQ ID NO: 86) | |
| 2M24 | QVQLVQSGAEVKKPGASVKVSCKSSGYTF | |
| A.1.16 | TDYYIHWVRQAPGQGLEWMGWINPNSGD | |
| | TNYAQKFQGRITMTRDTSISTAYLELSRLR | |
| | SDDTAVFYCARNAGSYSFGYWGQGTLVT | |
| | VSS (SEQ ID NO: 87) | |
| 2M24 | QVQLVQSGAEVKKPGASVKVSCKSSGYTF | |
| A.1.17 | TDYYIHWVRQAPGQGLEWMGWINPNSGD | |
| | TNYAQKFQGRITMTRDTSISTAYLELSRLR | |
| | SDDTAVFYCARNSASYSFGYWGQGTLVT | |
| | VSS (SEQ ID NO: 88) | |
| 2M24 | QVQLVQSGAEVKKPGASVKVSCKSSGYTF | |
| A.1.18 | TDYYIHWVRQAPGQGLEWMGWINPNSGD | |
| | TNYAQKFQGRITMTRDTSISTAYLELSRLR | |
| | SDDTAVFYCARNSGAYSFGYWGQGTLVT | |
| | VSS (SEQ ID NO: 89) | |
| 2M24 | QVQLVQSGAEVKKPGASVKVSCKSSGYTF | |
| A.1.19 | TDYYIHWVRQAPGQGLEWMGWINPNSGD | |
| | TNYAQKFQGRITMTRDTSISTAYLELSRLR | |
| | SDDTAVFYCARNSGSYAFGYWGQGTLVT | |
| | VSS (SEQ ID NO: 90) | |
| 2M24 | QVQLVQSGAEVKKPGASVKVSCKSSGYTF | |
| A.1.20 | TDYYIHWVRQAPGQGLEWMGWINPNSGD | |
| | TNYAQKFQGRITMTRDTSISTAYLELSRLR | |
| | SDDTAVFYCARNSGSYSAGYWGQGTLVT | |
| | VSS (SEQ ID NO: 91) | |
| 2M24 | QVQLVQSGAEVKKPGASVKVSCKSSGYTF | |
| A.1.21 | TDYYIHWVRQAPGQGLEWMGWINPNSGD | |
| | TNYAQKFQGRITMTRDTSISTAYLELSRLR | |
| | SDDTAVFYCARNSGSYSFAYWGQGTLVT | |
| | VSS (SEQ ID NO: 92) | |
| 2M24 | QVQLVQSGAEVKKPGASVKVSCKSSGYTF | |
| A.1.22 | TDYYIHWVRQAPGQGLEWMGWINPNSGD | |
| | TNYAQKFQGRITMTRDTSISTAYLELSRLR | |
| | SDDTAVFYCARNSGSYSFGAWGQGTLVT | |
| | VSS (SEQ ID NO: 93) | |
| 2M24 | | DIQMTQSPSSVSASVGDRVTITCRAS |
| A.1.5 | | QGISSWLAWYQQKPGKAPKLLIFGA |
| | | SSLQSGVPSRFSGSGSGTDFTLTVSSL |
| | | QPEDFATYYCQQAASFPFTFGPGTKV |
| | | DIE (SEQ ID NO: 94) |
| 2M24 | | DIQMTQSPSSVSASVGDRVTITCRAS |
| A.1.10 | | QGISSWLAWYQQKPGKAPKLLIFGA |
| | | SSLQSGVPSRFSGSGSGTDFTLTVSSL |
| | | QPEDFATYYCQQAFSFPFTFGPGTKV |
| | | DIE (SEQ ID NO: 95) |
| 2M24 | | DIQMTQSPSSVSASVGDRVTITCRAS |
| A.1.23 | | QGISSWLAWYQQKPGKAPKLLIFGA |
| | | SSLQSGVPSRFSGSGSGTDFTLTVSSL |
| | | QPEDFATYYCAQAYSFPFTFGPGTKV |
| | | DIE (SEQ ID NO: 96) |
| 2M24 | | DIQMTQSPSSVSASVGDRVTITCRAS |
| A.1.24 | | QGISSWLAWYQQKPGKAPKLLIFGA |
| | | SSLQSGVPSRFSGSGSGTDFTLTVSSL |
| | | QPEDFATYYCQAAYSFPFTFGPGTKV |
| | | DIE (SEQ ID NO: 97) |
| 2M24 | | DIQMTQSPSSVSASVGDRVTITCRAS |
| A.1.25 | | QGISSWLAWYQQKPGKAPKLLIFGA |
| | | SSLQSGVPSRFSGSGSGTDFTLTVSSL |
| | | QPEDFATYYCQQAYAFPFTFGPGTK |
| | | VDIE (SEQ ID NO: 98) |
| 2M24 | | DIQMTQSPSSVSASVGDRVTITCRAS |
| A.1.26 | | QGISSWLAWYQQKPGKAPKLLIFGA |
| | | SSLQSGVPSRFSGSGSGTDFTLTVSSL |
| | | QPEDFATYYCQQAYSAPFTFGPGTK |
| | | VDIE (SEQ ID NO: 99) |
| 2M24 | | DIQMTQSPSSVSASVGDRVTITCRAS |
| A.1.27 | | QGISSWLAWYQQKPGKAPKLLIFGA |
| | | SSLQSGVPSRFSGSGSGTDFTLTVSSL |
| | | QPEDFATYYCQQAYSFAFTFGPGTK |
| | | VDIE (SEQ ID NO: 100) |
| 2M24 | | DIQMTQSPSSVSASVGDRVTITCRAS |
| A.1.28 | | QGISSWLAWYQQKPGKAPKLLIFGA |
| | | SSLQSGVPSRFSGSGSGTDFTLTVSSL |
| | | QPEDFATYYCQQAYSFPATFGPGTK |
| | | VDIE (SEQ ID NO: 101) |
| 2M24 | | DIQMTQSPSSVSASVGDRVTITCRAS |
| A.1.29 | | QGISSWLAWYQQKPGKAPKLLIFGA |
| | | SSLQSGVPSRFSGSGSGTDFTLTVSSL |
| | | QPEDFATYYCQQAYSFPFAFGPGTK |
| | | VDIE (SEQ ID NO: 102) |

In some embodiments, a multispecific antibody of the present disclosure comprises one or more antigen binding sites that bind to human Dectin-1 and one or more antigen binding sites that bind to a target of interest, e.g., other than human Dectin-1.

In some embodiments, a multispecific antibody of the present disclosure comprises one or more antigen binding sites that specifically bind a disease-causing agent or antigen thereof. In some embodiments, the disease-causing agent is a bacterial cell, fungal cell, virus, senescent cell, tumor cell, protein aggregate, LDL particle, mast cell, eosinophil, ILC2 cell, or inflammatory immune cell. In some embodiments, the target antigen is expressed on the surface of the bacterial cell, fungal cell, senescent cell, tumor cell, mast cell, eosinophil, ILC2 cell, or inflammatory immune cell. In some embodiments, the target antigen is from a virus. In some embodiments, the target antigen is a protein aggregate or monomer thereof, e.g., amyloid beta (such as in Alzheimer's disease), or lambda or kappa light chain amyloid (such as in light chain amyloidosis). In some embodiments, e.g., for oncology applications, the target antigen is CD70, HER2, DLL3, NECTIN-4, TROP-2, Mesothelin, LIV-1, C-MET, FOLR1, CD20, CCR8, CD33, or EGFR, e.g., as expressed on the surface of a cancer cell.

In some embodiments, the multispecific antibody comprises at least one antigen binding site that specifically binds an antigen expressed on a cell surface of an immune cell and at least one antigen binding site that specifically binds a disease-causing agent. In some embodiments, the multispecific antibody comprises at least one antigen binding site that specifically binds an antigen expressed on a cell surface of a myeloid cell and at least one antigen binding site that specifically binds a disease-causing agent.

In some embodiments, the target antigen is CD20, e.g., human CD20. In some embodiments, the antigen binding site that binds CD20 comprises a VH domain comprising a CDR-H1, CDR-H2, and CDR-H3 sequence from the VH domain sequence QVQLQQPGAELVKPGASVKMSCKASGYTFTSYNMHWVKQTPGRGLEWIGAIYPGNGDTS YNQKFKGKATLTADKSSSTAYMQLSSLTSEDSAVYYCARSTYYGGDWYFNVWGAGTTVT VSA (SEQ ID NO:29) and/or a VL domain comprising a CDR-L1, CDR-L2, and CDR-L3 sequence from the VL domain sequence QIVLSQSPAILSASPGEKVTMTCRASSSVSYIHWFQQKPGSSPKPWIYATSNLASGVPVRESG SGSGTSYSLTISRVEAEDAATYYCQQWTSNPPTFGGGTKLEIK (SEQ ID NO:30). In some embodiments, the antigen binding site that binds CD20 comprises a VH domain that comprises the sequence QVQLQQPGAELVKPGASVKMSCKASGYTFTSYNMHWVKQTPGRGLEWIGAIYPGNGDTS YNQKFKGKATLTADKSSSTAYMQLSSLTSEDSAVYYCARSTYYGGDWYFNVWGAGTTVT VSA (SEQ ID NO:29) and/or a VL domain that comprises the sequence QIVLSQSPAILSASPGEKVTMTCRASSSVSYIHWFQQKPGSSPKPWIYATSNLASGVPVRESG SGSGTSYSLTISRVEAEDAATYYCQQWTSNPPTFGGGTKLEIK (SEQ ID NO:30). In some embodiments, the antigen binding site that binds CD20 comprises the VH and VL domain sequences from rituximab. In some embodiments, the arm comprising the antigen binding site that specifically binds CD20 comprises an antibody heavy chain polypeptide comprising the amino acid sequence of QVQLQQPGAELVKPGASVKMSCKASGYTFTSYNMHWVKQTPGRGLEWIGAIYPGNGDTS YNQKFKGKATLTADKSSSTAYMQLSSLTSEDSAVYYCARSTYYGGDWYFNVWGAGTTVT VSAASTKGPSVFPLAPSSKSTSGGTAALGCLVKDYFPEPVTVSWNSGALTSGVHTFPAVLQS SGLYSLSSVVTVPSSSLGTQTYICNVNHKPSNTKVDKKVEPKSCDKTHTCPPCPAPELLGGPS VFLFPPKPKDTLMISRTPEVTCVVVDVSHEDPEVKFNWYVDGVEVHNAKTKPREEQYNSTY RVVSVLTVLHQDWLNGKEYKCKVSNKALPAPIEKTISKAKGQPREPQVYTLPPSREEMTKN QVSLSCAVKGFYPSDIAVEWESNGQPENNYKTTPPVLDSDGSFFLVSKLTVDKSRWQQGNV FSCSVMHEALHNRFTQKSLSLSPG (SEQ ID NO:34) or QVQLQQPGAELVKPGASVKMSCKASGYTFTSYNMHWVKQTPGRGLEWIGAIYPGNGDTS YNQKFKGKATLTADKSSSTAYMQLSSLTSEDSAVYYCARSTYYGGDWYFNVWGAGTTVT VSAASTKGPSVFPLAPSSKSTSGGTAALGCLVKDYFPEPVTVSWNSGALTSGVHTFPAVLQS SGLYSLSSVVTVPSSSLGTQTYICNVNHKPSNTKVDKKVEPKSCDKTHTCPPCPAPELLGGPS VFLFPPKPKDTLMISRTPEVTCVVVDVSHEDPEVKFNWYVDGVEVHNAKTKPREEQYNSTY RVVSVLTVLHQDWLNGKEYKCKVSNKALPAPIEKTISKAKGQPREPQVYTLPPSREEMTKN QVSLSCAVKGFYPSDIAVEWESNGQPENNYKTTPPVLDSDGSFFLVSKLTVDKSRWQQGNV FSCSVMHEALHNRFTQKSLSLSPGK (SEQ ID NO:35) and an antibody light chain polypeptide comprising the amino acid sequence of QIVLSQSPAILSASPGEKVTMTCRASSSVSYIHWFQQKPGSSPKPWIYATSNLASGVPVRESG SGSGTSYSLTISRVEAEDAATYYCQQWTSNPPTFGGGTKLEIKRTVAAPSVFIFPPSDEQLKS GTASVVCLLNNFYPREAKVQWKVDNALQSGNSQESVTEQDSKDSTYSLSSTLTLSKADYE KHKVYACEVTHQGLSSPVTKSFNRGEC (SEQ ID NO:36). In some embodiments, the antigen binding site that binds CD20 comprises the VH and VL domain sequences from obinituzumab.

In some embodiments, the multspecific antibody comprises one or more linker sequences, e.g., between a constant and variable domain, between a variable and constant domain, or otherwise shown in the formulas supra. In some embodiments, a linker sequence comprises one or more glycine and/or serine residue(s). In some embodiments, a linker sequence comprises one or more repeats of the sequence GGGGS (SEQ ID NO:104), e.g., one, two, three, four, five, or more than five repeats. Linkers for creating antibodies and antibody fusion proteins are known in the art. In some embodiments, the linker comprises, consists of, or consists essentially of, glycine and/or serine residues. In some embodiments, the linker is 15-20 amino acids in length. In some embodiments, the linker comprises the sequence GGGSGGGSGGGS (SEQ ID NO:105). In some embodiments, the linker comprises one or more repeats of the sequence GGGGS (SEQ ID NO:104). In some embodiments, the linker comprises the sequence GGGGSGGGGSGGGGS (SEQ ID NO:106) or GGGGSGGGGSGGGGSGGGGS (SEQ ID NO:107). Additional linker sequences are described in Chen, X. et al. (2013) Adv. Drug Deliv. Rev. 65:1357-1369. In some embodiments, a multispecific antibody of the present disclosure comprises two or more different types of linker sequence. In some embodiments, the linker comprises the sequence EPKRSDKTHTCPPC (SEQ ID NO:103) or SATHTCPPC (SEQ ID NO:108). In some embodiments, the linker comprises glycine and/or serine residues and is 15-20 amino acids in length.

In some embodiments, provided herein is a polynucleotide encoding the multispecific antibody of any one of the embodiments described herein. In some embodiments, provided herein is a vector (e.g., an expression vector) comprising the polynucleotide of any one of the embodiments described herein. In some embodiments, provided herein is a host cell (e.g., an isolated host cell or cell line) comprising the polynucleotide or vector of any one of the embodiments described herein. In some embodiments, provided herein is a pharmaceutical composition comprising the multispecific antibody of any one of the embodiments described herein and a pharmaceutically acceptable carrier. Any of these may find use in the methods of production and/or treatment disclosed herein.

In some embodiments, provided herein is a method of producing a multispecific antibody, comprising culturing the host cell of any one of the embodiments described herein under conditions suitable for production of the multispecific antibody. In some embodiments, the method further comprises recovering the multispecific antibody. The multispecific antibodies may be produced using standard recombinant techniques, as described herein, and/or as exemplified infra. In some embodiments, recovering the multispecific antibody comprises contacting the multispecific antibody with Protein A and eluting the multispecific antibody from the Protein A using a buffer comprising 3M MgCl₂. In some embodiments, prior to production of the multispecific antibody, the host cell is treated with kifunensine.

Antibodies and antibody fragments may be produced using recombinant methods. For example, nucleic acid encoding the antibody/fragment can be isolated and inserted into a replicable vector for further cloning or for expression. DNA encoding the antibody/fragment may be readily isolated and sequenced using conventional procedures (e.g., via oligonucleotide probes capable of binding specifically to genes encoding the heavy and light chains of the antibody/fragment). Many vectors are known in the art; vector components generally include, but are not limited to, one or more of the following: a signal sequence, an origin of replication, one or more marker genes, an enhancer element, a promoter, and a transcription termination sequence. Suitable host cells for cloning or expressing the DNA in the vectors herein are the prokaryote, yeast, or higher eukaryote cells. When using recombinant techniques, the antibody/fragment can be produced intracellularly, in the periplasmic space, or directly secreted into the medium. If the antibody/fragment is produced intracellularly, the particulate debris, either host cells or lysed fragments, are removed, for example, by centrifugation or ultrafiltration. Where the antibody/fragment is secreted into the medium, supernatants from such expression systems are generally first concentrated using a commercially available protein concentration filter.

In some embodiments, a multispecific antibody of the present disclosure is part of a pharmaceutical composition, e.g., including the antibody and one or more pharmaceutically acceptable carriers. Pharmaceutical compositions and formulations as described herein can be prepared by mixing the active ingredients (such as a fusion protein) having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

### II. Methods of Use

In certain aspects, the present disclosure provides methods of treating a disease or disorder, comprising administering an effective amount of a multispecific antibody or composition of the present disclosure to an individual in need thereof. In some embodiments, the individual is a human. In some embodiments, the individual has been diagnosed with a disease or disorder, including without limitation cancer, a bacterial infection, a fungal infection, a viral infection, a mast cell disease or disorder, systemic mastocytosis, amyloidosis, or an aging-related disease or disorder

Any of the multispecific antibodies of the present disclosure (e.g., as described supra in section I) may find use in the methods of treatment and uses disclosed herein, as well as the compositions (e.g., pharmaceutical compositions) related thereto. For example, in some embodiments, the methods include using a multispecific antibody or composition of the present disclosure with a first antigen binding domain that binds to human Dectin-1, and a second antigen binding domain that binds to a disease-causing agent. In some embodiments, the disease-causing agent is a bacterial cell, fungal cell, virus, senescent cell, tumor cell, protein aggregate (e.g., amyloid beta, or lambda or kappa light chain amyloids), LDL particle, mast cell, eosinophil, ILC2 cell, or inflammatory immune cell. In some embodiments, the target of interest is an antigen expressed on the surface of the bacterial cell, fungal cell, senescent cell, tumor cell, mast cell, eosinophil, ILC2 cell, or inflammatory immune cell. In some embodiments, the target of interest is a surface antigen of the virus. In some embodiments, the target of interest is CD70, HER2, DLL3, NECTIN-4, TROP-2, Mesothelin, LIV-1, C-MET, FOLR1, CD20, CCR8, CD33, or EGFR. Binding of the antibody that mediates targeted removal of a disease-causing agent via phagocytosis could be with and without avidity i.e., with and without inducing dimerization of the phagocytosis receptor such as Dectin-1 or the target antigen present on the agent.

In some embodiments, the disease or disorder is cancer, a bacterial infection, a fungal infection, a viral infection, a mast cell disease or disorder, systemic mastocytosis, amyloidosis, or an aging-related disease or disorder. There are variety of accumulated and not cleared aberrant host cells such as tumor, lymphoma, dead, necrotic, apoptotic, dying, infected, damaged cells that are associated with diseases. In addition, diverse cell products such as aggregated proteins (β-amyloid plaque, Tau aggregates, or antibody lambda or kappa light chain amyloids), lipoprotein particles, could cause a disease upon increased accumulation. Disease-causing cell may have glycoprotein, surface protein, or glycolipid typical of aberrant cells associated with a disease, disorder, or other undesirable condition. Besides the host generated agents, variety of foreign pathogens such as infectious microbes (e.g., viruses, fungus and bacteria) and the microbe generated products and debris (e.g., viral particle envelopes, endotoxin) may not be well cleared in patients. In some embodiments, the virus is an influenza virus. In some embodiments, the virus is SARS-CoV-2.

The above listed abnormalities may cause illnesses such as cancer, Alzheimer disease, fibrosis, Parkinson disease, Huntington disease, HIV, Hepatitis A, B or C, sepsis etc. Many of these disorders or diseases are characterized by an accumulation of disease-causing agents in different organs in human subjects. In addition to the beneficial removal of a disease-causing agent via phagocytosis, the molecule may induce production of inflammatory mediators to alter the disease microenviroment such as in tumors, cancers and lymphomas. Without wishing to be bound to theory, it is thought that the molecule that performs targeted phagocytosis may demonstrate clear benefits for patients such as Alzheimer disease, Parkinson disease, cancer, infectious diseases (viral, bacterial, fungal, protozoan infections), inflammatory, or immune diseases (e.g., autoimmune diseases, inflammatory bowel diseases, multiple sclerosis), degenerative disease (e.g., joint and cartilage) Rheumatoid arthritis, Felty's syndrome, aggressive NK leukemia, IBM, IBD etc. In addition, targeted phagocytosis antibody treatment may have better activity of depleting cells in tissues over ADCC that relies on NK cells. The treatment may have a selective activity for removal of a particular disease-causing agent over a therapy that targets myeloid cells and improves phagocytosis in general. For example, targets of interest for treatment of cancer include, without limitation, CD70, HER2, DLL3, NECTIN-4, TROP-2, Mesothelin, LIV-1, C-MET, FOLR1, CD20, CCR8, CD33, and EGFR.

### III. Kits and Articles of Manufacture

Certain aspects of the present disclosure relate to kits or articles of manufacture comprising any of the multispecific antibodies disclosed herein. In some embodiments, the article of manufacture comprises a container and a label or package insert on or associated with the container. In some embodiments, the kit or article of manufacture further comprises instructions for using the multispecific antibody according to any of the methods disclosed herein, e.g., for treating a disease or disorder such as cancer.

Suitable containers include, for example, bottles, vials, syringes, etc. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). At least one active agent in the composition is an antibody or multispecific binding molecule as described herein. The label or package insert indicates that the composition is used for treating the particular condition. The label or package insert will further comprise instructions for administering the multispecific antibody or composition to the subject. Articles of manufacture and kits comprising combinatorial therapies described herein are also contemplated.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Bispecific Antibodies with a Designed Heterodimerization Interface

This example describes a bispecific antibody design that promotes correct heterodimerization between half-antibody arms (i.e., two distinct heavy chains) and between heavy and light chains.

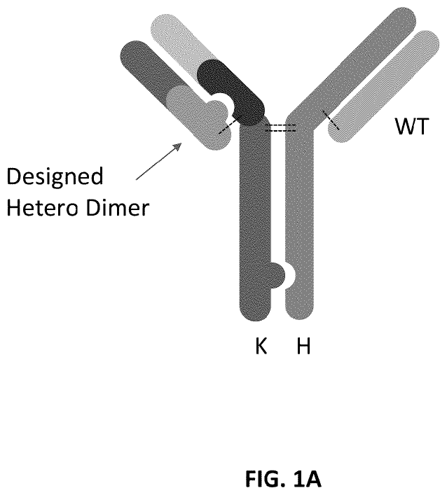

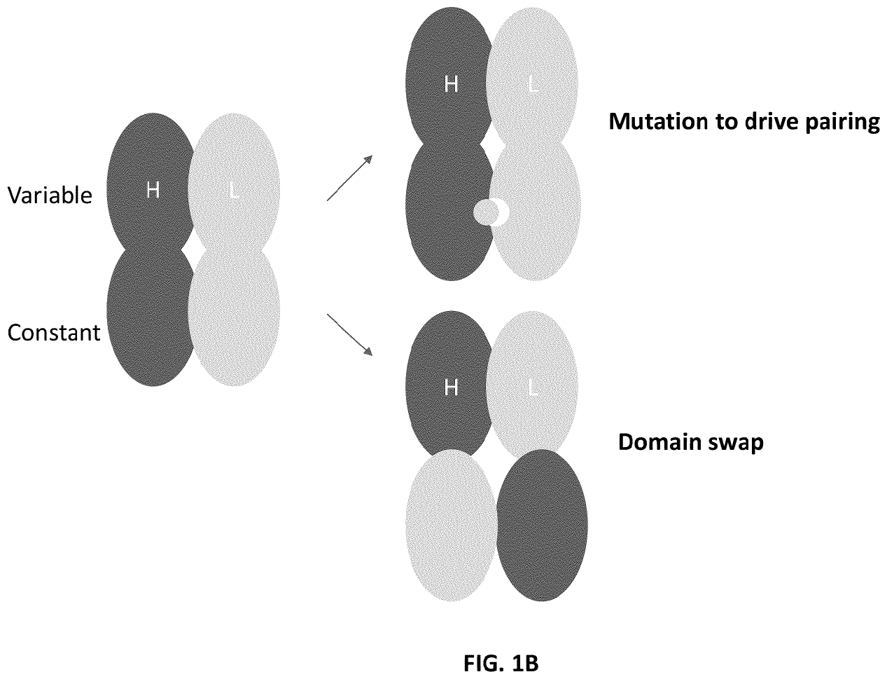

Bispecific antibody designs allow for the targeting of multiple factors with a single molecule. A diagram of an exemplary bispecific antibody is shown in FIG. 1A, with a conventional wild-type (WT) arm (e.g., targeting an immune cell), paired with another arm (e.g., targeting a disease antigen) containing a designed heterodimer domain to facilitate correct chain association. For such a bispecific design, 4 individual subunits must pair correctly, allowing for multiple VH/VL combinations.

The correct association of heavy chains and light chains can be promoted by knobs-into-holes technology (introducing knob-forming and hole-forming mutations into native constant domains) or domain cross-over (swapping CH1 and C_{L} domains between heavy and light chains, e.g., CrossMab; see US PG Pub. No. 2017/0129962) as shown in FIG. 1B. Other domain swapping approaches to promote correct heavy:light chain pairing include replacement of the CH1 and CL domains with antibody CH2 domains (see, e.g., U.S. PG Pub. No. 2018/0057567), antibody CH3 domains (see, e.g., U.S. Pat. No. 10,982,008), or TCR constant domains (see, e.g., U.S. PG Pub. No. 2020/0283524); or swapping positions of the CH1 and C_{L} domains (see, e.g., U.S. PG Pub. No. 2017/0129962). For domain cross-over, pairing can be further enhanced by subsequent introduction of knob-forming and hole-forming mutations into the constant domains. Engineered disulfide bonds can also be introduced to promote correct pairing; see, e.g., U.S. Pat. No. 9,527,927.

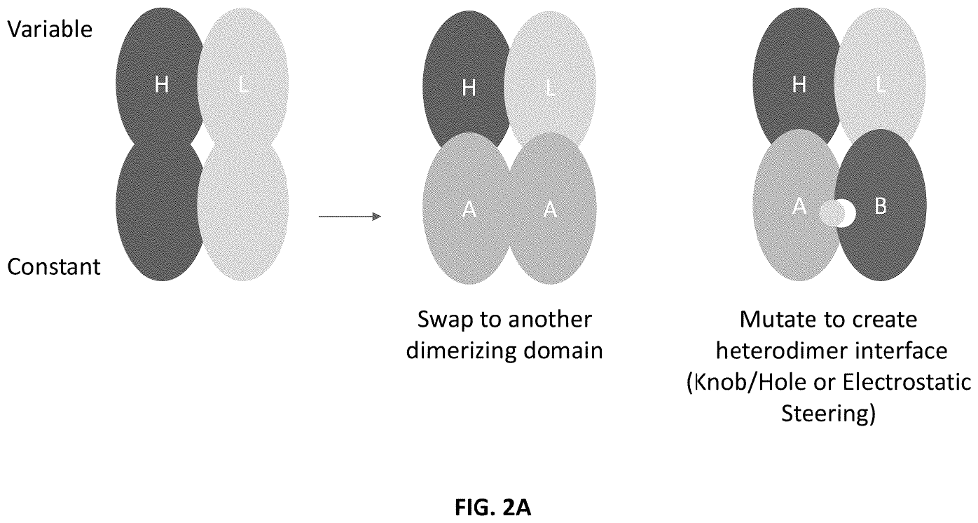

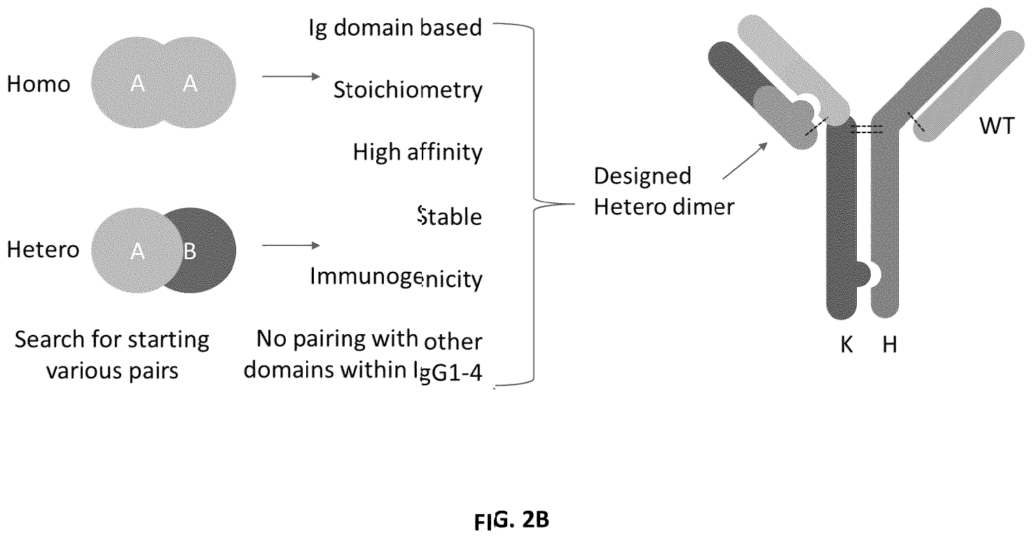

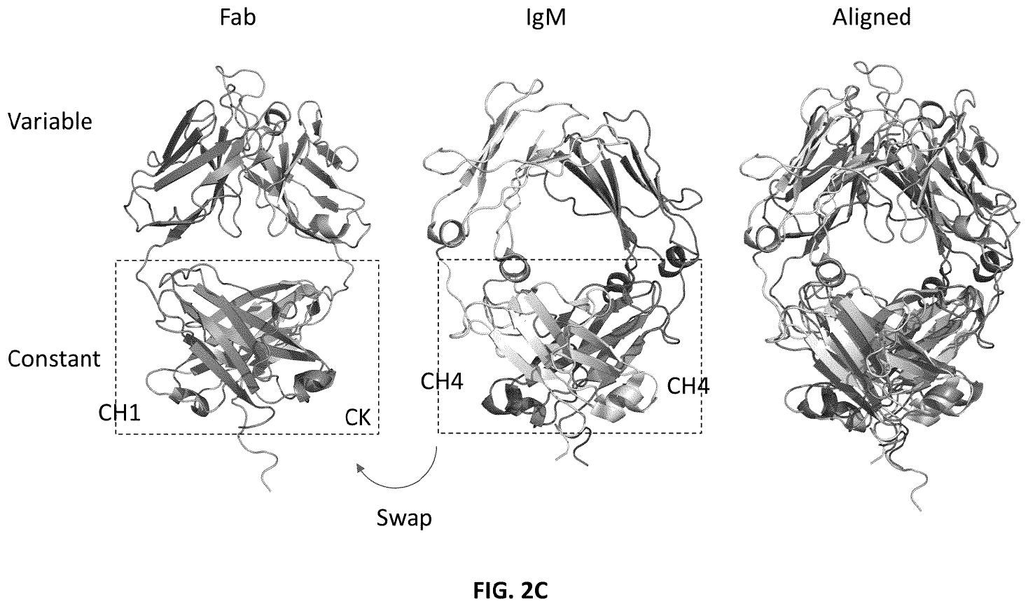

A combination of domain cross-over and knobs-into-holes technology can be used to create a mutated heterodimer interface that promotes correct association between heavy and light chains. As shown in FIG. 2A, the constant domains can be swapped out for another dimerizing domain (e.g., the human T-cell receptor constant region in the WuXiBody platform; see, e.g., US PG Pub. No. 2020/0283524). However, this interface can be further mutated with knobs-into-holes technology or electrostatic steering to create a heterodimerization domain.

Several criteria are ideally satisfied in the selection of a suitable domain to be used for exchange/cross-over of the CH1 and C_{L} regions in an engineered bispecific antibody (FIG. 2B). An optimal heterodimer domain for this purpose would ideally be stable, non-immunogenic, Ig domain-based, stoichiometric, have a high affinity for its target, and would not pair with other domains within IgG1-4.

A search for a suitable domain that met the above criteria identified the CH4 domain of IgM. Structural comparison of the CH1:CK interface of a Fab fragment with the CH4: CH4 interface of IgM antibodies showed high similarity with a root mean squared deviation (RMSD) of 2.041 Å, suggesting swapping of CH1 and/or CK with an IgM CH4 domain would be compatible (FIG. 2C). Advantageously, using a CH4 domain would reduce the likelihood that the domain for promoting heterodimerization would instead pair with a domain of the antibody constant or Fc region, such as a CH1, CH2, or CH3 domain.

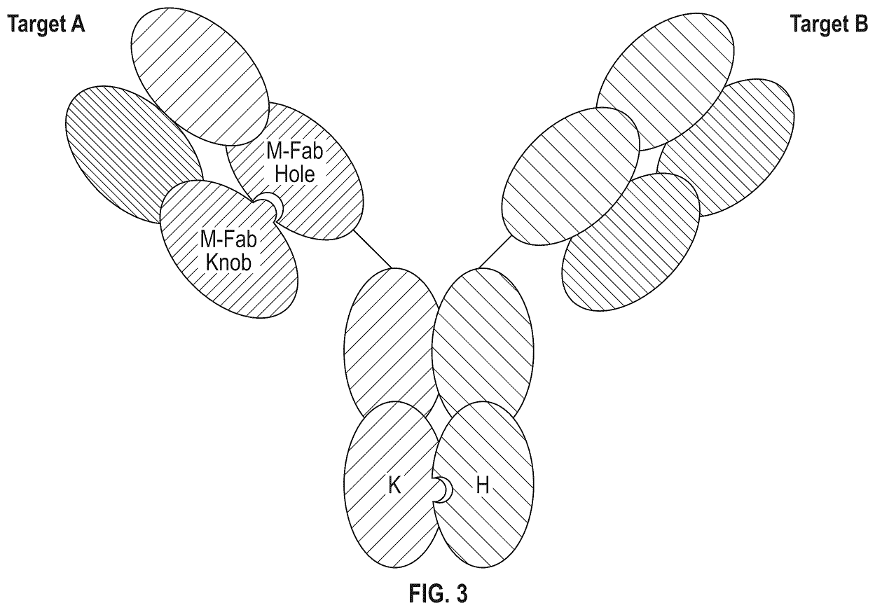

A diagram of an exemplary bispecific antibody that incorporates the above domain swapping and engineering approaches, M-Fab 2M24xCD20, is shown in FIG. 3. The antibody has an anti-CD20 rituximab (RTX) arm and an anti-Dectin-1 2M24 arm. Domain swapping is used to promote dimerization of the 2M24 heavy and light chains. Knobs-into-holes technology is used to enhance pairing of the two distinct heavy chains between the half-antibody arms, and both domain cross-over (with the constant domain of IgM) and knobs-into-holes technology is used to enhance pairing of the heavy and light chains in the 2M24 arm.

### Example 2: Functional Characterization of the M-Fab 2M24xCD20 Antibody

This example describes the generation, purification, and functional characterization of bispecific antibodies comprising a 2M24 Dectin-1-binding arm and a rituximab CD20-binding arm.

### Materials and Methods

### Generation of Bispecific Antibodies

Bispecific antibodies were expressed by transfecting 4 plasmids with individual subunits (2 light chains and 2 heavy chains) into HEK293 cells. Supernatant was harvested after four days of expression

### Antibody Purification

Sample was loaded on a column packed with 5 mL MabSelect PrismA that was pre-equilibrated with Buffer A (50 mM MES pH 6.5, 150 mM NaCl). Captured antibody was washed and eluted using 0-100% gradient Buffer B (50 mM MES pH 6.5, 3 M MgCl₂). Eluted fractions were pooled and buffer exchanged into Buffer A.

### SEAP Reporter Assay in HEK-Blue Cells Expressing Dectin-1

To determine HEK cell SEAP secretion induced by Raji cells (expressing CD20), Raji cells were mixed at a 1:1 ratio with HEK-Blue hDectin-1-a cells in DMEM with 10% heat-inactivated FBS. Titrated antibody, either M-Fab (2M24xCD20 hG1M), DuetMab (2M24xCD20 hG1), or an isotype control (2M24xRSV hG1), was added to the cells. After 22 hours, alkaline phosphatase levels were assessed in the supernatant at OD630 using QUANTI Blue Solution (Invivogen, San Diego, CA) per manufacturer's instructions.

### Results

### Expression and Purification of the 2M24xCD20 M-Fab Antibody

M-Fab (2M24xCD20 hIgG1 M) (FIG. 3) was expressed by transfecting 4 plasmids with individual subunits (CD20 heavy chain, CD20 light chain, 2M24 heavy chain, 2M24 light chain) into HEK293 cells. Supernatant was harvested after four days of expression and purified via Protein A. Aggregates were removed with size exclusion chromatography (SEC).

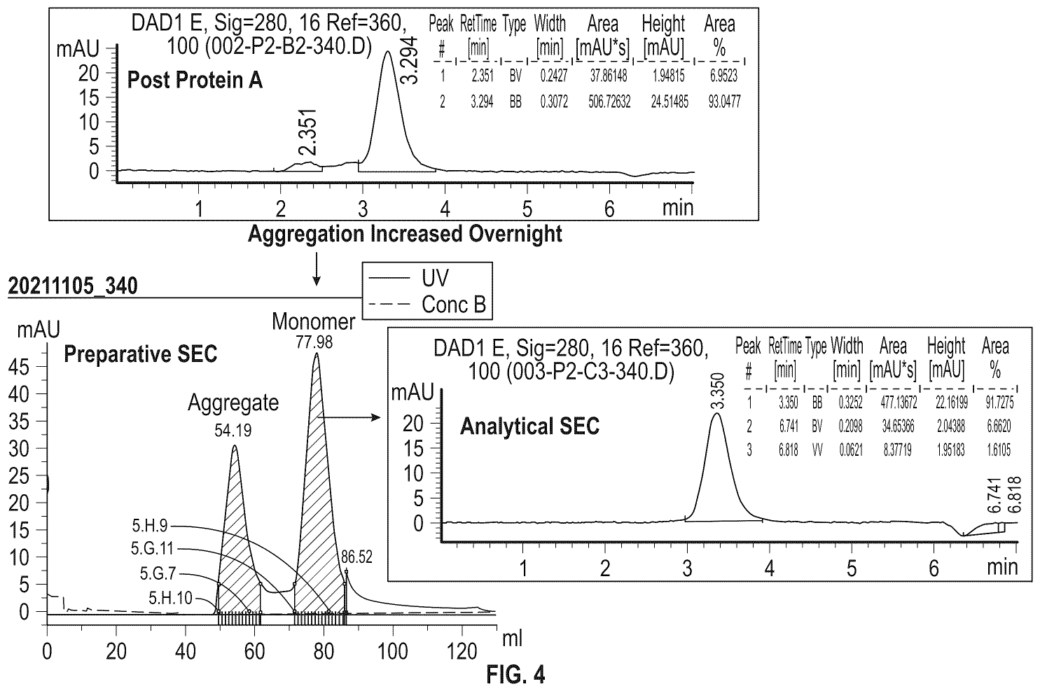

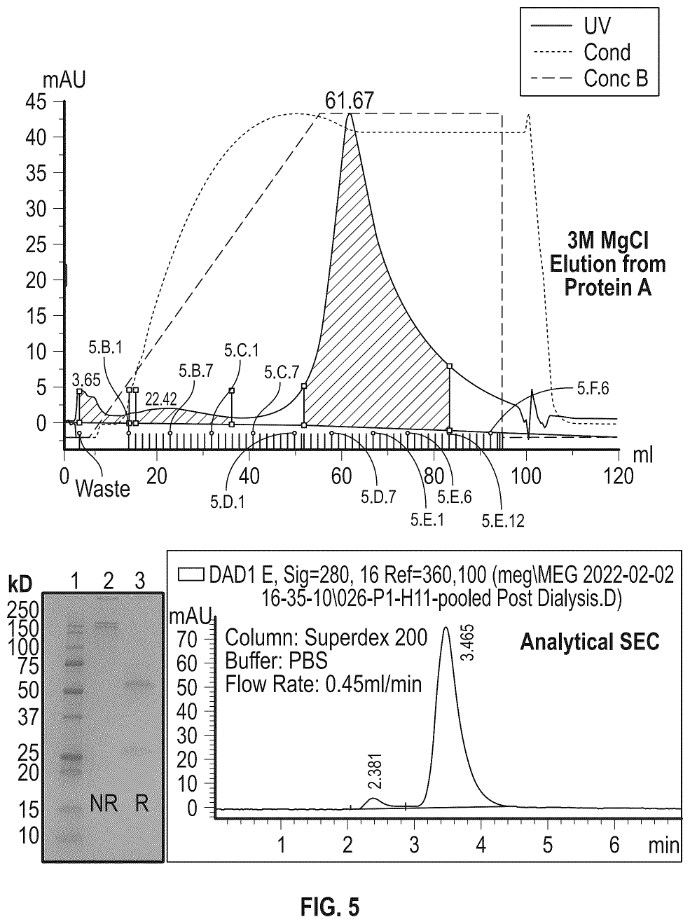

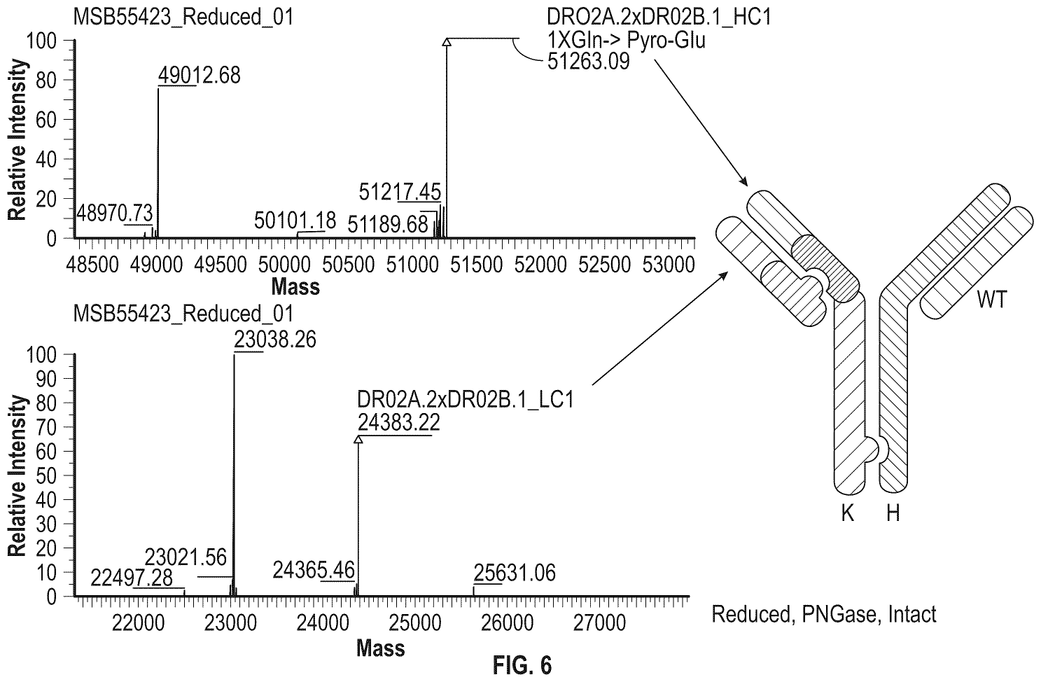

The M-Fab bispecific antibody tended to aggregate under standard conditions for pH elution of antibodies, and this was further exacerbated overnight. After protein A purification, there is a fraction that is predominantly monomer, but also some aggregation. Elution with 3M MgCl₂ helped resolve this aggregation. The M-Fab bispecific antibody purified as a homogenous molecule on SEC with 93% of antibody as a monomer after elution and was stable in PBS over 48 h at 4° C. (FIG. 4). The purity of the M-Fab antibody was assessed by SDS-PAGE analysis under non-reducing and reducing conditions after elution with a gradient using 3M MgCl₂, yielding 0.53 mg/mL bispecific in 50 mM MES pH 6.5 with 150 mM NaCl (FIG. 5). Mass spectrometry (MS) analysis confirmed the presence of the IgM domain swap into the heavy and light chains of the 2M24 half-antibody arm in M-Fab (FIG. 6).

### Binding Affinity of the 2M24xCD20 Antibody

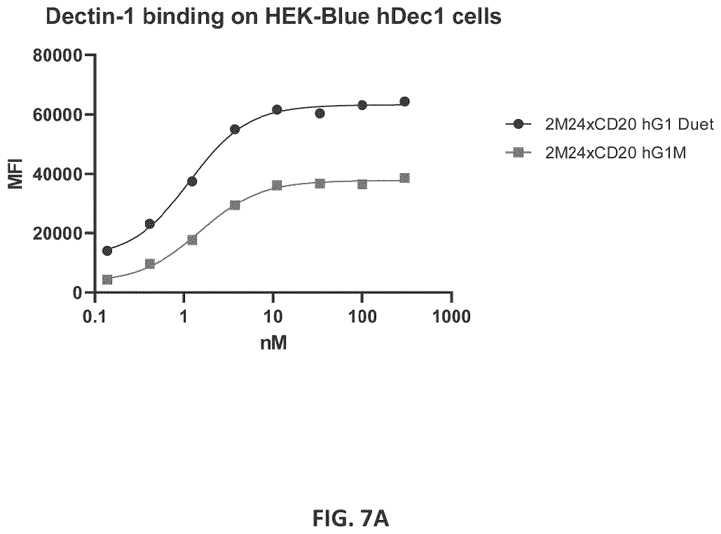

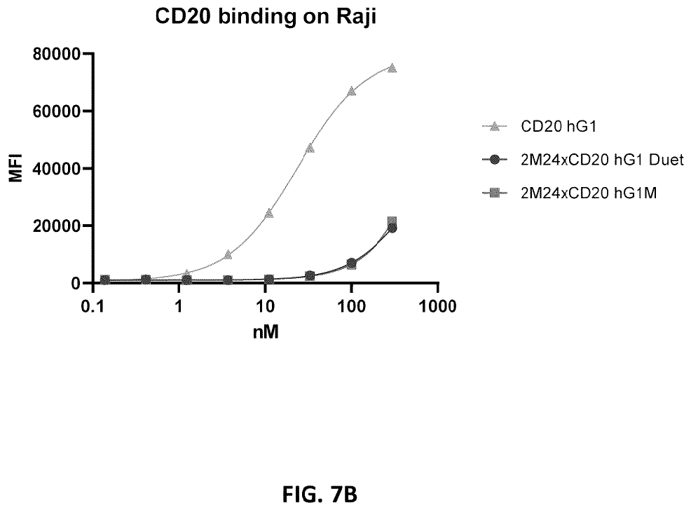

To evaluate the effect of the 2M24xCD20 bispecific antibody on cells, its binding affinity for cells expressing Dectin-1 or CD20 was measured. Two different formats were used to drive heavy:light chain pairing and bispecific antibody assembly: the DuetMab format with engineered disulfide bonds (see Mazor, Y. et al. (2015) MAbs 7:377-389) (“2M24xCD20 hG1 Duet”) and the M-Fab format described herein (see, e.g., FIG. 3) (“2M24xCD20 hG1M”), as compared to monospecific anti-CD20 with hG1 Fc. Binding of the bispecific 2M24xCD20 antibodies to Dectin-1-expressing HEK293 cells was assessed by flow cytometry (FIG. 7A). Both M-Fab and DuetMab formats were able to bind cells expressing Dectin-1 with similar affinities. Binding of the bispecific 2M24xCD20 antibodies to cells expressing CD20 was also assessed by flow cytometry, using the CD20-expressing B cell lymphoma Raji cell line (FIG. 7B). Both M-Fab and DuetMab were able to bind cells expressing CD20 with similar affinities, but with reduced affinity compared to the parental monospecific anti-CD20 antibody.

### SEAP Reporter Assay for Dectin-1 Signaling Induction by the 2M24xCD20 Antibody

The M-Fab 2M24xCD20 antibody was also tested for its ability to promote signaling through Dectin-1, using the secreted alkaline phosphatase (SEAP) reporter assay. In this assay, increasing concentrations of bispecific antibodies are co-cultured with CD20-expressing Raji cells and HEK-Blue cells engineered to express Dectin-1. A bispecific antibody that connects an immune cell (e.g., a macrophage expressing Dectin-1) with a disease target (e.g., CD20 antigen) will specifically induce gene expression in the Dectin-1/NF-κB/SEAP signaling pathway, wherein activating ligands for Dectin-1 promote downstream activation of NF-κB and subsequent SEAP secretion into the extracellular media. SEAP release can therefore be used as a readout for Dectin-1 activation, approximating immunostimulatory effects induced by bispecific antibody binding.

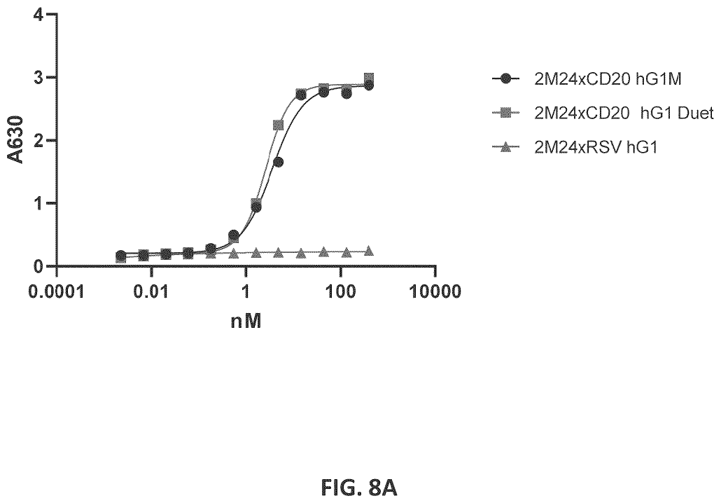

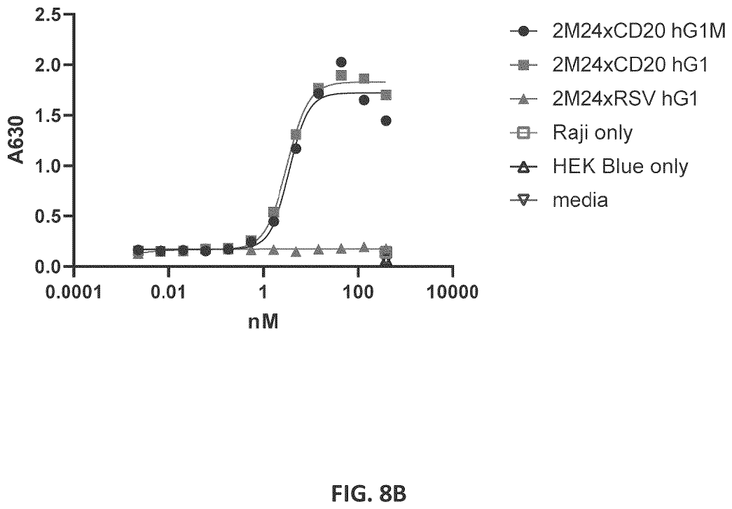

Co-cultures of CD20-expressing Raji cells and the Dectin-1-expressing HEK-Blue cells were treated with increasing concentrations of M-Fab 2M24xCD20 bispecific, DuetMab 2M24xCD20 bispecific, or a bispecific isotype control (2M24xRSV hG1). As shown in FIGS. 8A-8B, M-Fab and DuetMab bispecific antibodies showed similar activity (M-Fab EC₅₀=3.65 nM (Exp. 1), 3.53 nM (Exp. 2); DuetMab EC₅₀=2.63 nM (Exp. 1), 3.19 nM (Exp. 2)), promoting Dectin-1 signaling and downstream NF-κB activity as estimated based on SEAP release into the media. These data indicate that, compared to DuetMab, the M-Fab bispecific antibody has similar immunostimulatory activity.

### Example 3: Characterization of the 2:1 Format M-Fab 2M24xTrop2 Bispecific Antibody

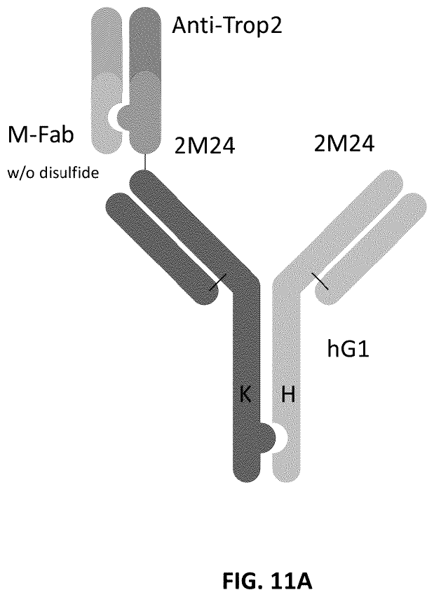

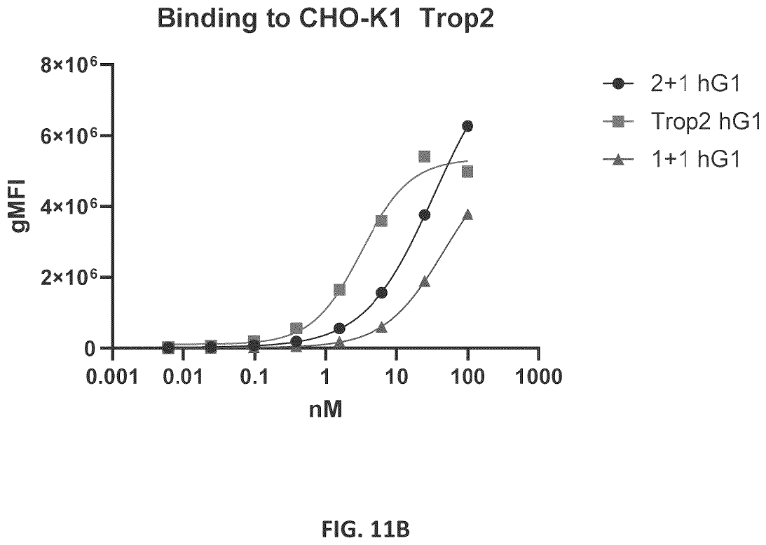

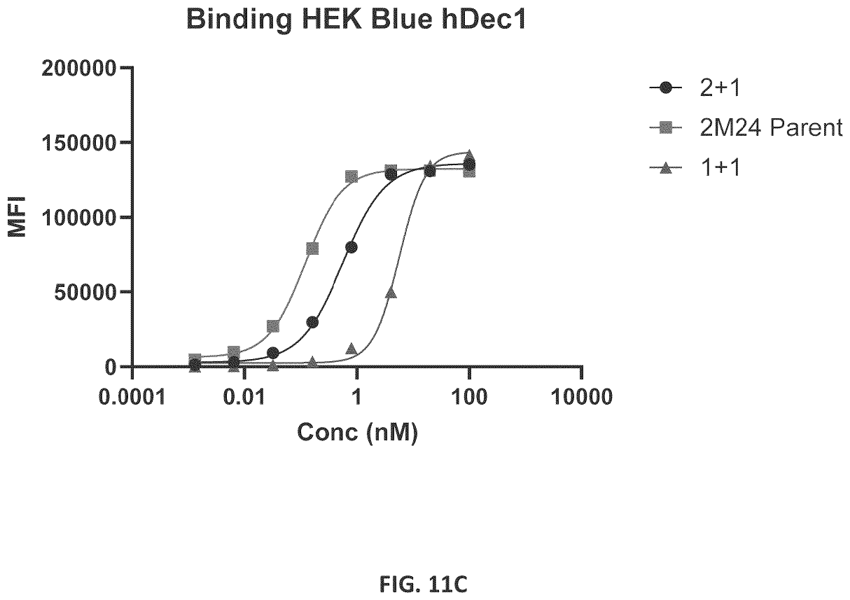

This example describes the generation, purification, and functional characterization of bispecific, trivalent antibodies comprising two 2M24 Dectin-1-binding sites and one anti-Trop2 binding site having the general structure illustrated in FIG. 11A.

2+1 M-Fab (2M24xTrop2 hIgG1) bispecific antibody (FIG. 11A) was expressed by transfecting 4 plasmids with individual subunits (2M24 heavy chain hole, 2M24 light chain, anti-Trop2+2M24 heavy chain knob, anti-Trop2 light chain MFab) into HEK293 cells. Supernatant was harvested after 5 days, clarified and 2+1 M-Fab bispecific was first isolated via Protein A beads, and followed with size exclusion chromatography to remove higher order species. Amino acid sequences for each polypeptide were as follows: monovalent heavy chain: SEQ ID NO:41; 2M24 light chain: SEQ ID NO:42; bivalent/bispecific heavy chain: SEQ ID NO:38; anti-Trop2 light chain: SEQ ID NO: 39.

2+1 M-Fab (2M24xTrop2 hIgG1) was tested for the ability to bind Trop-2 as expressed by CHO-K1 cells and human Dectin-1 as expressed by HEK-Blue hDectin-1-a cells. As shown in FIGS. 11B & 11C, 2+1 M-Fab (2M24xTrop2 hIgG1) was able to bind cells expressing Trop2 or Dectin-1 similar to traditional bispecific antibody. Notably, the EC50 of 2+1 M-Fab (2M24xTrop2 hIgG1) binding to hDec1-expressing cells was shifted left as compared to traditional bispecific antibody (FIG. 11C).

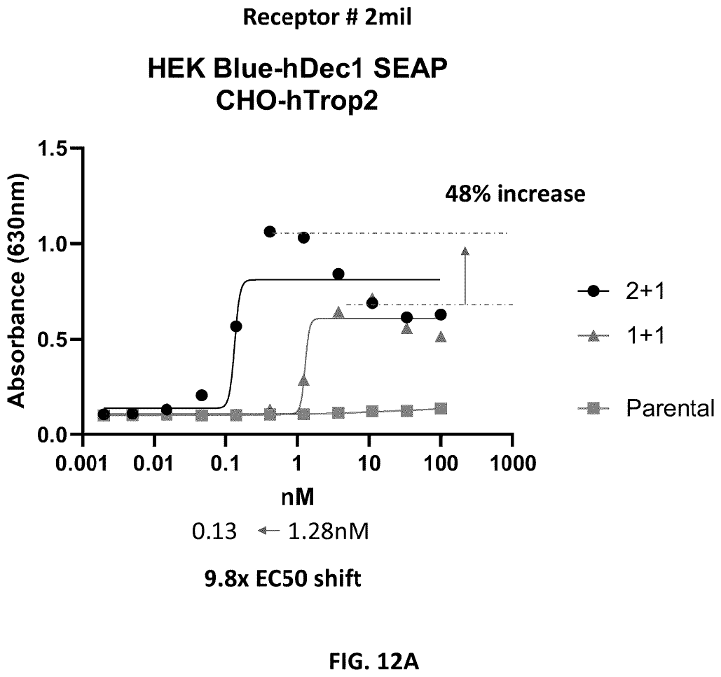

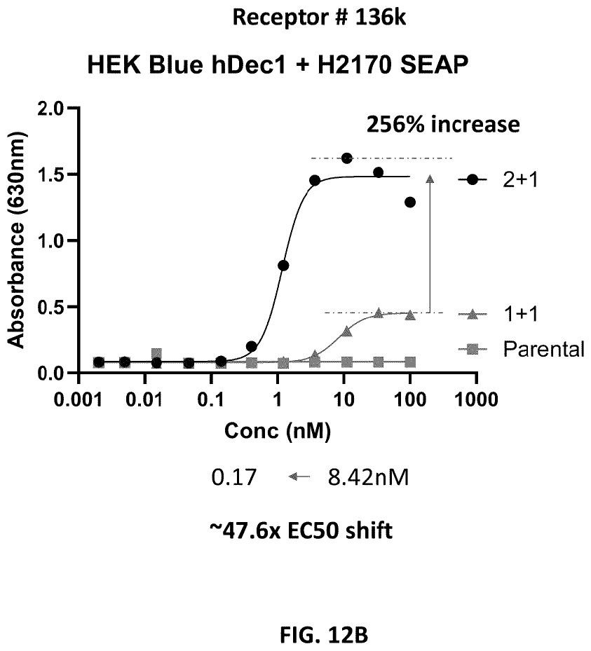

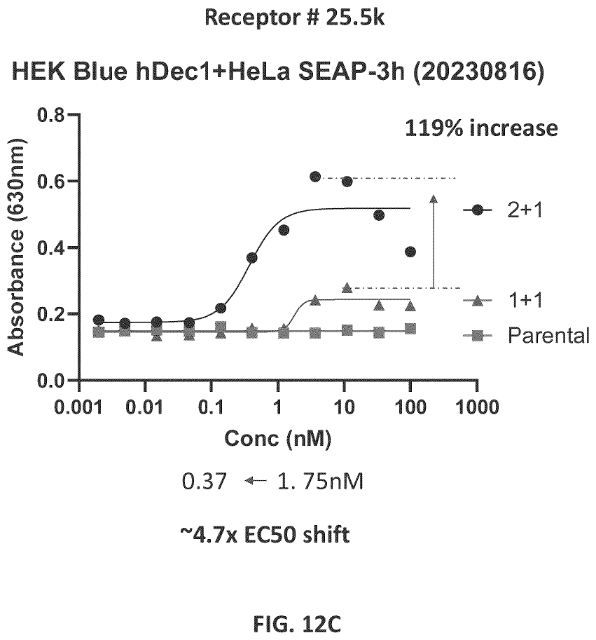

The ability of 2+1 M-Fab (2M24xTrop2 hIgG1) to activate Dectin-1 signaling in the presence of Trop2-expressing cells was tested in the SEAP reporter assay described in Example 2. As shown in FIG. 12A, 2+1 M-Fab (2M24xTrop2 hIgG1) showed increased activation of Dectin-1 signaling as compared to traditional bispecific antibody in the presence of CHO cell lines overexpressing Dectin-1 at approximately 2 million copies/cell. This was evident in a 48% increase in reporter output (as measured by Abs_{630 nm}) and a 9.8-fold shift in EC50 from 1.28 nM for traditional bispecific to 0.13 nM for 2+1 M-Fab (2M24xTrop2 hIgG1). To test these effects using cells expressing Trop2 at lower levels closer to endogenous expression, H2170 cells expressing Trop2 (˜136,000 copies/cell) and HeLa cells expressing Trop2 (˜25,500 copies/cell) were used. As shown in FIG. 12B, increased activation of Dectin-1 signaling in presence of H2170 cells by 2+1 M-Fab (2M24xTrop2 hIgG1) as compared to traditional bispecific antibody was again observed with a 256% increase in Abs_{630 nm} and a 47.6-fold EC50 shift from 8.42 nM to 0.17 nM. As shown in FIG. 12C, increased activation of Dectin-1 signaling in presence of HeLa cells by 2+1 M-Fab (2M24xTrop2 hIgG1) as compared to traditional bispecific antibody was again observed with a 119% increase in Abs_{630 nm} and a 4.7-fold EC50 shift from 1.75 nM to 0.37 nM. These results demonstrate more potent activation of Dectin-1 signaling by the 2+1 M-Fab (2M24xTrop2 hIgG1) bispecific antibody as compared to traditional 2M24/Trop2 bispecific antibody in the presence of cells expressing a wide range of Trop2 levels.

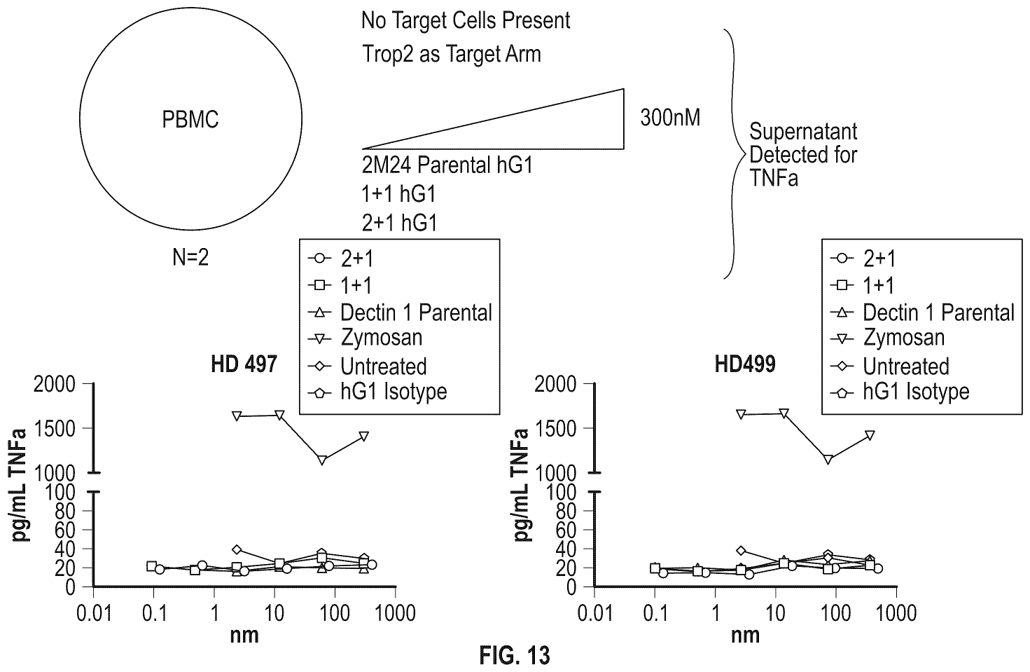

To test for off-target effects of 2+1 M-Fab (2M24xTrop2 hIgG1) in the absence of target cells, peripheral blood mononuclear cells (PBMCs) were isolated from the blood of 2 donors, and various antibodies (2+1 M-Fab 2M24xTrop2 hIgG1, traditional 2M24/Trop-2 bispecific antibody, and parental 2M24 antibody) and controls were incubated with the PBMCs overnight at varying concentrations. This included zymosan, a Dectin-1 agonist, as a positive control and untreated cells or hIgG1 isotype control as negative controls. After incubation, supernatant was analyzed for TNF-alpha release as an indicator of Dectin-1 agonism. As shown in FIG. 13, neither 2+1 M-Fab (2M24xTrop2 hIgG1) nor traditional bispecific antibody led to off-target activation of Dectin-1 in the absence of target cells (i.e., expressing Trop2) at the concentrations tested.

Although the present disclosure has been described in some detail by way of illustration and example for purposes of clarity of understanding, the descriptions and examples should not be construed as limiting the scope of the present disclosure. The disclosures of all patent and scientific literature cited herein are expressly incorporated in the entirety by reference.

1. A multispecific antibody, comprising:
a) a first arm comprising:
i) a first polypeptide that comprises, in an N-terminal to C-terminal direction, a structure represented by the formula: 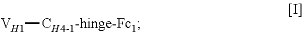
and
ii) a second polypeptide that comprises, in the N-terminal to C-terminal direction, a structure represented by the formula: 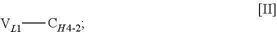
and
b) a second arm comprising:
iii) a third polypeptide that comprises, in the N-terminal to C-terminal direction, a structure represented by the formula: 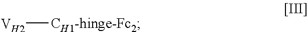
and
iv) a fourth polypeptide that comprises, in the N-terminal to C-terminal direction, a structure represented by the formula: 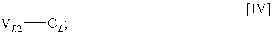
wherein:
V_{H1} is a first heavy chain variable (VH) domain;
V_{H2} is a second VH domain;
V_{L1} is a first light chain variable (VL) domain;
V_{L2} is a second VL domain;
C_{H4-1} is a first antibody constant heavy chain 4 (CH4) domain;
C_{H4-2} is a second antibody CH4 domain;
hinge is an antibody hinge region;
C_{H1} is an antibody constant heavy chain 1 (CH1) domain;
Fc₁ is a first antibody Fc region;
Fc₂ is a second antibody Fc region; and
C_{L} is an antibody light chain constant (CL) domain;
wherein the first VH domain and the first VL domain form a first antigen binding site that specifically binds a first antigen, and the second VH domain and the second VL domain form a second antigen binding site that specifically binds a second antigen;
wherein the first and/or second antibody CH4 domains comprise at least one amino acid substitution that promotes association of the first and second antibody CH4 domains; and
wherein the first and/or second antibody Fc regions comprise at least one amino acid substitution that promotes association of the first and second antibody Fc regions.

2. The multispecific antibody of claim 1, wherein the first and second antibody CH4 domains are human IgM CH4 domains or human IgE CH4 domains.

3. The multispecific antibody of claim 2, wherein the first and/or second antibody CH4 domains each comprise at least one amino acid substitution relative to the amino acid sequence of SEQ ID NO: 1 or 4 that promotes association of the first and second antibody CH4 domains.

4-15. (canceled)

16. The multispecific antibody of claim 1, wherein the first and second antibody Fc regions are human IgG Fc regions.

17-23. (canceled)

24. The multispecific antibody of claim 1, wherein the first and second antigens are different.

25. The multispecific antibody of claim 24, wherein the first or the second antigen binding site specifically binds human Dectin-1.

26-55. (canceled)

56. The multispecific antibody of claim 1, wherein the first polypeptide comprises, in the N-terminal to C-terminal direction, a structure represented by the formula: 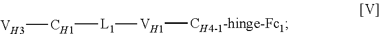
and
the multispecific antibody further comprises a fifth polypeptide that comprises, in the N-terminal to C-terminal direction, a structure represented by the formula: 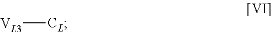
wherein:
V_{H3} is a third VH domain;
V_{L3} is a third VL domain;
C_{H1} is an antibody constant heavy chain 1 (CH1) domain;
L₁ is a linker sequence;
V_{H1} is the first VH domain;
C_{H4-1} is the first antibody CH4 domain;
hinge is the antibody hinge region;
Fc₁ is the first antibody Fc region; and
C_{L} is an antibody C_{L} domain;
wherein the third VH domain and the third VL domain form a third antigen binding site that specifically binds a third antigen.

57-68. (canceled)

69. The multispecific antibody of claim 56, wherein the third antigen binding site specifically binds human Dectin-1.

70. A multispecific antibody, comprising:
a) a first arm comprising:
i) a first polypeptide that comprises, in an N-terminal to C-terminal direction, a structure represented by the formula: 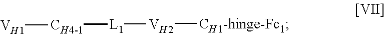
ii) a second polypeptide that comprises, in the N-terminal to C-terminal direction, a structure represented by the formula: 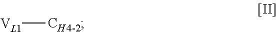
and
iii) a third polypeptide that comprises, in the N-terminal to C-terminal direction, a structure represented by the formula: 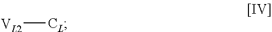
b) a second arm comprising:
iv) a fourth polypeptide that comprises, in the N-terminal to C-terminal direction, a structure represented by the formula: 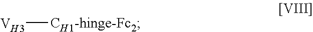
and
v) a fifth polypeptide that comprises, in the N-terminal to C-terminal direction, a structure represented by the formula: 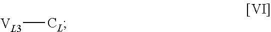
wherein:
V_{H1} is a first heavy chain variable (VH) domain;
V_{H2} is a second VH domain;
V_{H3} is a third VH domain;
V_{L1} is a first light chain variable (VL) domain;
V_{L2} is a second VL domain;
V_{L3} is a third VL domain;
L₁ is a linker sequence;
C_{H4-1} is a first antibody constant heavy chain 4 (CH4) domain;
C_{H4-2} is a second antibody CH4 domain;
C_{H1} is an antibody constant heavy chain 1 (CH1) domain;
hinge is an antibody hinge region;
Fc₁ is a first antibody Fc region;
Fc₂ is a second antibody Fc region; and
C_{L} is an antibody light chain constant (CL) domain;
wherein the first VH domain and the first VL domain form a first antigen binding site that specifically binds a first antigen, the second VH domain and the second VL domain form a second antigen binding site that specifically binds a second antigen, and the third VH domain and the third VL domain form a third antigen binding site that specifically binds a third antigen;
wherein the first and/or second antibody CH4 domains comprise at least one amino acid substitution that promotes association of the first and second antibody CH4 domains; and
wherein the first and/or second antibody Fc regions comprise at least one amino acid substitution that promotes association of the first and second antibody Fc regions.

71. The multispecific antibody of claim 70, wherein the first and second antibody CH4 domains are human IgM CH4 domains or human IgE CH4 domains.

72. The multispecific antibody of claim 71, wherein the first and/or second antibody CH4 domains each comprise at least one amino acid substitution relative to the amino acid sequence of SEQ ID NO: 1 or 4 that promotes association of the first and second antibody CH4 domains.

73-84. (canceled)

85. The multispecific antibody of claim 70, wherein the first and second antibody Fc regions are human IgG Fc regions.

86-91. (canceled)

92. The multispecific antibody of claim 70, wherein the second and third antigens are the same.

93. (canceled)

94. The multispecific antibody of claim 92, wherein the second and third antigen binding sites independently and specifically bind human Dectin-1, or wherein the first antigen binding site specifically binds human Dectin-1.

95-130. (canceled)

131. A multispecific antibody, comprising:
a) a first arm comprising:
i) a first polypeptide that comprises, in an N-terminal to C-terminal direction, a structure represented by the formula: 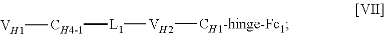
ii) a second polypeptide that comprises, in the N-terminal to C-terminal direction, a structure represented by the formula: 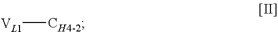
and
iii) a third polypeptide that comprises, in the N-terminal to C-terminal direction, a structure represented by the formula: 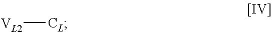
and
b) a second arm comprising:
iv) a fourth polypeptide that comprises, in the N-terminal to C-terminal direction, a structure represented by the formula: 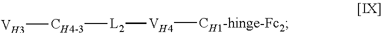
v) a fifth polypeptide that comprises, in the N-terminal to C-terminal direction, a structure represented by the formula: 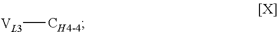
and
vi) a sixth polypeptide that comprises, in the N-terminal to C-terminal direction, a structure represented by the formula: 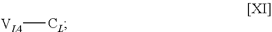
wherein:
V_{H1} is a first heavy chain variable (VH) domain;
V_{H2} is a second VH domain;
V_{H3} is a third VH domain;
V_{H4} is a fourth VH domain;
V_{L1} is a first light chain variable (VL) domain;
V_{L2} is a second VL domain;
V_{L3} is a third VL domain;
V_{L4} is a fourth VL domain;
L₁ is a first linker sequence;
L₂ is a second linker sequence;
C_{H4-1} is a first antibody constant heavy chain 4 (CH4) domain;
C_{H4-2} is a second antibody CH4 domain;
C_{H4-3} is a third antibody CH4 domain;
C_{H4-4} is a fourth antibody CH4 domain;
C_{H1} is an antibody constant heavy chain 1 (CH1) domain;
hinge is an antibody hinge region;
Fc₁ is a first antibody Fc region;
Fc₂ is a second antibody Fc region; and
C_{L} is an antibody light chain constant (CL) domain;
wherein the first VH domain and the first VL domain form a first antigen binding site that specifically binds a first antigen, the second VH domain and the second VL domain form a second antigen binding site that specifically binds a second antigen, the third VH domain and the third VL domain form a third antigen binding site that specifically binds a third antigen, and the fourth VH domain and the fourth VL domain form a fourth antigen binding site that specifically binds a fourth antigen;
wherein the first and/or second antibody CH4 domains comprise at least one amino acid substitution that promotes association of the first and second antibody CH4 domains;
wherein the third and/or fourth antibody CH4 domains comprise at least one amino acid substitution that promotes association of the third and fourth antibody CH4 domains; and
wherein the first and/or second antibody Fc regions comprise at least one amino acid substitution that promotes association of the first and second antibody Fc regions.

132. The multispecific antibody of claim 131, wherein the first and second antibody CH4 domains are human IgM CH4 domains or human IgE CH4 domains.

133. The multispecific antibody of claim 132, wherein the first and/or second antibody CH4 domains each comprise at least one amino acid substitution relative to the amino acid sequence of SEQ ID NO: 1 or 4 that promotes association of the first and second antibody CH4 domains.

134-145. (canceled)

146. The multispecific antibody of claim 131, wherein the third and fourth antibody CH4 domains are human IgM CH4 domains or human IgE CH4 domains.

147. The multispecific antibody of claim 146, wherein the third and/or fourth antibody CH4 domains each comprise at least one amino acid substitution relative to the amino acid sequence of SEQ ID NO: 1 or 4 that promotes association of the third and fourth antibody CH4 domains.

148-159. (canceled)

160. The multispecific antibody of claim 131, wherein the first and second antibody Fc regions are human IgG Fc regions.

161-165. (canceled)

166. The multispecific antibody of claim 131, wherein:
(a) the first and second antigens are different;
(b) the third and fourth antigens are different;
(c) the first and third antigens are the same; and/or
(d) the second and fourth antigens are the same.

167-171. (canceled)

172. The multispecific antibody of claim 131, wherein at least one of the first, second, third, and fourth antigen binding sites specifically binds human Dectin-1.

173-176. (canceled)

177. A polynucleotide encoding the multispecific antibody of claim 1.

178. A vector comprising the polynucleotide of claim 177.

179. An isolated host cell comprising the polynucleotide of claim 177.

180-185. (canceled)

186. A method of producing a multispecific antibody, comprising culturing the host cell of claim 179 under conditions suitable for production of the multispecific antibody.

187-190. (canceled)

191. A pharmaceutical composition comprising the multispecific antibody of claim 1 and a pharmaceutically acceptable carrier.

192. A method of treating a disease or disorder, comprising administering an effective amount of the composition of claim 191 to an individual in need thereof.

193-201. (canceled)


SEQUENCE LISTING (108 sequences)

SEQ ID NO: 1 - protein, 109 aa, synthetic construct
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKS

SEQ ID NO: 2 - protein, 109 aa, synthetic construct
VALHRPDVYLLPPAREQLNLRESATISCAVTGFSPADVFVQWMQRGQPLSPEKYVTSAPMPEPQAPGRYFAVSILTVSEEEWNTGETYTCVVAHEALPNRVTERTVDKS

SEQ ID NO: 3 - protein, 109 aa, synthetic construct
VALHRPDVYLLPPAREQLNLRESATIWCLVTGFSPADVFVQWMQRGQPLSPEKYVTSAPMPEPQAPGRYFAHSILTVSEEEWNTGETYTCVVAHEALPNRVTERTVDKS

SEQ ID NO: 4 - protein, 100 aa, synthetic construct
PEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVS

SEQ ID NO: 5 - protein, 100 aa, synthetic construct
PEVYAFATPEWPGSRDKRTLSCAIQNFMPEDISVQWLHNEVQLPDARHSTTQPRKTKGSGFFVVSRLEVTRAEWEQKDEFICRAVHEAASPSQTVQRAVS

SEQ ID NO: 6 - protein, 100 aa, synthetic construct
PEVYAFATPEWPGSRDKRTLWCLIQNFMPEDISVQWLHNEVQLPDARHSTTQPRKTKGSGFFVFSRLEVTRAEWEQKDEFICRAVHEAASPSQTVQRAVS

SEQ ID NO: 7 - protein, 103 aa, synthetic construct
PEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTID

SEQ ID NO: 8 - protein, 103 aa, synthetic construct
PEVHLLPPPSEELALNELVTLSCAARGFSPKDVLVRWLQGSQELPREKYLTWASRQEPSQGTTTFAVVSILRVAAEDWKKGDTFSCMVGHEALPLAFTQKTID

SEQ ID NO: 9 - protein, 103 aa, synthetic construct
PEVHLLPPPSEELALNELVTLWCLARGFSPKDVLVRWLQGSQELPREKYLTWASRQEPSQGTTTFAVTSILRVAAEDWKKGDTFSCMVGHEALPLAFTQKTID

SEQ ID NO: 10 - protein, 97 aa, synthetic construct
PQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLS

SEQ ID NO: 11 - protein, 97 aa, synthetic construct
PQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLS

SEQ ID NO: 12 - protein, 97 aa, synthetic construct
PQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLS

SEQ ID NO: 13 - protein, 247 aa, Homo sapiens
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 14 - protein, 201 aa, Homo sapiens
MEYHPDLENLDEDGYTQLHFDSQSNTRIAVVSEKGSCAASPPWRLIAVILGILCLVILVIAVVLGTMGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 15 - protein, 8 aa, synthetic construct
GYTFTDYY

SEQ ID NO: 16 - protein, 8 aa, synthetic construct
INPNSGDT

SEQ ID NO: 17 - protein, 11 aa, synthetic construct
ARNSGSYSFGY

SEQ ID NO: 18 - protein, 6 aa, synthetic construct
QGISSW

SEQ ID NO: 19 - skipped

SEQ ID NO: 20 - protein, 9 aa, synthetic construct
QQAYSFPFT

SEQ ID NO: 21 - protein, 4 aa, synthetic construct
DYYI

SEQ ID NO: 22 - protein, 17 aa, synthetic construct
WINPNSGDTNYAQKFQG

SEQ ID NO: 23 - protein, 9 aa, synthetic construct
NSGSYSFGY

SEQ ID NO: 24 - protein, 11 aa, synthetic construct
RASQGISSWLA

SEQ ID NO: 25 - protein, 7 aa, synthetic construct
GASSLQS

SEQ ID NO: 26 - protein, 9 aa, synthetic construct
QQAYSFPFT

SEQ ID NO: 27 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSS

SEQ ID NO: 28 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIE

SEQ ID NO: 29 - protein, 121 aa, synthetic construct [sequence omitted]

SEQ ID NO: 30 - protein, 106 aa, synthetic construct [sequence omitted]

SEQ ID NO: 31 - protein, 459 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGVALHRPDVYLLPPAREQLNLRESATISCAVTGFSPADVFVQWMQRGQPLSPEKYVTSAPMPEPQAPGRYFAVSILTVSEEEWNTGETYTCVVAHEALPNRVTERTVDKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 222 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPFTFGPGTKVDIERTVALHRPDVYLLPPAREQLNLRESATIWCLVTGFSPADVFVQWMQRGQPLSPEKYVTSAPMPEPQAPGRYFAHSILTVSEEEWNTGETYTCVVAHEALPNRVTERTVDKSTGEC

SEQ ID NO: 33 - protein, 460 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGYWGQGTLVTVSSASTKGVALHRPDVYLLPPAREQLNLRESATISCAVTGFSPADVFVQWMQRGQPLSPEKYVTSAPMPEPQAPGRYFAVSILTVSEEEWNTGETYTCVVAHEALPNRVTERTVDKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 450 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 35 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 36 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 686 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGVALHRPDVYLLPPAREQLNLRESATISCAVTGFSPADVFVQWMQRGQPLSPEKYVTSAPMPEPQAPGRYFAVSILTVSEEEWNTGETYTCVVAHEALPNRVTERTVDKGGGGSGGGGSQVQLVQSGAEVKKPGASVKVSCKASGYTFTDYYMHWVRQAPGQGLEWMGWINPNSGDTNYAQKFQGRVTMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 687 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGVALHRPDVYLLPPAREQLNLRESATISCAVTGFSPADVFVQWMQRGQPLSPEKYVTSAPMPEPQAPGRYFAVSILTVSEEEWNTGETYTCVVAHEALPNRVTERTVDKGGGGSGGGGSQVQLVQSGAEVKKPGASVKVSCKASGYTFTDYYMHWVRQAPGQGLEWMGWINPNSGDTNYAQKFQGRVTMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 223 aa, synthetic construct
DIVLTQSPGTLSLSPGERATLSCRASQSVSSSYLAWYQQKPGQAPRLLISGASSRATGIPDRFSGSGSGTDFTLTISRLEPEDFAVYYCQQYGSSHRTFGQGTKVEIKRTVALHRPDVYLLPPAREQLNLRESATIWCLVTGFSPADVFVQWMQRGQPLSPEKYVTSAPMPEPQAPGRYFAHSILTVSEEEWNTGETYTCVVAHEALPNRVTERTVDKSTGES

SEQ ID NO: 40 - protein, 447 aa, synthetic construct
QVQLVQSGAEVKKPGASVKVSCKASGYTFTDYYMHWVRQAPGQGLEWMGWINPNSGDTNYAQKFQGRVTMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 41 - protein, 448 aa, synthetic construct
QVQLVQSGAEVKKPGASVKVSCKASGYTFTDYYMHWVRQAPGQGLEWMGWINPNSGDTNYAQKFQGRVTMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 42 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAYGFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 688 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGVALHRPDVYLLPPAREQLNLRESATISCAVTGFSPADVFVQWMQRGQPLSPEKYVTSAPMPEPQAPGRYFAVSILTVSEEEWNTGETYTCVVAHEALPNRVTERTVDKSCGGGGSGGGGSQVQLVQSGAEVKKPGASVKVSCKASGYTFTDYYMHWVRQAPGQGLEWMGWINPNSGDTNYAQKFQGRVTMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 689 aa, synthetic construct
QVQLQESGPGLVKPSGTLSLTCAVSGGSISSSNWWSWVRQPPGKGLEWIGEIYHDGSTDYFPSLKSRVTISVDKSKNQFSLKLSSVTAADTAVYYCARDNWGFDYWGQGTLVTVSSASTKGVALHRPDVYLLPPAREQLNLRESATISCAVTGFSPADVFVQWMQRGQPLSPEKYVTSAPMPEPQAPGRYFAVSILTVSEEEWNTGETYTCVVAHEALPNRVTERTVDKSCGGGGSGGGGSQVQLVQSGAEVKKPGASVKVSCKASGYTFTDYYMHWVRQAPGQGLEWMGWINPNSGDTNYAQKFQGRVTMTRDTSISTAYMELSRLRSDDTAVYYCARNTGAYSFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 223 aa, synthetic construct
DIVLTQSPGTLSLSPGERATLSCRASQSVSSSYLAWYQQKPGQAPRLLISGASSRATGIPDRFSGSGSGTDFTLTISRLEPEDFAVYYCQQYGSSHRTFGQGTKVEIKRTVALHRPDVYLLPPAREQLNLRESATIWCLVTGFSPADVFVQWMQRGQPLSPEKYVTSAPMPEPQAPGRYFAHSILTVSEEEWNTGETYTCVVAHEALPNRVTERTVDKSTGEC

SEQ ID NO: 46 - protein, 7 aa, synthetic construct
GYTFTDY

SEQ ID NO: 47 - protein, 6 aa, synthetic construct
NPNSGD

SEQ ID NO: 48 - protein, 4 aa, synthetic construct
AYYI

SEQ ID NO: 49 - protein, 8 aa, synthetic construct
GYTFTAYY

SEQ ID NO: 50 - protein, 7 aa, synthetic construct
GYTFTAY

SEQ ID NO: 51 - protein, 17 aa, synthetic construct
WINPNSGATNYAQKFQG

SEQ ID NO: 52 - protein, 8 aa, synthetic construct
INPNSGAT

SEQ ID NO: 53 - protein, 6 aa, synthetic construct
NPNSGA

SEQ ID NO: 54 - protein, 9 aa, synthetic construct
ASGSYSFGY

SEQ ID NO: 55 - protein, 11 aa, synthetic construct
ARASGSYSFGY

SEQ ID NO: 56 - protein, 9 aa, synthetic construct
NSGSASFGY

SEQ ID NO: 57 - protein, 11 aa, synthetic construct
ARNSGSASFGY

SEQ ID NO: 58 - protein, 11 aa, synthetic construct
AANSGSYSFGY

SEQ ID NO: 59 - protein, 9 aa, synthetic construct
NAGSYSFGY

SEQ ID NO: 60 - protein, 11 aa, synthetic construct
ARNAGSYSFGY

SEQ ID NO: 61 - protein, 9 aa, synthetic construct
NSASYSFGY

SEQ ID NO: 62 - protein, 11 aa, synthetic construct
ARNSASYSFGY

SEQ ID NO: 63 - protein, 9 aa, synthetic construct
NSGAYSFGY

SEQ ID NO: 64 - protein, 11 aa, synthetic construct
ARNSGAYSFGY

SEQ ID NO: 65 - protein, 11 aa, synthetic construct
ARNSGSYAFGY

SEQ ID NO: 66 - protein, 9 aa, synthetic construct
NSGSYSAGY

SEQ ID NO: 67 - protein, 11 aa, synthetic construct
ARNSGSYSAGY

SEQ ID NO: 68 - protein, 9 aa, synthetic construct
NSGSYSFAY

SEQ ID NO: 69 - protein, 11 aa, synthetic construct
ARNSGSYSFAY

SEQ ID NO: 70 - protein, 9 aa, synthetic construct
NSGSYSFGA

SEQ ID NO: 71 - protein, 11 aa, synthetic construct
ARNSGSYSFGA

SEQ ID NO: 72 - protein, 9 aa, synthetic construct
QQAASFPFT

SEQ ID NO: 73 - protein, 9 aa, synthetic construct
QQAFSFPFT

SEQ ID NO: 74 - protein, 9 aa, synthetic construct
AQAYSFPFT

SEQ ID NO: 75 - protein, 9 aa, synthetic construct
QAAYSFPFT

SEQ ID NO: 76 - protein, 9 aa, synthetic construct
QQAYAFPFT

SEQ ID NO: 77 - protein, 9 aa, synthetic construct
QQAYSAPFT

SEQ ID NO: 78 - protein, 9 aa, synthetic construct
QQAYSFAFT

SEQ ID NO: 79 - protein, 9 aa, synthetic construct
NSGSYAFGY

SEQ ID NO: 80 - protein, 9 aa, synthetic construct
QQAYSFPAT

SEQ ID NO: 81 - protein, 9 aa, synthetic construct
QQAYSFPFA

SEQ ID NO: 82 - protein, 118 aa, synthetic construct
QVQLVQSGAEVKKPGASVKVSCKSSGYTFTAYYIHWVRQAPGQGLEWMGWINPNSGDTNYAQKFQGRITMTRDTSISTAYLELSRLRSDDTAVFYCARNSGSYSFGYWGQGTLVTVSS

SEQ ID NO: 83 - protein, 118 aa, synthetic construct
QVQLVQSGAEVKKPGASVKVSCKSSGYTFTDYYIHWVRQAPGQGLEWMGWINPNSGATNYAQKFQGRITMTRDTSISTAYLELSRLRSDDTAVFYCARNSGSYSFGYWGQGTLVTVSS

SEQ ID NO: 84 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARASGSYSFGYWGQGTLVTVSS

SEQ ID NO: 85 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSASFGYWGQGTLVTVSS

SEQ ID NO: 86 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCAANSGSYSFGYWGQGTLVTVSS

SEQ ID NO: 87 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNAGSYSFGYWGQGTLVTVSS

SEQ ID NO: 88 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSASYSFGYWGQGTLVTVSS

SEQ ID NO: 89 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGAYSFGYWGQGTLVTVSS

SEQ ID NO: 90 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYAFGYWGQGTLVTVSS

SEQ ID NO: 91 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSAGYWGQGTLVTVSS

SEQ ID NO: 92 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFAYWGQGTLVTVSS

SEQ ID NO: 93 - protein, 118 aa, synthetic construct
QVQLVQSGAEVKKPGASVKVSCKSSGYTFTDYYIHWVRQAPGQGLEWMGWINPNSGDTNYAQKFQGRITMTRDTSISTAYLELSRLRSDDTAVFYCARNSGSYSFGAWGQGTLVTVSS

SEQ ID NO: 94 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAASFPFTFGPGTKVDIE

SEQ ID NO: 95 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAFSFPFTFGPGTKVDIE

SEQ ID NO: 96 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCAQAYSFPFTFGPGTKVDIE

SEQ ID NO: 97 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQAAYSFPFTFGPGTKVDIE

SEQ ID NO: 98 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYAFPFTFGPGTKVDIE

SEQ ID NO: 99 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSAPFTFGPGTKVDIE

SEQ ID NO: 100 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFAFTFGPGTKVDIE

SEQ ID NO: 101 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPATFGPGTKVDIE

SEQ ID NO: 102 - protein, 107 aa, synthetic construct
DIQMTQSPSSVSASVGDRVTITCRASQGISSWLAWYQQKPGKAPKLLIFGASSLQSGVPSRFSGSGSGTDFTLTVSSLQPEDFATYYCQQAYSFPFAFGPGTKVDIE

SEQ ID NO: 103 - protein, 14 aa, synthetic construct
EPKRSDKTHTCPPC

SEQ ID NO: 104 - protein, 5 aa, synthetic construct
GGGGS

SEQ ID NO: 105 - protein, 12 aa, synthetic construct
GGGSGGGSGGGS

SEQ ID NO: 106 - protein, 15 aa, synthetic construct
GGGGSGGGGSGGGGS

SEQ ID NO: 107 - protein, 20 aa, synthetic construct
GGGGSGGGGSGGGGSGGGGS

SEQ ID NO: 108 - protein, 9 aa, synthetic construct
SATHTCPPC